(19) United States
(12) Patent Application Publication
(10) Pub. No.: US 2018/0250308 A1
(43) Pub. Date: Sep. 6, 2018

(54) NR2B SELECTIVE NMDA-RECEPTOR ANTAGONISTS FOR TREATMENT OF IMMUNE-MEDIATED INFLAMMATORY DISEASES

(71) Applicant: WESTFALISCHE WILHELMS-UNIVERSITAT MUNSTER, Munster (DE)

(72) Inventors: Stefan Bittner, Munster (DE); Sven Meuth, Munster (DE); Bernhard Wunsch, Munster (DE); Dirk Schepmann, Munster (DE)

(21) Appl. No.: 15/755,390

(22) PCT Filed: Aug. 24, 2016
(86) PCT No.: PCT/EP2016/069975

§ 371 Date: Feb. 26, 2018

(30) Foreign Application Priority Data
Aug. 31, 2015 (LU) 92809

(51) Int. Cl. A61K 31/55; A61P 37/02; A61K 45/06

(52) U.S. Cl. CPC A61K 31/55; A61P 37/02; A61K 31/57; A61K 2300/00; A61K 45/06

(57) ABSTRACT

The present invention provides novel means and methods for treatment auf immunemediated inflammatory diseases.

### BACKGROUND

Immune-mediated inflammatory diseases (IMIDs) are a group of chronic and highly disabling diseases that share common inflammatory pathways. The term includes a wide variety of illnesses, such as Crohn's disease (CD), ulcerative colitis (UC), psoriasis, rheumatoid arthritis (RA), multiple sclerosis (MS) and systemic lupus erythematosus (SLE). The immune dysregulation accompanying IMIDs may affect any organ system and result in significant morbidity, reduced quality of life (QoL) and premature death. IMIDs are fairly common, with an incidence of 5% to 7% of the population in Western countries. Frequently, multiple IMIDs co-exist within the same patient, and various IMIDs may co-exist within the same family. Genetic factors as well as environmental factors such as infection and trauma are thought to be crucial determinants of susceptibility. IMID aetiology is still the topic of ongoing research, however, investigators stipulated that cytokine dysregulation is pivotal to the pathophysiology of IMIDs. Commonly, IMIDs are associated with a relative over-expression of cytokines, such as TNF-α in RA, yet a relative under-expression of cytokines may be equally important in disease pathogenesis, for example interleukin 10 (IL10) deficiency. As an exemplary IMID, multiple sclerosis (MS) is a chronic, autoimmune inflammatory disease of the central nervous system (CNS) characterized by axonal degeneration and demyelination (Calabresi P A., Am Fam Physician. 2004 Nov. 15;70(10):1935). The cause of MS is still unclear but it appears to be due to multifactorial aspects such as genetic predispositions and non-genetic triggers such as viral infections, commensal gut bacteria or environmental factors. (Goldenberg M M, P T. 2012 March;37(3):175-84).

Treatment of IMIDs focuses on the rapid control of inflammation, prevention of tissue damage, improvement of quality of life (QoL) and, if possible, long-term disease remission. Despite the availability of a considerable range of therapeutic agents including corticosteroids, immunosuppressants, and “biologicals” targeting key components of the dysregulated immune response underlying the disease, these goals are rarely met in patients, presumably due to failure to address and effectively treat the underlying immunopathology. Moreover, treatment with common IMID therapeutics often results in severe side-effects and/or may increase the risk of development of secondary diseases. Since there are no curative therapeutics available and safety of administered drugs is an important consideration, new therapeutic targets and approaches are urgently needed (Köhr G Cell Tissue Res. 2006;326(2):439-46, Kuek A et al. Postgrad Med J. 2007 April;83(978):251-60.)

N-methyl-D-aspartate receptors (NMDARs) are glutamate-gated ion channels reported to be widely expressed in neurons of the central nervous system. NMDARs occur as multiple subtypes which differ in their subunit composition and in their biophysical and pharmacological properties. To date, seven NMDAR subunits have been identified: one NR1, four NR2 (A-D), and two NR3 (A, B) subunits. Most native NMDARs occur as heterotetrameric assemblies composed of two glycine-binding NR1 and two glutamate-binding NR2 subunits (e.g., NR1/NR2A, NR1/NR2B, and/or NR1/NR2A/NR2B). Investigators have linked the subunit composition and compartmental localization of NMDARs in neurons to channel activity and downstream signaling. In the prior art, NMDA receptors have been reported to play a pivotal role in the regulation of neuronal communication and synaptic function in the central nervous system. Traditionally, NMDARs are best known for their role in excitotoxicity, a process during which excessive glutamate release causes overactivation of NMDARs, accumulation of intracellular calcium and, eventually, neuronal death. NMDARs have hence triggered an intense interest as potential therapeutic drug targets for developing treatments for neurological diseases. Several NMDAR antagonists were developed, including uncompetitive NMDAR antagonists binding to the NMDA ion channel and competitive antagonists acting at the agonist-binding domain on the NR2 subunits. Whereas said “first generation” NMDAR antagonist exhibit a lack of subunit selectivity and thus usually discriminate poorly between NMDAR subtypes, other NMDAR antagonists including ifenprodil and derivatives thereof tend to act more selective towards NMDAR subunits, albeit still being limited in terms of affinity and/or specificity, and, in consequence, drug safety. Further, said antagonists may exhibit unfavourable pharmacokinetics. Other candidate sites for therapeutic ligand binding, including the NR1 NTD, the ABD dimer interface and the linker region connecting the ABDs to the transmembrane segments have been proposed. Improved NMDAR antagonists specifically binding to the NR2B subunit of the receptor with high affinity have recently been provided in WO2010122134 A1. However, according to the prevailing opinion in the prior art linking NMDAR expression predominantly to neuronal cells in the CNS, the action of NMDAR antagonists has so far been almost exclusively evaluated with regards to neurological and neurodegenerative diseases (Gogas K R Curr Opin Pharmacol. 2006;6(1):68-74.).

In view of the above, there is still an urgent need in the art for providing novel and improved therapeutic approaches for treating immune-mediated inflammatory diseases, such as MS, preferably exhibiting fewer adverse side effects and an improved clinical efficacy. It is the object of the present invention to comply with this need.

### SUMMARY

The present invention provides novel means and methods for treatment of immune-mediated inflammatory diseases.

More specifically, the invention relates to compounds of the general formula (I) as given as follows and/or racemates, enantiomers, diastereomers, solvates, solvates, hydrates and pharmaceutically accepted salts and/or esters thereof for use in a method of treatment of immune-mediated-inflammatory diseases:

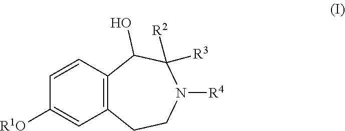

wherein:
R¹ is selected from the group comprising hydrogen; linear or branched C₁-C₈-alkyl; C₂-C₈-alkenyl; C₃-C₈-cycloalkyl; C₆-C₁₀-aryl; C₄-C₁₀-cycloalkylalkyl wherein the cycloalkyl group has 3 to 6 carbon atoms and the alkyl group has 1 to 4 carbon atoms; and/or C₇-C₁₄-arylalkyl wherein the aryl group has 6 to 10 carbon atoms and the alkyl group has 1 to 4 carbon atoms;
R² is selected from the group comprising hydrogen; linear or branched C₁-C₈-alkyl; C₂-C₈-alkenyl; C₃-C₈-cycloalkyl; C₆-C₁₀-aryl; C₄-C₁₀-cycloalkylalkyl wherein the cycloalkyl group has 3 to 6 carbon atoms and the alkyl group has 1 to 4 carbon atoms; C₇-C₁₄-arylalkyl wherein the aryl group has 6 to 10 carbon atoms and the alkyl group has 1 to 4 carbon atoms;
R³ is selected from the group comprising hydrogen; linear or branched C₁-C₈-alkyl; C₂-C₈-alkenyl; C₃-C₈-cycloalkyl; C₆-C₁₀-aryl; C₄-C₁₀-cycloalkylalkyl wherein the cycloalkyl group has 3 to 6 carbon atoms and the alkyl group has 1 to 4 carbon atoms; C₇-C₁₄-arylalkyl wherein the aryl group has 6 to 10 carbon atoms and the alkyl group has 1 to 4 carbon atoms; linear or branched alkyl groups of the type —CₙH₂ₙ-U-D wherein n is 1, 2, 3 or 4, U is selected from the group comprising O, CO, COO, CONH, S, guanidine and/or NH and D is selected from the group comprising H and/or C₁-C₃-alkyl; —CH₂-C₆H₄—X wherein X is selected from the group comprising OH, SH, C₁-C₃-alkyl and/or NH₂; —CH₂-imidazole; —CH₂-indole; —CH₂-(furanyl-3-yl); —CH₂— (pyridyl-3-yl) and/or —CH₂-(imidazolyl-3-yl); or
R² and R³ together with the carbon atom to which they are attached form a 5- to 7-membered non aromatic carbocycle or heterocycle comprising from 1 to 3 hetero atoms selected from the group comprising O, N and/or S;
R⁴ is selected from the group comprising hydrogen; C₁-C₁₀-alkyl; —W and/or —Y—Z;
Y is selected from the group comprising C₁-C₆-alkyl; C₂-C₆-alkenyl; C₂-C₆-alkynyl, C₃-C₆-cycloalkyl; C₄-C₁₀-cycloalkylalkyl wherein the cycloalkyl group has 3 to 6 carbon atoms and the alkyl group has 1 to 4 carbon atoms; C₆-C₁₀-aryl; C₇-C₁₄-arylalkyl wherein the aryl group has 6 to 10 carbon atoms and the alkyl group has 1 to 4 carbon atoms;
   C₁-C₆-alkyl comprising at least one moiety independently selected from the group comprising oxygen, sulphur, SO, SO₂, CO, NH, N(C₁-C₃-alkyl) and/or T;
   a 3- to 6-membered aromatic or non aromatic carbocycle or heterocycle containing at least one of O, N or S as heteroatoms; and/or a structural element comprising a 3- to 6-membered aromatic or non aromatic carbocycle or heterocycle comprising from 1 to 3 heteroatoms selected from the group comprising O, N and/or S and a group selected from the group comprising oxygen, sulphur, SO, SO₂, CO, NH, N(C₁-C₃-alkyl) and/or C₁-C₆-alkyl comprising at least one moiety independently selected from the group comprising oxygen, sulphur, SO, SO₂, CO, NH and/or N(C₁-C₃-alkyl);
T is selected from the group comprising

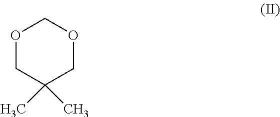

W is selected from the group comprising

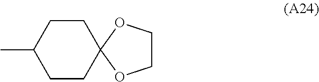

Z is selected from the group comprising mono-, bi- or tricyclic aromatic or non aromatic carbocycles or heterocycles comprising from 1 to 3 heteroatoms selected from the group comprising O, N and/or S, wherein the carbocycle or heterocycle optionally is substituted by at least one group selected from the group comprising halogen, cyano, OH, CF₃, C₁-C₄-alkyloxy and/or C₁-C₆-alkyl; or
R³ and R⁴ together with the ring atoms to which they are attached form a 5- to 7-membered non aromatic heterocycle comprising from 1 to 3 heteroatoms selected from the group comprising O, N and/or S.

In compounds according to the invention R² may be hydrogen and R³ may be a side chain of an amino acid selected from the group comprising hydrogen; linear or branched C₁-C₄-alkyl; linear or branched alkyl groups of the type —CₙH₂ₙ—U-D wherein n is 1, 2, 3 or 4, U is selected from the group comprising O, CO, COO, CONH, S, guanidine and/or NH, and D is selected from the group comprising H and/or methyl; —CH₂—C₆H₄—OH; —H₂-imidazole and/or —CH₂-indole.

The substituents R³ and R⁴ together with the ring atoms to which they are attached may form a 5-membered non aromatic heterocycle having one N atom.

The substituent R⁴ may be selected from the group comprising the structural elements as given as follows:

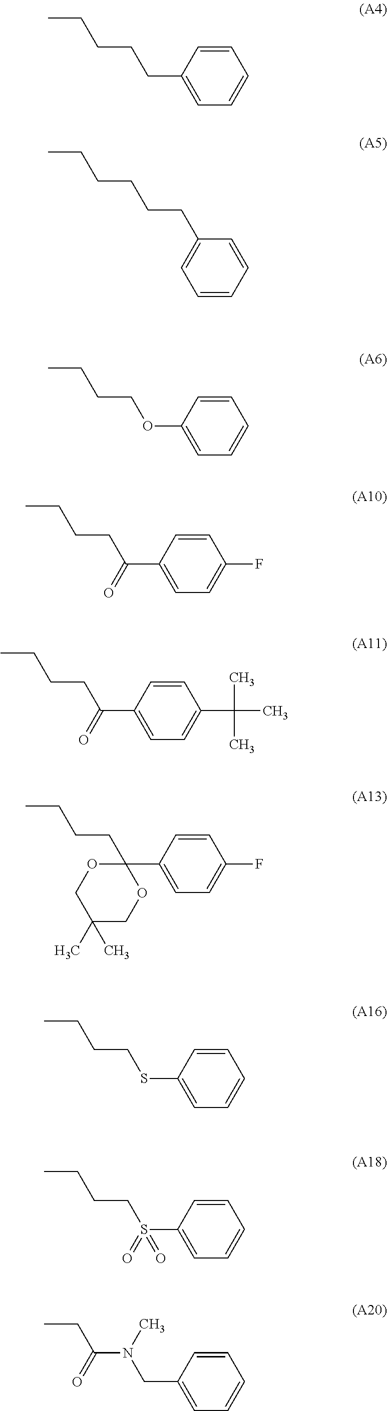

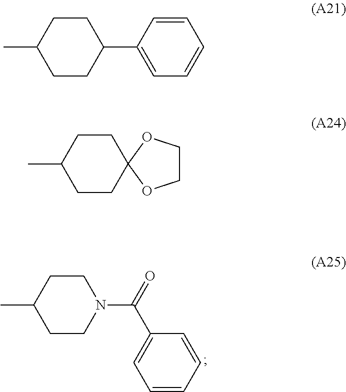

In particular, the compound for the use according to the invention may be a compound according to general formula (III) as given as follows:

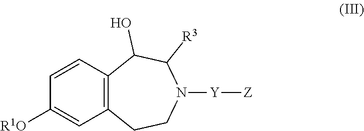

wherein:
R¹ is selected from the group comprising hydrogen; linear or branched C₁-C₆-alkyl and/or benzyl;
R³ is a side chain of an amino acid selected from the group comprising hydrogen; linear or branched C₁-C₄-alkyl; linear or branched alkyl groups of the type —CₙH₂ₙ—U-D wherein n is 1, 2, 3 or 4, U is selected from the group comprising O, CO, COO, CONH, S, guanidine and/or NH, and D is selected from the group comprising H and/or methyl; —CH₂—C₆H₄—OH; —CH₂-imidazole and/or —CH₂-indole; Y is selected from the group comprising-(CH₂)ₘ— wherein m represents 3, 4 or 5; C₃-C₅-alkenyl; C₅-C₆-cycloalkyl; C₃-C₅-alkyl comprising at least one moiety independently selected from the group comprising oxygen, sulphur, SO, SO₂, CO, NH and/or N(C₁C₃-alkyl);
   a 5- to 6-membered non aromatic carbocycle or heterocycle comprising from 1 to 3 heteroatoms selected from the group comprising O, N and/or S;
   and/or a structural element comprising a 5- to 6-membered non aromatic carbocycle and a group selected from the group comprising oxygen, sulphur, SO, SO₂, CO, NH, N(C₁-C₃-alkyl) and/or C₁-C₃-alkyl comprising at least one moiety independently selected from the group comprising oxygen, sulphur, SO, SO₂, CO, NH and/or N(C₁-C₃-alkyl);
Z is selected from the group comprising mono-, bi- or tricyclic aromatic carbocycles or heterocycles comprising from 1 to 3 heteroatoms selected from the group comprising O, N and/or S, wherein the carbocycle or heterocycle optionally is substituted by at least one group selected from the group comprising halogen, cyano, OH, CF₃, Ci-C₄-alkyloxy and/or C₁-C₆-alkyl.

Specifically, the compound may selected from the group comprising compounds according to the formulas as given as follows:

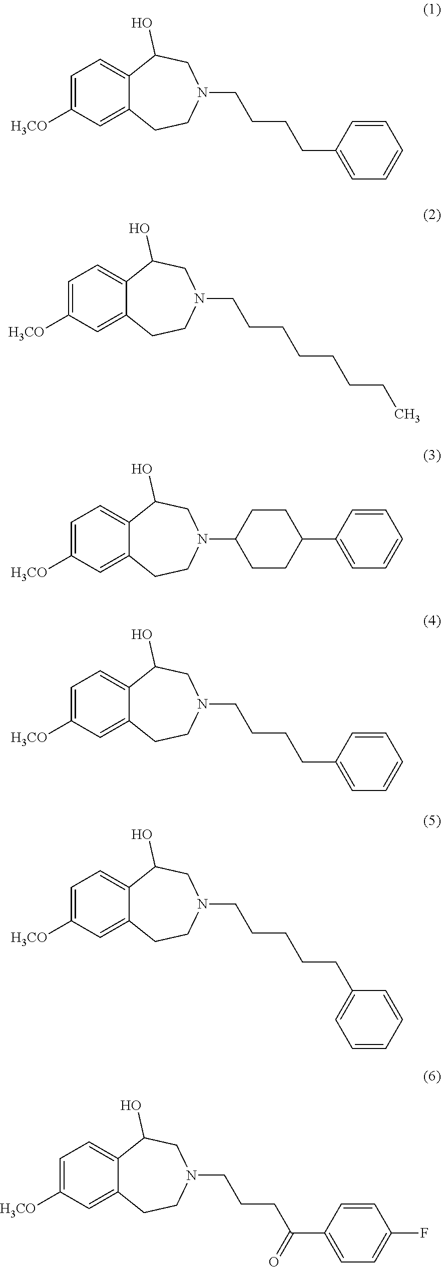 [SMILES: COc1ccc2c(c1)CCN(CCCCc1ccccc1)CC2O ; CCCCCCCCN1CCc2cc(OC)ccc2C(O)C1 ; COc1ccc2c(c1)CCN(C1CCC(c3ccccc3)CC1)CC2O ; COc1ccc2c(c1)CCN(CCCCc1ccccc1)CC2O ; COc1ccc2c(c1)CCN(CCCCCc1ccccc1)CC2O ; COc1ccc2c(c1)CCN(CCCC(=O)c1ccc(F)cc1)CC2O]

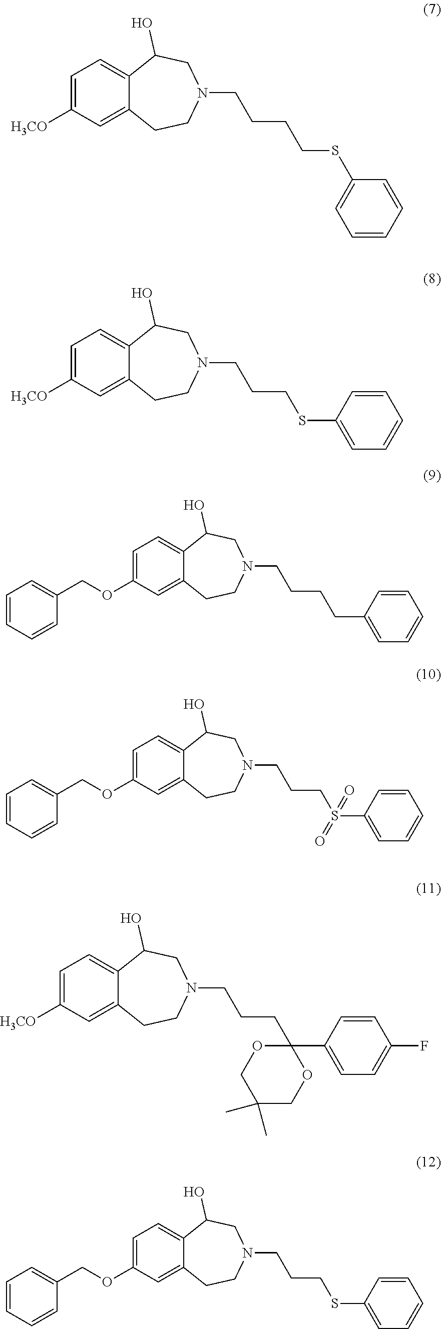 [SMILES: COc1ccc2c(c1)CCN(CCCCSc1ccccc1)CC2O ; COc1ccc2c(c1)CCN(CCCSc1ccccc1)CC2O ; OC1CN(CCCCc2ccccc2)CCc2cc(OCc3ccccc3)ccc21 ; O=S(=O)(CCCN1CCc2cc(OCc3ccccc3)ccc2C(O)C1)c1ccccc1 ; COc1ccc2c(c1)CCN(CCCC1(c3ccc(F)cc3)OCC(C)(C)CO1)CC2O ; OC1CN(CCCSc2ccccc2)CCc2cc(OCc3ccccc3)ccc21]

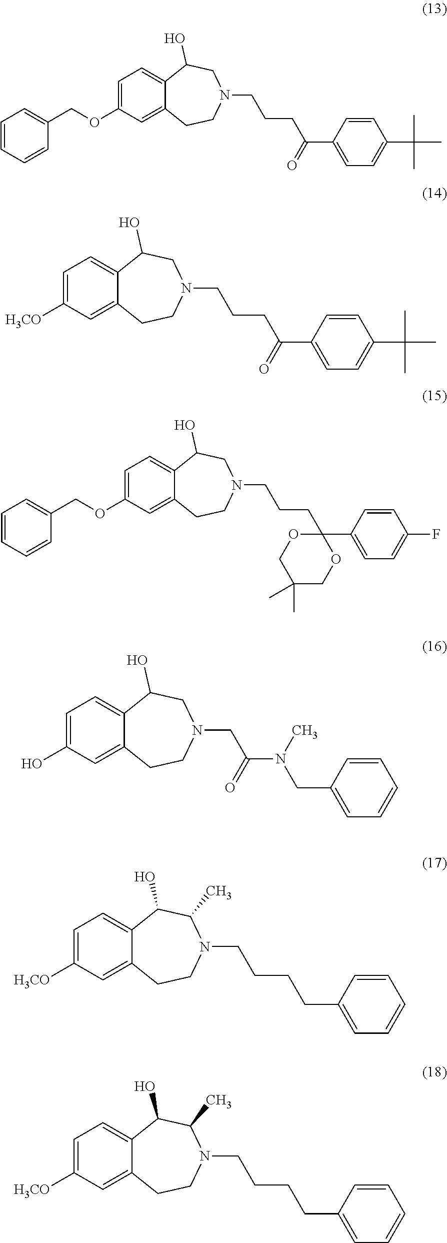 [SMILES: CC(C)(C)c1ccc(C(=O)CCCN2CCc3cc(OCc4ccccc4)ccc3C(O)C2)cc1 ; COc1ccc2c(c1)CCN(CCCC(=O)c1ccc(C(C)(C)C)cc1)CC2O ; CC1(C)COC(CCCN2CCc3cc(OCc4ccccc4)ccc3C(O)C2)(c2ccc(F)cc2)OC1 ; CN(Cc1ccccc1)C(=O)CN1CCc2cc(O)ccc2C(O)C1 ; COc1ccc2c(c1)CCN(CCCCc1ccccc1)[C@@H](C)[C@H]2O ; COc1ccc2c(c1)CCN(CCCCc1ccccc1)[C@H](C)[C@@H]2O]

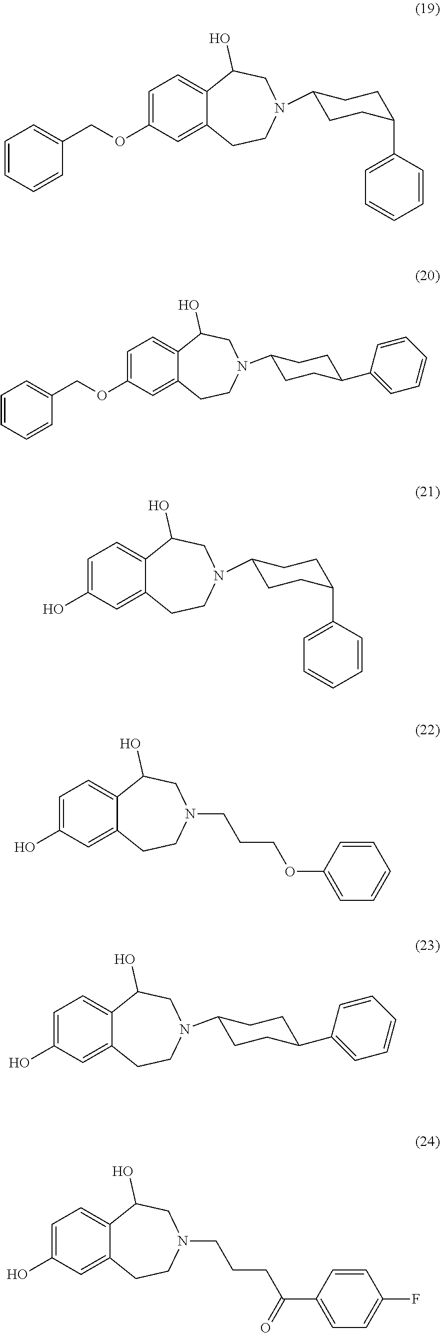 [SMILES: OC1CN(C2CCC(c3ccccc3)CC2)CCc2cc(OCc3ccccc3)ccc21 ; OC1CN(C2CCC(c3ccccc3)CC2)CCc2cc(OCc3ccccc3)ccc21 ; Oc1ccc2c(c1)CCN(C1CCC(c3ccccc3)CC1)CC2O ; Oc1ccc2c(c1)CCN(CCCOc1ccccc1)CC2O ; Oc1ccc2c(c1)CCN(C1CCC(c3ccccc3)CC1)CC2O ; O=C(CCCN1CCc2cc(O)ccc2C(O)C1)c1ccc(F)cc1]

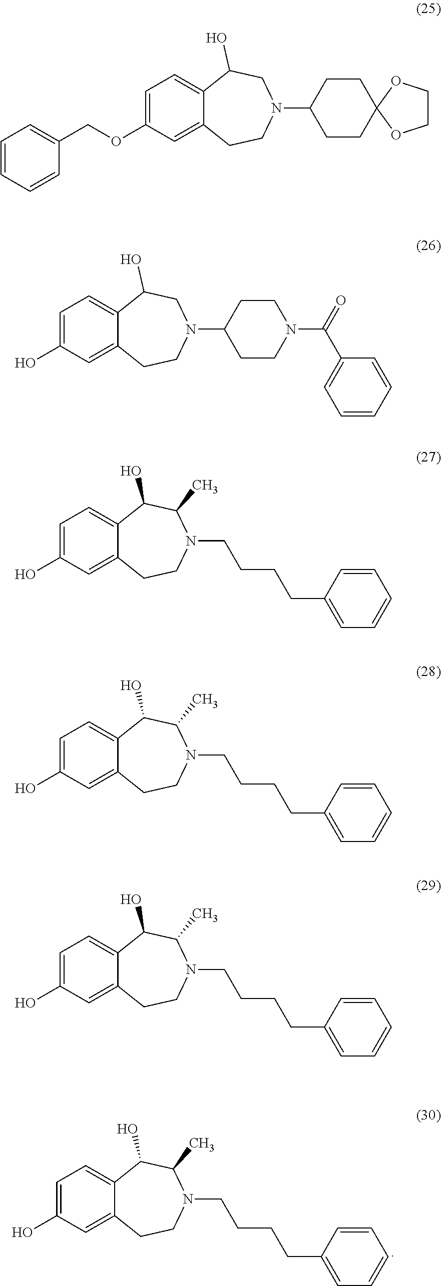 [SMILES: OC1CN(C2CCC3(CC2)OCCO3)CCc2cc(OCc3ccccc3)ccc21 ; O=C(c1ccccc1)N1CCC(N2CCc3cc(O)ccc3C(O)C2)CC1 ; C[C@@H]1[C@H](O)c2ccc(O)cc2CCN1CCCCc1ccccc1 ; C[C@H]1[C@@H](O)c2ccc(O)cc2CCN1CCCCc1ccccc1 ; C[C@H]1[C@H](O)c2ccc(O)cc2CCN1CCCCc1ccccc1 ; C[C@@H]1[C@@H](O)c2ccc(O)cc2CCN1CCCCc1ccccc1]

Compounds as described herein are envisaged for prophylactic and/or therapeutic treatment of immune-mediated inflammatory diseases which are characterized by one or more of the following features:
(i) Overexpession of proinflammatory cytokines, preferably IL-1, IL-6, IFNγ, IL-17, TNF-α; and/or overexpression of autoantibodies
(ii) Th1/Th2 cytokine disbalance; and/or Th17 disbalance and/or changes in Treg function
(iii) Autoimmune responses; and/or
(iv) Amelioration of disease symptoms by immunosuppressive therapies

The immune-infllammatory diseases treated with the compounds according to the present invention can generally be selected from a group comprising multiple sclerosis, rheumatoid arthritis, Crohn's disease, psoriasis, psoriatic arthritis, inflammatory bowel disease (IBD), ulcerative colitis (UC), systemic lupus erythematosus (SLE), Sjogren syndrome, ANCA-induced vasculitis, ankylosing spondylitis, anti-phospholipid syndrome, myasthenia gravis, Addison's disease, alopecia areata, ankylosing spondylitis, antiphospholipid antibody syndrome, antiphospholipid syndrome (primary or secondary), asthma, autoimmune gastritis, autoimmune hemolytic anemia, autoimmune hepatitis, autoimmune inner ear disease, autoimmune lymphoproliferative disease, autoimmune thrombocytopenic purpura, Balo disease, Behcet's disease, bullous pemphigoid, cardiomyopathy, celiac disease, Chagas disease, chronic inflammatory demyelinating polyneuropathy, cicatrical pemphigoid, cold agglutinin disease, degos disease, dermatitis hepatiformis, essential mixed cryoglobulinemia, Goodpasture's syndrome, Graves' disease, Guillain-Barre syndrome, Hashimoto's thyroiditis, idiopathic pulmonary fibrosis, idiopathic thrombocytopenia purpura, IgA nephropathy, juvenile arthritis, lichen planus, Meniere disease, mixed connective tissue disease, morephea, neuromyotonia, pemphigus vulgaris, pernicious anemia, polyarteritis nodosa, polychondritis, polymyalgia rheumatica, primary agammaglobulinemia, primary biliary cirrhosis, Raynaud disease (Raynaud phenomenon), Reiter's syndrome, relapsing polychondritis, rheumatic fever, Sjogren's syndrome, stiff-person syndrome (Moersch-Woltmann syndrome), Takayasu's arteritis, temporal arteritis (giant cell arteritis), uveitis, vasculitis, vitiligo, Wegener's granulomatosis and/or neuromyelitis optica, isolated CNS-vasculitis.

Treatment with the compounds as described herein preferably results in reduced activation of immune cells. The immune cells may in particular be selected from macrophages, monocytes, microglia and/or dendritic cells. Treatment is in particular 145 envisaged to result in reduced levels of

(v) Surface CD40: and/or

(vi) Surface CD86; and/or

(vii) Surface MHCII and/or

(viii) Surface CD80

in immune cells.

Treatment may, additionally or alternatively, also result in reduced release of

(i) TNFalpha; and/or

(ii) IFNgamma; and/or

(iii) IL 1β; and/or

(iv) IL6

from immune cells.

[15] It is envisaged that compounds as described herein may act as NR2B-selective NMDA receptor antagonists. In a further aspect, also provided herein are NR2B selective NMDA-receptor antagonists for use in a method of treatment of immune-mediated inflammatory diseases.

The invention further relates to a pharmaceutical composition comprising compounds as described herein as an active ingredient for use in a method of treatment of immune-mediated inflammatory diseases. The pharmaceutical composition may further comprise a pharmaceutically acceptable excipient, and/or additional agents including corticosteroids, such as prednisone and methylprednisolone, beta interferons, glatiramer acetate, dimethyl fumarate, fingolimod, teriflunomide, natalizumab, mitoxantrone, infliximab, etanercept, adalimumab, rituximab, abatacept, anakinra, alefacept, and/or efalizumab,

Further provided herein is a kit comprising a compound as described herein for use in a method of treatment of immune-mediated inflammatory diseases, and one or more additional agents selected from corticosteroids, such as prednisone and methylprednisolone, beta interferons, glatiramer acetate, dimethyl fumarate, fingolimod, teriflunomide, natalizumab, mitoxantrone, infliximab, etanercept, adalimumab, rituximab, abatacept, anakinra, alefacept, and/or efalizumab.

Use of the compounds as described herein for treatment of immune-mediated inflammatory diseases as well as methods of treatment of said diseases are also envisaged herein.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

The term “less than” or “greater than” includes the concrete number. For example, less than 20 means less than or equal to. Similarly, more than or greater than means more than or equal to, or greater than or equal to, respectively.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### DESCRIPTION OF THE FIGURES

FIG. 1: WMS14-10 treatment reduces the severity of EAE. Prophylactic (A) and therapeutic (B) treatment with 0.1 mg/kg WMS14-10 significantly ameliorates the EAE disease course. After immunization with MOG35-55 peptide, autoimmune encephalicitis (EAE) with increasing paresis similar to multiple sclerosis is induced after 10 days in wt mice and evaluated according to a clinical scoring system (0: no symptoms, 2: tail paralysis up to 6: paresis of the hind legs and forelegs). A daily application of a low dose of WMS14-10 after immunization resulted in a markedly less severe course of disease (A). Even if treatment with WMS14-10 is begun after onset of symptoms, a positive effect is achieved (treatment) (B).

FIG. 2: Reduced immune cell activation in WMS14-10 treated mice. At disease maximum dendritic cells (DCs) (A) as well as monocytes/macrophages (M+M) (B) from WMS treated mice brains show a trend of decreased activation markers in flow cytometry. Microglia from WMS treated mice expressing significantly less CD86 (C) indicate reduced microglia activation. Furthermore less DCs infiltrate the brain of treated mice (D).

FIG. 3: WMS14-10 treatment decreases secreted cytokines. Under restimulation with 1 mg/ml (MOG1) and 10 mg/ml MOG (MOG10) splenocytes from prophylactically WMS treated mice demonstrate significantly decreased secretion of INFγ(A), TNFα (B) and IL-17 (C) at disease maximum. The proliferation rate, measured as relative light units (RLU) of ATP per luciferase, of splenocytes from treated mice also decreases after restimulation with MOG (D).

FIG. 4: NR2B is upregulated upon CNS inflammation. A: No expression of NR2B (GluN2B) could be observed in histological cross sections of the spinal cord of healthy C57BL/6 mice. Results are consistent with the scientific literature since there is no function known of NR2B in the adult spinal cord. B and C: Histological cross sections of the spinal cord of mice in the maximal stage of disease. NR2B expression in EAE mouse correlates with Iba1 expression, a marker for activated microglia, in transverse lumbar spinal cord (B) as well as with primary microglia stained with CD11b (B, D). In agreement, either unstimulated or with 100 ng/ml LPS stimulated primary microglia show GluN2B mRNA expression (D). NR2B expression in transverse lumbar spinal cord under control (A) and under inflammatory (B) conditions of healthymice or mice developing spontaneous EAE.

FIG. 5: WMS14-10 treatment decreases activation markers and cytokine secretion of pMg. NR2B expression levels on stimulated and unstimulated primary murine microglia as compared to CD4+, CD8+and CD11+ cells of naive C57BL/6 mice (FIG. 5A). Upon stimulation with 100 ng/ml LPS, primary murine microglia upregulate activation markers CD40, CD86 and MHCII. Application of 2, 20 and 200 nM WMS inhibits upregulation of activation markers (FIG. 5B) and reduces secreted levels of TNFa and IL113 as assessed by ELISA (FIG. 5C).

FIG. 6: Amelioration of tissue damage due to WMS14-10 treatment. WMS14-10-mediated inhibition of NR2B and reduced activation of immune cells resulted in reduced loss of neurons on d30.

### DETAILED DESCRIPTION

According to a commonly held prejudice in the prior art, expression of NMDAR was thought to be restricted to neuronal cells throughout the CNS. Aberrant or excessive activity of the NMDAR has traditionally been associated with ischaemic brain injury/stroke, Alzheimer's disease, Parkinson's disease, Huntington's disease and chronic/neuropathic pain states. Hence, use of NMDAR antagonists blocking NMDA function has been restricted to treatment of diseases affecting the CNS which are caused by and associated with neuronal dysfunction. In WO 2010/122134 A1, novel compounds (i.e. derivatives of benzo-fused nitrogen heterocycles) capable of binding to the NR2B receptor subunit with high specificity and affinity were provided. However, following the bias in the prior art linking NMDAR activity exclusively with neuronal function and survival, intended exploitation of said compounds was restricted to treatment of diseases of the CNS, the classical field of application of compounds related to dysregulated NMDAR function.

In marked contrast to the prejudice in the prior art, the present inventors have broken new ground as regards NMDAR and the use of NMDAR subunit specific antagonists. To their surprise, the present inventors discovered that not only neurons, but also immune cells (such as microglia and monocytes) expressed the NMDAR, and in particular the NR2B receptor subunit. Moreover the inventors unexpectedly found that selective inhibitors of the NR2B subunit of the NMDAR—such as the compounds provided in WO 2010/122134 A1—can advantageously downregulate activation markers and reduce secretion of pro-inflammatory cytokines from activated immune cells, thus suggesting a major potential of known and future NR2B-selective NMDAR antagonists in treatment of immune-mediated inflammatory diseases (IMIDs). The finding was confirmed in an in vivo mouse model of multiple sclerosis 295 (EAE), showing a markedly less severe progression of disease after treatment with WMS14-14, a compound as disclosed in WO 2010/122134 A1, even if treatment was begun after symptoms had developed. Even re-stimulation of immune cells resulted in considerably attenuated re-activation after treatment with the NR2B-selective NMDA receptor, thereby implying a pivotal role of NMDA receptors in orchestrating immune responses.

Thus, in a first aspect, the present invention provides compounds according to the general formula (I)

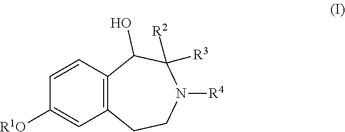

for use in a method of therapeutic and/or prophylactic treatment of immune-mediated inflammatory diseases
wherein:
R¹ is selected from the group comprising hydrogen; linear or branched C₁-C₈-alkyl; C₂-C₈-alkenyl; C₃-C₈-cycloalkyl; C₆-C₁₀-aryl; C₄-C₁₀-cycloalkylalkyl wherein the cycloalkyl group has 3 to 6 carbon atoms and the alkyl group has 1 to 4 carbon atoms; and/or C₇-C₁₄-arylalkyl wherein the aryl group has 6 to 10 carbon atoms and the alkyl group has 1 to 4 carbon atoms;
R² is selected from the group comprising hydrogen; linear or branched C₁-C₈-alkyl; C₂-C₈-alkenyl; C₃-C₈-cycloalkyl; C₆-C₁₀-aryl; C₄-C₁₀-cycloalkylalkyl wherein the cycloalkyl group has 3 to 6 carbon atoms and the alkyl group has 1 to 4 carbon atoms; C₇-C₁₄-arylalkyl wherein the aryl group has 6 to 10 carbon atoms and the alkyl group has 1 to 4 carbon atoms;
R³ is selected from the group comprising hydrogen; linear or branched C₁-C₈-alkyl; C₂-C₈-alkenyl; C₃-C₈-cycloalkyl; C₆-C₁₀-aryl; C₄-C₁₀-cycloalkylalkyl wherein the cycloalkyl group has 3 to 6 carbon atoms and the alkyl group has 1 to 4 carbon atoms; C₇-C₁₄-arylalkyl wherein the aryl group has 6 to 10 carbon atoms and the alkyl group has 1 to 4 carbon atoms; linear or branched alkyl groups of the type —CₙH₂ₙ—U-D wherein n is 1 , 2, 3 or 4, U is selected from the group comprising O, CO, COO, CONH, S, guanidine and/or NH and D is selected from the group comprising H and/or C₁-C₃-alkyl; —CH₂-C₆H₄—X wherein X is selected from the group comprising OH, SH, C₁-C₃-alkyl and/or NH₂; —CH₂-imidazole; —CH₂-indole; —CH₂-(furanyl-3-yl); —CH₂— (pyridyl-3-yl) and/or —CH₂-(imidazolyl-3-yl); or
R² and R³ together with the carbon atom to which they are attached form a 5- to 7-membered non aromatic carbocycle or heterocycle comprising from 1 to 3 hetero atoms selected from the group comprising O, N and/or S;
R⁴ is selected from the group comprising hydrogen; C₁-C₁₀-alkyl; —W and/or —Y—Z;
Y is selected from the group comprising C₁-C₆-alkyl; C₂-C₆-alkenyl; C₂-C₆-alkynyl, C₃-C₆-cycloalkyl; C₄-C₁₀-cycloalkylalkyl wherein the cycloalkyl group has 3 to 6 carbon atoms and the alkyl group has 1 to 4 carbon atoms; C₆-C₁₀-aryl; C₇-C₁₄-arylalkyl wherein the aryl group has 6 to 10 carbon atoms and the alkyl group has 1 to 4 carbon atoms;
   C₁-C₆-alkyl comprising at least one moiety independently selected from the group comprising oxygen, sulphur, SO, SO₂, CO, NH, N(C₁-C₃-alkyl) and/or T;
   a 3- to 6-membered aromatic or non aromatic carbocycle or heterocycle containing at least one of O, N or S as heteroatoms; and/or a structural element comprising a 3- to 6-membered aromatic or non aromatic carbocycle or heterocycle comprising from 1 to 3 heteroatoms selected from the group comprising O, N and/or S and a group selected from the group comprising oxygen, sulphur, SO, SO₂, CO, NH, N(C₁-C₃-alkyl) and/or C₁-C₆-alkyl comprising at least one moiety independently selected from the group comprising oxygen, sulphur, SO, SO₂, CO, NH and/or N(C₁-C₃-alkyl);
T is selected from the group comprising

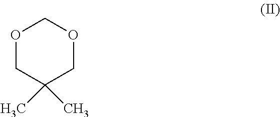

W is selected from the group comprising

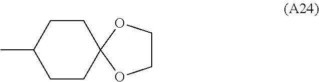

Z is selected from the group comprising mono-, bi- or tricyclic aromatic or non aromatic carbocycles or heterocycles comprising from 1 to 3 heteroatoms selected from the group comprising O, N and/or S, wherein the carbocycle or heterocycle optionally is substituted by at least one group selected from the group comprising halogen, cyano, OH, CF₃, C₁-C₄-alkyloxy and/or C₁-C₆-alkyl; or
R³ and R⁴ together with the ring atoms to which they are attached form a 5- to 7-membered non aromatic heterocycle comprising from 1 to 3 heteroatoms selected from the group comprising O, N and/or S;
   and/or racemates, enantiomers, diastereomers, solvates, hydrates, and pharmaceutically acceptable salts and/or esters thereof. The aforementioned NR2B-selective NMDAR antagonists have been shown to be therapeutically more effective in the treatment of immune-mediated inflammatory diseases such as MS than other known NMDAR antagonists. Said NR2B-selective NMDAR antagonists discussed herein are thus envisaged to be effective at lower dosages than known NMDAR antagonists in the aforementioned treatment. They might further require a lower average duration of treatment and/or result in fewer side effects.

The term “alkyl” according to the invention is to be understood as meaning straight-chain or branched alkyl groups. The term “C₁-C₈-alkyl” as used herein refers to straight-chain or branched alkyl groups having 1 to 8 carbon atoms. Preferred C₁-C₈-alkyl groups are selected from the group comprising methyl, ethyl and the isomers of propyl, butyl, pentyl, hexyl, heptyl or octyl, such as, for example, isopropyl, isobutyl, tert.-butyl, sec-butyl and/or isopentyl.

The term “alkenyl” according to the invention is to be understood as meaning straight-chain or branched alkyl groups having at least one or several double bonds. The term “C₂-C₈-alkenyl” as used herein refers to straight-chain or branched alkenyl groups having 2 to 8 carbon atoms and at least one or several double bonds. Preferred C₂-C₈-alkenyl groups are selected from the group comprising propen-1-yl, allyl, buten-1-yl, buten-2-yl and/or buten-3-yl.

The term “alkynyl” according to the invention is to be understood as meaning straight-chain or branched alkyl groups having at least one or several carbon-carbon triple bonds. The term “C₂-C₈-alkynyl” as used herein refers to straight-chain or branched alkynyl groups having 2 to 8 carbon atoms and at least one or several triple bonds. Preferred C₂-C₈-alkenyl groups are selected from the group comprising propyn-2-yl, butyn-1-yl, butyn-2-yl, pentyn-4-yl, and/or propargyl.

The term “cycloalkyl” according to the invention is to be understood as meaning carbocycles, and includes mono-, bi- and tricyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spirofused ring systems. The term “C₃-C₈-cycloalkyl” as used herein refers to carbocycles having 3 to 8 carbon atoms. Preferred cycloalkyl groups are selected from the group comprising cyclopropyl, cyclobutyl, cyclopentyl and/or cyclohexyl.

The term “C₈-C₁₀-aryl” according to the invention is to be understood as meaning aromatic groups having 6 to 10 carbon atoms. Preferably, the term “C₈-C₁₀-aryl ” refers to carbocycles. Preferred C₈-C₁₀-aryl is selected from the group comprising phenyl or naphthyl.

The terms “cycloalkylalkyl”, “cycloalkylalkenyl”, “arylalkyl”, “arylalkenyl”, and “cycloalkenylalkyl” according to the invention unless specifically stated otherwise are to be understood as meaning groups which bond by the respective last-mentioned group, for example referring to “arylalkyl” by the alkyl group.

The term “C₄-C₁₀-cycloalkylalkyl wherein the cycloalkyl group has 3 to 6 carbon atoms and the alkyl group has 1 to 4 carbon atoms” preferably is a group phenylalkyl wherein the alkyl has 1 to 4 carbon atoms, more preferably selected from the group comprising benzyl, phenylethyl and/or phenylbutyl.

The term “alkyloxy” according to the invention is to be understood as meaning an alkyl group connected to the oxy connecting atom unless specifically stated otherwise. The term “C₁-C₄₋alkyloxy” as used herein refers to an alkyloxy group having 1 to 4 carbon atoms. C₁-C₄-alkyloxy group are preferably selected from the group comprising methoxy, ethoxy, n-propoxy, isopropoxy, n-butoxy, iso-butoxy, secondary-butoxy and/or tertiary-butoxy.

As used herein, the term “heterocycle” refers to a stable 5- to 7-membered monocyclic or bicyclic heterocyclic ring or a 7- to 10-membered bicyclic heterocyclic ring which is either saturated or unsaturated, and which comprises carbon atoms and from 1 to 3 heteroatoms selected from the group comprising N, O and/or S. The term “heterocycle” includes bicyclic groups in which any of the above-defined heterocyclic rings is fused to a benzene ring. Preferred heterocycles are monocyclic heteroarylic rings.

Preferred heterocycles are selected from the group comprising furan, tetrahydrofuran, thiophene, tetrahydropyran, pyrrole, pyrrolidine, imidazole, 1,2,4-triazole, piperidine, pyridine, pyrimidine, morpholine or azacycloheptane. Further preferred heterocycles are selected from the group comprising furan-2-yl, tetrahydrofuran-2-yl, tetrahydrofuran-3-yl, tetrahydropyran-2-yl, 1,3-dioxolan-5-yl, pyrrol-1-yl, pyrrol-2-yl, pyrrolidin-1-yl, isoxazol-3-yl, isoxazol-4-yl, 1,2-di-thiazolin-5-yl, imidazol-1-yl, 1,2,4-triazol-1-yl, 1,3,4-triazol-1-yl, thiophen-2-yl, piperidin-1-yl, piperidin-4-yl, pyridin-2-yl, pyridin-3-yl, pyridin-4-yl, pyrimidin-2-yl, pyrimidin-4-yl, pyrimidin-5-yl, morpholin-1-yl, azacycloheptan-1-yl and/or benzo-1,2,3-thiadiazol-7-yl.

The term “heteroatoms” according to the invention preferably relates to N, O and S, unless specifically stated otherwise.

The term “halogen” according to the invention is to be understood as meaning fluorine, chlorine, bromine or iodine, preferably fluorine, chlorine or bromine.

The term “amino acid” according to the invention is to be understood as meaning alpha amino acids, molecules containing both amine and carboxyl functional groups attached to the same carbon, which is called the alpha-carbon. The term “amino acid” according to the invention is to be understood as being broadly defined to include any modified and unusual amino acid. Preferred amino acids are naturally occurring amino acids. Representative amino acids include, but are not limited to, the group comprising glycine, alanine, serine, threonine, arginine, lysine, aspartic acid, glutamic acid, asparagine, glutamine, phenylalanine, tyrosine, tryptophan, leucine, valine, isoleucine, cysteine, methionine, histidine and/or proline. The various alpha amino acids differ in which side chain is attached to their alpha carbon.

The term “side chains of amino acids” according to the invention is to be understood as meaning the groups attached to the alpha carbon of alpha-amino acids. For example the side chains of glycine, alanine, valine, leucine and phenylalanine are hydrogen, methyl, iso-propyl, isobutyl and benzyl, respectively.

In the compounds as described herein, R¹ is preferably selected from the group comprising hydrogen, straight-chain or branched C₁-C₆-alkyl and/or benzyl, and in particular R¹ is envisaged to be an alkyl group selected from the group comprising methyl and/or ethyl. Without wishing to be bound by theory, it is contemplated that selecting R¹ from the group comprising hydrogen, straight-chain or branched C₁-C₆-alkyl and/or benzyl can result in a substantial increase in affinity and/or selectivity of the compound towards the NR2B subunit of the NMDA receptor.

Preferably, the substituent R² of the compounds described herein is hydrogen.

It is further envisaged that in the compounds according to the invention the substituent R³ may be a side chain of an amino acid. Preferably, the substituent R³ is a side chain of a naturally occurring amino acid selected from the group comprising glycine, alanine, serine, threonine, arginine, lysine, aspartic acid, glutamic acid, asparagine, glutamine, phenylalanine, tyrosine, tryptophan, leucine, valine, isoleucine, cysteine, methionine, histidine and/or proline. Advantageously, the use of amino acids having S- or R-configuration is envisaged to provide for a synthesis of the compounds according to the invention having selected stereochemistry.

Accordingly, in the compounds according to the invention the substituent R₂ may be hydrogen and the substituent R₃ is a side chain of an amino acid selected from the group comprising hydrogen; linear or branched C₁-C₄-alkyl; linear or branched alkyl groups of the type —Cₙ-H₂ₙ—U-D wherein n is 1, 2, 3 or 4, U is selected from the group comprising O, CO, COO, CONH, S, guanidine and/or NH, and D is selected from the group comprising H and/or methyl; —CH₂—C₆H₄—OH; —CH₂-imidazole and/or —CH₂-indole.

In other preferred embodiments, the substituents R₂ and R₃ may together with the carbon atom to which they are attached form a 5-, 6- or 7-membered non-aromatic carbocycle or heterocycle, preferably a heterocycle having one N atom.

In other preferred embodiments of the compounds according to the invention the substituents R³ and R⁴ together with the ring atoms to which they are attached form a 5-membered non aromatic heterocycle having one N atom. In this embodiment the substituents R3 and R4 together with the ring atoms to which they are attached form the side chain of proline.

The substituent R⁴ can be C₆-C₉-alkyl, preferably C₇-C₈-alkyl, more preferably straight chain C₇-C₈-alkyl.

Preferably, the substituent R⁴ may be a group —Y—Z. Preferably, Y is a non aromatic group. Preferably, Y is selected from the group comprising C₃₋₅-alkyl; C₃₋₅-alkenyl; C₅-C₆-cycloalkyl; C₃₋₅-alkyl comprising at least one moiety independently selected from the group comprising oxygen, sulphur, SO, SO₂, CO, NH, N(C₁-C₃-alkyl) and/or T; a 5-to 6-membered non aromatic carbocycle or heterocycle containing at least one of O, N or S as heteroatoms; and/or a structural element comprising a 5- to 6-membered non aromatic carbocycle or heterocycle comprising from 1 to 3 heteroatoms selected from the group comprising O, N and/or S and a group selected from the group comprising oxygen, sulphur, SO, SO₂, CO, NH, N(C₁-C₃-alkyl) and/or C₁-C₃-alkyl comprising at least one moiety independently selected from the group comprising oxygen, sulphur, SO, SO₂, CO, NH and/or N(C₁-C₃-alkyl).

The length of the substituent Y preferably is the length of a C₃₋₅-alkyl group, more preferably the length of a C₄-alkyl group. Preferably, the substituent Y is —(CH₂)ₘ,— wherein m represents 3, 4 or 5, preferably 4. More preferably, the substituent Y is C₃₋₅-alkyl preferably C₄-alkyl comprising at least one moiety independently selected from the group comprising oxygen, sulphur, SO, SO₂, CO, NH, N(C₁-C₃-alkyl) and/or T.

Without wishing to be bound by theory, it is expected that the substituent Y having a length of a C₃₋₅-alkyl group, more preferably the length of a C₄-alkyl group, can advantageously result in a substantial increase in affinity and/or selectivity of the compound towards the NR2B subunit of the NMDA receptor.

The backbone of the substituent Y preferably is stiff. Preferably, the stiffness is provided by a substituent Y comprising a double bonding, or the substituent Y being a non aromatic carbocycle or heterocycle. In preferred embodiments, the substituent Y is selected from the group comprising C₃₋₅-alkenyl; C₅-C₆-cycloalkyl; a 5- to 6-membered non aromatic carbocycle or heterocycle containing at least one of O, N or S as heteroatoms; and/or a structural element comprising a 5- to 6-membered non aromatic carbocycle or heterocycle comprising from 1 to 3 heteroatoms selected from the group comprising O, N and/or S and a group selected from the group comprising oxygen, sulphur, SO, SO₂, CO, NH, N(C₁-C₃-alkyl) and/or C₁-C₃-alkyl comprising at least one moiety independently selected from the group comprising oxygen, sulphur, SO, SO2, CO, NH and/or N(C₁-C₃-alkyl).

The substituent Y preferably is a 3- to 6-membered or 5- to 6-membered aromatic or non aromatic carbocycle or heterocycle containing at least one of O, N or S as heteroatom with the provision that two adjacent heteroatoms are not simultaneously oxygen.

Preferably, the substituent Z is an aromatic group. Without wishing to be bound by theory, it is thought that the substituent Z being an aromatic group can exhibit a positive effect on the affinity and/or selectivity of the compounds to the NR2B subunit of the NMDA receptor.

The substituent Z may preferably be selected from the group comprising mono-, bi- or tricyclic aromatic carbocycles or heterocycles comprising from 1 to 3 heteroatoms selected from the group comprising O, N and/or S, wherein the carbocycle or heterocycle optionally is substituted by at least one group selected from the group comprising halogen, cyano, OH, CF₃, C₁-C₄-alkyloxy and/or C₁-C₆-alkyl.

Preferably, the substituent Z is selected from the group comprising phenyl, biphenyl, pyridine and/or pyrimidine.

Advantageously, in embodiments where the substituent R4 is a group —Y—Z, wherein the substituent Y has the length of a C₄-alkyl group and the substituent Z is an aromatic group, a group —Y—Z can result in a substantial increase in affinity and/or selectivity of the compound towards the NR2B subunit of the NMDA receptor.

In preferred embodiments, the substituent —Y—Z is selected from the group of structural elements as given as follows:

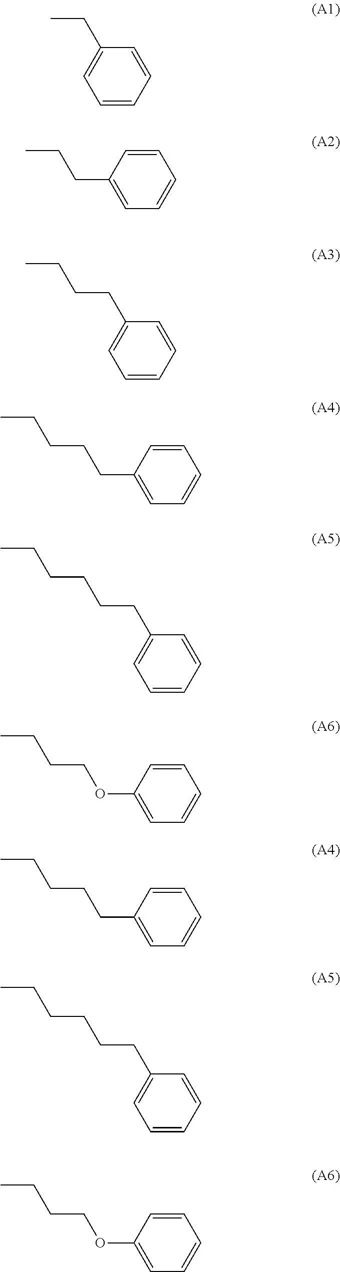

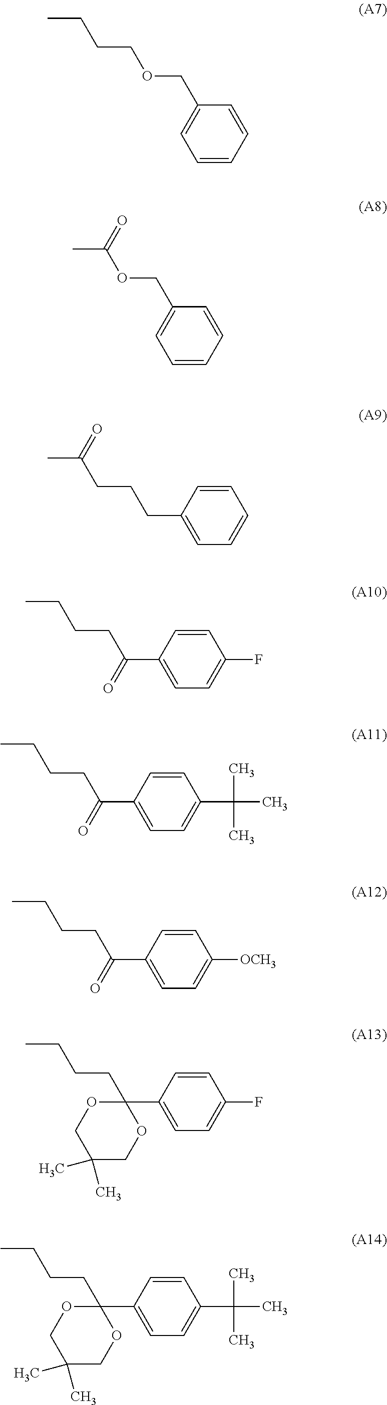

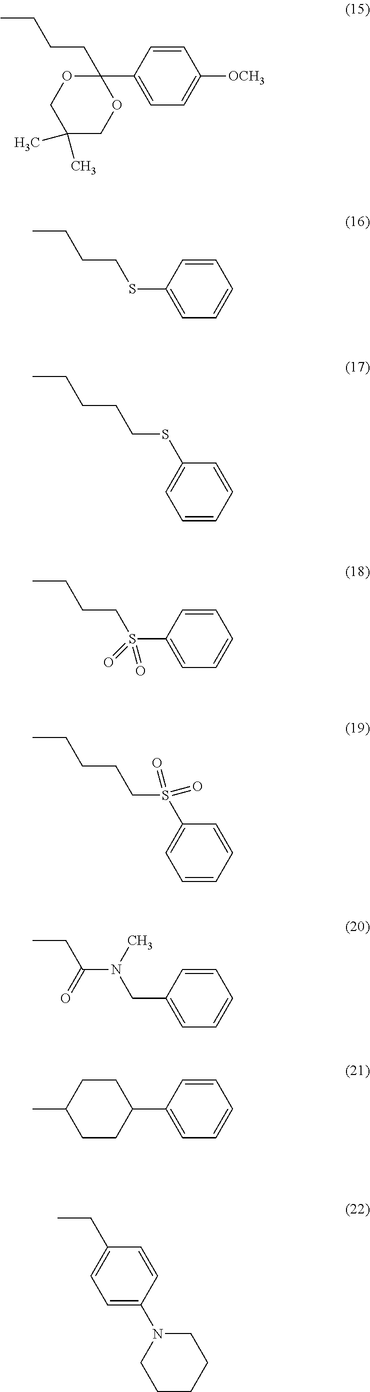

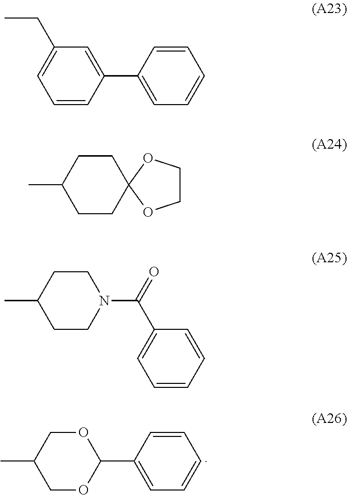

Further, the substituent R4 may preferably be selected from the group of structural elements as given as follows:

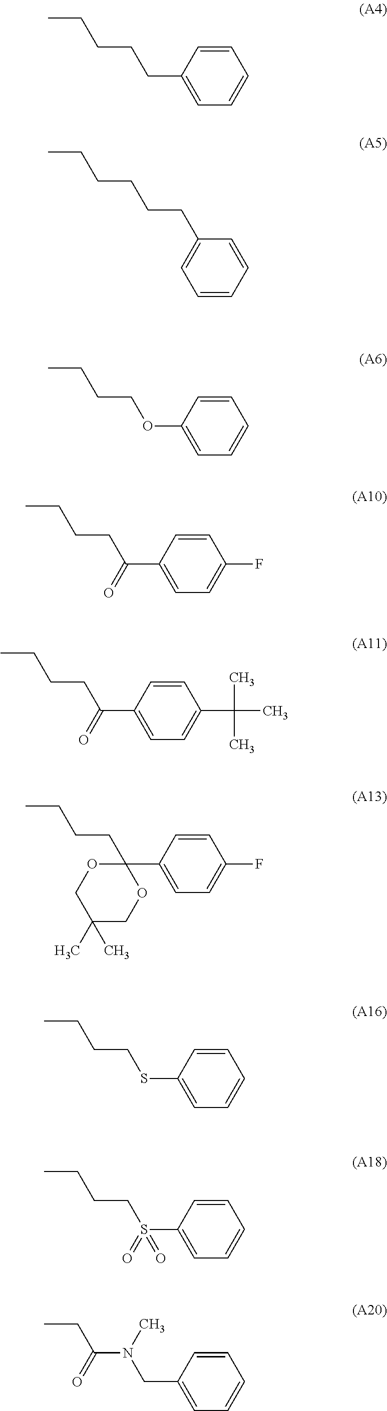

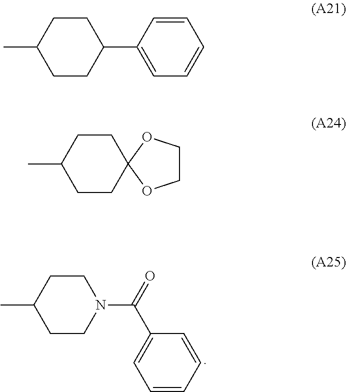

Preferably, the compound may be a compound according to the general formula (Ill) as given as follows:

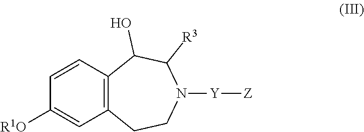

wherein:
R¹ is selected from the group comprising hydrogen; linear or branched C₁-C₆-alkyl and/or benzyl;
R³ is a side chain of an amino acid selected from the group comprising hydrogen; linear or branched C₁-C₄-alkyl; linear or branched alkyl groups of the type —CₙH₂ₙ—U-D wherein n is 1, 2, 3 or 4, U is selected from the group comprising O, CO, COO, CONH, S, guanidine and/or NH, and D is selected from the group comprising H and/or methyl; —CH₂-C₆H₄—OH; —CH₂-imidazole and/or —CH₂-indole;
Y is selected from the group comprising-(CH₂)ₘ— wherein m represents 3, 4 or 5; C₃-C₅-alkenyl; C₅-C₆-cycloalkyl; C₃-C₅-alkyl comprising at least one moiety independently selected from the group comprising oxygen, sulphur, SO, SO₂, CO, NH and/or N(C₁C₃-alkyl);
   a 5- to 6-membered non aromatic carbocycle or heterocycle comprising from 1 to 3 heteroatoms selected from the group comprising O, N and/or S;
   and/or a structural element comprising a 5- to 6-membered non aromatic carbocycle and a group selected from the group comprising oxygen, sulphur, SO, SO₂, CO, NH, N(C₁-C₃-alkyl) and/or C₁-C₃-alkyl comprising at least one moiety independently selected from the group comprising oxygen, sulphur, SO, SO₂, CO, NH and/or N(C₁-C₃-alkyl);
Z is selected from the group comprising mono-, bi- or tricyclic aromatic carbocycles or heterocycles comprising from 1 to 3 heteroatoms selected from the group comprising O, N and/or S, wherein the carbocycle or heterocycle optionally is substituted by at least one group selected from the group comprising halogen, cyano, OH, CF₃, Ci-C₄-alkyloxy and/or C₁-C₆-alkyl;

Particularly preferred for the use according to the invention are compounds being selected from the group comprising compounds according to the formulas as given as follows:

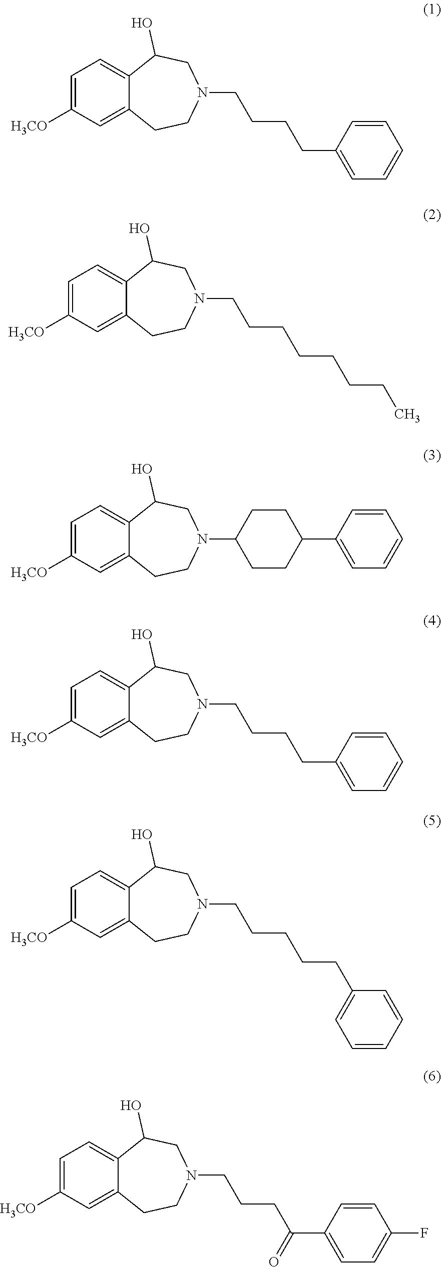 [SMILES: COc1ccc2c(c1)CCN(CCCCc1ccccc1)CC2O ; CCCCCCCCN1CCc2cc(OC)ccc2C(O)C1 ; COc1ccc2c(c1)CCN(C1CCC(c3ccccc3)CC1)CC2O ; COc1ccc2c(c1)CCN(CCCCc1ccccc1)CC2O ; COc1ccc2c(c1)CCN(CCCCCc1ccccc1)CC2O ; COc1ccc2c(c1)CCN(CCCC(=O)c1ccc(F)cc1)CC2O]

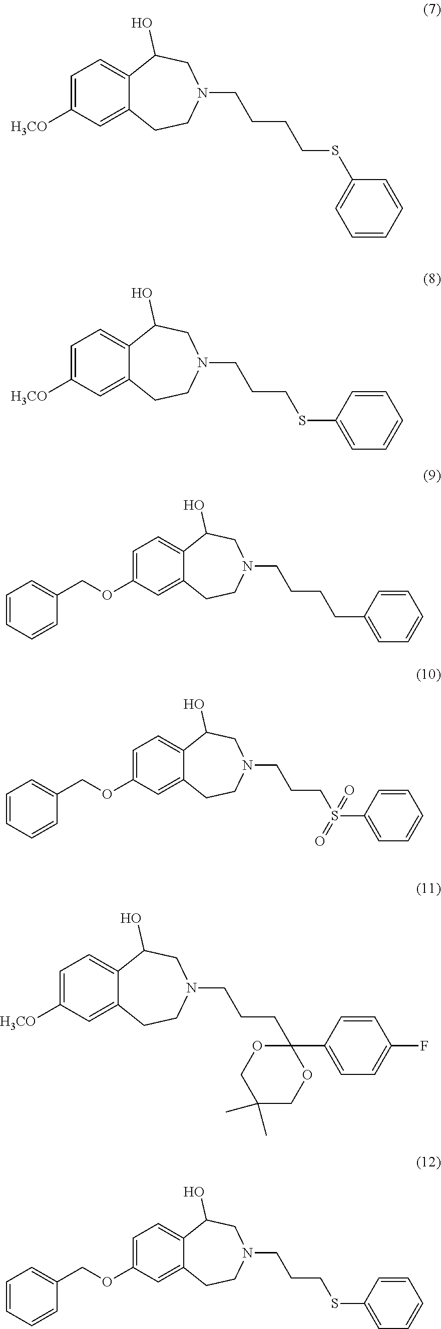 [SMILES: COc1ccc2c(c1)CCN(CCCCSc1ccccc1)CC2O ; COc1ccc2c(c1)CCN(CCCSc1ccccc1)CC2O ; OC1CN(CCCCc2ccccc2)CCc2cc(OCc3ccccc3)ccc21 ; O=S(=O)(CCCN1CCc2cc(OCc3ccccc3)ccc2C(O)C1)c1ccccc1 ; COc1ccc2c(c1)CCN(CCCC1(c3ccc(F)cc3)OCC(C)(C)CO1)CC2O ; OC1CN(CCCSc2ccccc2)CCc2cc(OCc3ccccc3)ccc21]

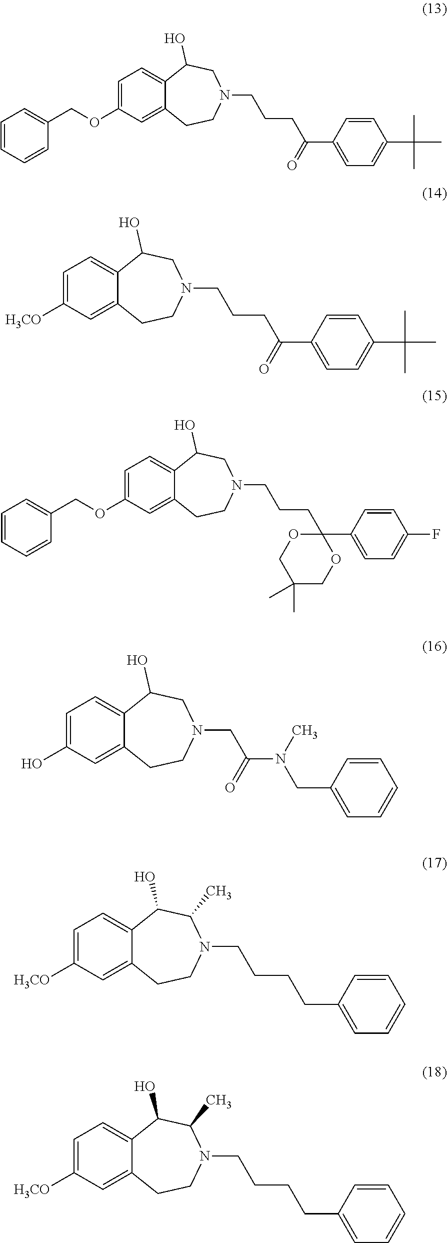 [SMILES: CC(C)(C)c1ccc(C(=O)CCCN2CCc3cc(OCc4ccccc4)ccc3C(O)C2)cc1 ; COc1ccc2c(c1)CCN(CCCC(=O)c1ccc(C(C)(C)C)cc1)CC2O ; CC1(C)COC(CCCN2CCc3cc(OCc4ccccc4)ccc3C(O)C2)(c2ccc(F)cc2)OC1 ; CN(Cc1ccccc1)C(=O)CN1CCc2cc(O)ccc2C(O)C1 ; COc1ccc2c(c1)CCN(CCCCc1ccccc1)[C@@H](C)[C@H]2O ; COc1ccc2c(c1)CCN(CCCCc1ccccc1)[C@H](C)[C@@H]2O]

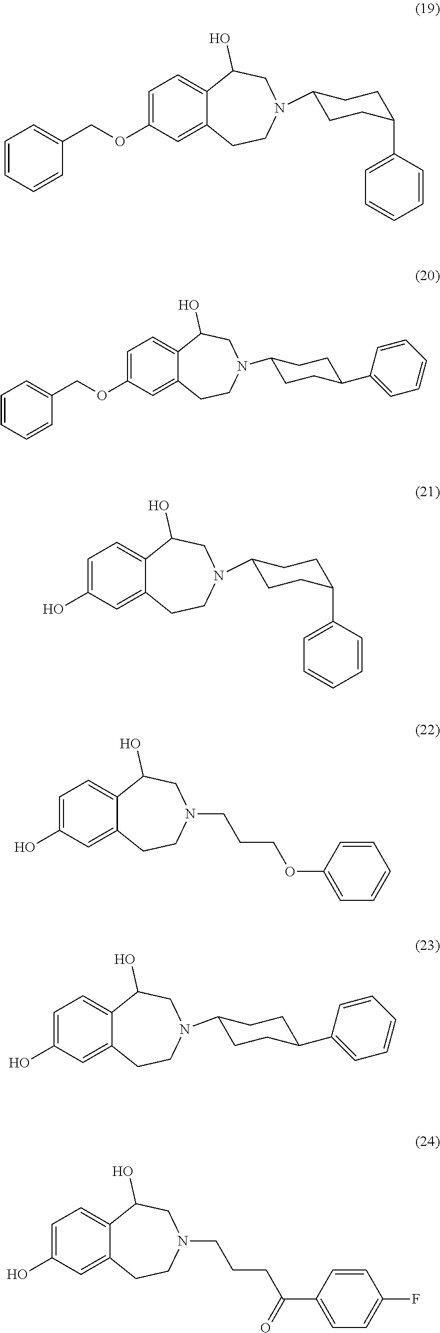 [SMILES: OC1CN(C2CCC(c3ccccc3)CC2)CCc2cc(OCc3ccccc3)ccc21 ; OC1CN(C2CCC(c3ccccc3)CC2)CCc2cc(OCc3ccccc3)ccc21 ; Oc1ccc2c(c1)CCN(C1CCC(c3ccccc3)CC1)CC2O ; Oc1ccc2c(c1)CCN(CCCOc1ccccc1)CC2O ; Oc1ccc2c(c1)CCN(C1CCC(c3ccccc3)CC1)CC2O ; O=C(CCCN1CCc2cc(O)ccc2C(O)C1)c1ccc(F)cc1]

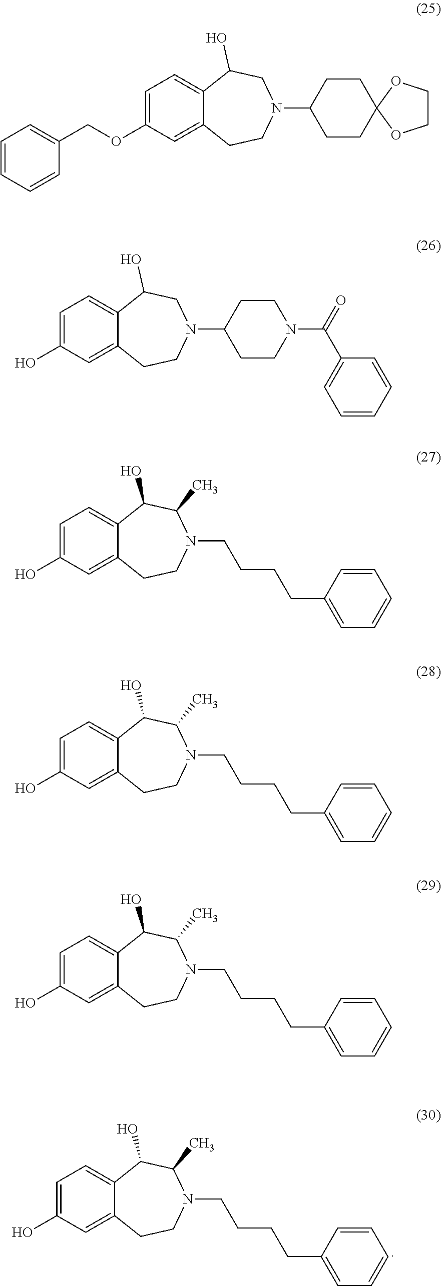 [SMILES: OC1CN(C2CCC3(CC2)OCCO3)CCc2cc(OCc3ccccc3)ccc21 ; O=C(c1ccccc1)N1CCC(N2CCc3cc(O)ccc3C(O)C2)CC1 ; C[C@@H]1[C@H](O)c2ccc(O)cc2CCN1CCCCc1ccccc1 ; C[C@H]1[C@@H](O)c2ccc(O)cc2CCN1CCCCc1ccccc1 ; C[C@H]1[C@H](O)c2ccc(O)cc2CCN1CCCCc1ccccc1 ; C[C@@H]1[C@@H](O)c2ccc(O)cc2CCN1CCCCc1ccccc1]

It is envisaged that compounds selected from the group comprising compounds according to formulas (1) to (30) exhibit a high affinity and/or selectivity to the NR2B subunit of the NMDA receptor as ascertainable as described in the appended Examples.

The compounds described herein can contain one or more double bonds and may thus give rise to cis/trans isomers as well as other conformational isomers. The present invention includes all such possible isomers as well as mixtures of such isomers.

Further, the compounds described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The formulas are either shown with or without a definitive stereochemistry at certain positions. If shown without a definitive stereochemistry the present invention includes all stereoisomers of the respective formula and solvates, hydrates, and pharmaceutically acceptable salts and esters thereof. Further, mixtures of stereoisomers as well as isolated specific stereoisomers are also included.

Unless specifically stated otherwise, differ compounds, groups or substituents denoted with Arabic numerals and such compounds, groups or substituents denoted with Roman numerals from each other, that is, compounds, groups or substituents are different compounds, groups or substituents.

### Immune-Mediated Inflammatory Diseases

The compounds as described herein are intended for treatment of “immune-mediated inflammatory diseases” or “IMID”. In its broadest sense, the term relates to diseases associated with common inflammatory pathogenesis mechanisms, which may result from, or be triggered by, a dysregulation of the normal immune response. IMIDs are typically caused, signified, or accompanied by dysregulation of the body's normal cytokine milieu and may cause acute or chronic inflammatory injury, sometimes severe, in any organ system. E.g., one causal manifestation of immune deregulation is the inappropriate expression of proinflammatory cytokines such as IL-1, IL-6 and TNF-α, as well as a Th1/Th2 cytokine disbalance leading to pathological changes.

The present inventors have surprisingly found that immune cells (in particular immune cells of the myeloid lineage, such as microglia) express the NR2B subunit of the NMDA receptor. It is thought that treatment of immune-mediated inflammatory diseases with the compounds according to the invention preferably results in a decrease in activation of immune cells involved in inflammatory pathways underlying IMID pathogenesis, and/or a reduced release of pro-inflammatory cytokines diminishing the recruitment and activation of other immune cells ultimatively resulting in a dampening of the immune-mediated tissue damage. In result, it is envisioned that treatment according to the invention preferably has an anti-inflammatory effect resulting in amelioration and/or remission of disease manifestations and/or symptoms. Having an “anti-inflammatory” effect in general means controlling and/or reducing any step of the inflammation cascade triggering and/or contributing to IMID pathogenesis.

It is particularly envisaged that the immune-inflammatory diseases to be treated according to the invention are characterized by one or more of the following features: (i) overexpession of proinflammatory cytokines, preferably IL-1, IL-6, IFN-γ, IL-17, and/or TNF-α; (ii) overexpression of autoantibodies, (iii) Th1/Th2 cytokine disbalance; (iv) Th17 disbalance, (v) changes in Treg function, (vi) autoimmune responses; and/or (vii) amelioration of disease symptoms by immunosuppressive therapies.

Means and methods to determine overexpression of cytokines such as IL-1, IL-6, IFNγ, IL-17, TNF-α are readily available in the art and have been reviewed e.g. by Methods Amsen et al. Mol Biol. 2009; 511: 107-142 and include, without limitation, quantification of cytokine mRNA by real-time quantitative PCR, and detection of cytokine proteins either by ELISA or in situ by immunohistochemistry.

Immune-inflammatory diseases may be accompanied by overexpression of disease-specific autoantibodies. The skilled person will readily acknowledge that specific autoantibodies indicate the presence of different IMIDs. E.g., in MS, autoantibodies against proteins of the myelin sheath, such as myelin basic protein (MBP), myelin oligodendrocyte glycoprotein (MOG), and proteolipid protein (PLP) may be present. Other examples are antinuclear antibodies, which are typically present in systemic lupus erythematosus, and rheumatoid factor or anti-cyclic citrullinated peptide (anti-CCP) antibodies, which are typically present in rheumatoid arthritis. A multitude of technologies are nowadays available for the detection of these autoantibodies, including, e.g. immunoblotting.

CD4+ T helper (Th) cells can be sugrouped as Th1 and Th2 cells. Th1 cells drive the type-1 pathway (“cellular immunity”) to fight viruses and other intracellular pathogens, eliminate cancerous cells, and stimulate delayed-type hypersensitivity (DTH) skin reactions. Th2 cells drive the type-2 pathway (“humoral immunity”) and up-regulate antibody production to fight extracellular organisms. Overactivation of either pattern can cause disease, and either pathway can down-regulate the other. The term “Th1/Th2 cytokine disbalance” is used herein to refer to a deviation of Th1 cytokine levels (IL-12, IFN-γ) and Th2 cytokine levels (IL-4, IL-6, IL-10)—as detectable e.g. by ELISA—in patients suffering from immune-mediated inflammatory diseases as compared to healthy controls.

Th17 cells are derived from naïve CD4+ precursor cells and secrete a characteristic profile of cytokines including IL-17A, IL-17F, GM-CSF, IL-21, and IL-22. Upon binding to their respective receptors, Th17 cytokines exhibit a variety of proinflammatory effects, including secretion of pro-inflammatory chemokines and cytokines. The term “Th17 disbalance” is used herein to refer to a disregulated and/or excessive Th17 cell differentiation or expansion; preferably accompanied by elevated levels of Th17 cytokines.

CD4+CD25+FOXP3+ Treg cells have been shown to suppress immune cells by a variety of cell contact dependent and independent mechanisms, including the production of cytokines such as IL-10, TGFrβ and IL-35, sequestration of cytokines essential for cell growth such and IL-2, surface expression of the immunosuppressive molecule cytotoxic T lymphocyte-associated antigen 4 (CTLA-4), and utilization of the perforin-granzyme pathway to kill activated targets or tumor cells. “Changes is Treg function”, i.e. the impairment of any of the aforementioned Treg mechanisms that generally act to induce immunologic tolerance, are often seen in IMIDs.

Besides the presence of autoantibodies, other autoimmune responses potentially seen in IMID patients include for example the presence of autoreactive cytotoxic T cells that target and attack particular cells or tissues of the body, the induction of cytokine-induced cell death, or complement activation resulting in inflammation and tissue damage.

A further indicator of IMIDs is the amelioration of disease symptoms by immunosuppressive therapies, such as, for instance, azathioprine, cyclosporine and corticosteroids such as prednisone.

Immune-mediated inflammatory diseases envisaged for treatment with the compounds of the invention include multiple sclerosis, rheumatoid arthritis, Crohn's disease, psoriasis, psoriatic arthritis, inflammatory bowel disease (IBD), ulcerative colitis (UC), systemic lupus erythematosus (SLE), Sjogren syndrome, ANCA-induced vasculitis, ankylosing spondylitis, anti-phospholipid syndrome, myasthenia gravis, Addison's disease, alopecia areata, ankylosing spondylitis, antiphospholipid antibody syndrome, antiphospholipid syndrome (primary or secondary), asthma, autoimmune gastritis, autoimmune hemolytic anemia, autoimmune hepatitis, autoimmune inner ear disease, autoimmune lymphoproliferative disease, autoimmune thrombocytopenic purpura, Balo disease, Behcet's disease, bullous pemphigoid, cardiomyopathy, celiac disease, Chagas disease, chronic inflammatory demyelinating polyneuropathy, cicatrical pemphigoid, cold agglutinin disease, degos disease, dermatitis hepatiformis, essential mixed cryoglobulinemia, Goodpasture's syndrome, Graves' disease, Guillain-Barre syndrome, Hashimoto's thyroiditis, idiopathic pulmonary fibrosis, idiopathic thrombocytopenia purpura, IgA nephropathy, juvenile arthritis, lichen planus, Meniere disease, mixed connective tissue disease, morephea, neuromyotonia, pemphigus vulgaris, pernicious anemia, polyarteritis nodosa, polychondritis, polymyalgia rheumatica, primary agammaglobulinemia, primary biliary cirrhosis, Raynaud disease (Raynaud phenomenon), Reiter's syndrome, relapsing polychondritis, rheumatic fever, Sjogren's syndrome, stiff-person syndrome (Moersch-Woltmann syndrome), Takayasu's arteritis, temporal arteritis (giant cell arteritis), uveitis, vasculitis, vitiligo, Wegener's granulomatosis, neuromyelitis optica, and isolated CNS-vasculitis.

Multiple sclerosis (MS) is an IMID particularly envisaged for treatment with the compounds of the present invention. The term includes the relapsing-remitting form of MS typically accompanied by exacerbations alternating with remissions during which partial or total recovery is observed. The remissions can last months or years. The exacerbations can occur spontaneously or be triggered by certain external factors, such as an infection, post-partum or certain vaccinations. The term further includes the primary progressive form of MS, wherein which evolves progressively without remissions, with the possibility of an evolutive plateau during which the disease does not progress. Contrary to the cyclic tendency, there are no clear exacerbations. The term further includes the secondary progressive form of MS which follows on from a remitting form and begins with attacks alternating with remissions, followed by a gradual progression of the disease without identifiable attacks.

It is envisaged that treatment with the compounds of the invention results in a reduced activation of immune cells in the patient. The term “immune cell” as used herein in its broadest sense generally comprises all lineages and types of leukocytes (i.e. white blood cells) derived from hematopoietic progenitor stem cells. Hematopoietic stem cells give rise to the myeloid leukocytes (including monocytes, macrophages such as microglia and Kupffer cells, neutrophils, eosinophils, basophils, and dendritic cells) and lymphoid leukocytes or lymphocytes (including B-cells, T-cells including CD4⁺ T helper cells (Th1, Th2, Th17, THαβ), regulatory T cells (T_{reg}), CD8⁺ Cytotoxic T cells, βδ T cells; natural killer (NK) cells and NKT cells), all of which are in principle envisaged as targets for the compounds as described herein in treatment of immune-mediated inflammatory diseases. Immune cells that are preferred targets for the compounds of the invention preferably express the NMDA receptor, in particular the NR2B receptor subunit.

The present inventors have surprisingly observed that immune cells, in particular microglia and monocytes, involved in pathogenesis of IMIDs such as multiple sclerosis, express the NMDA receptor, and in particular the NR2B subunit, and can be targeted with the compounds as described herein. Said compounds have been shown to act as NR2B-selective NMDA receptor antagonists, i.e. to bind specifically to the NR2B subunit of the NMDA receptor. Interestingly, targeting the NR2B subunit of the NMDA receptor on microglia and monocytes resulted in a marked downregulation of activation markers and cytokine expression and/or secretion. It is further envisaged that the NR2B subunit of the NMDA receptor is expressed on other immune cells, and in particular leukocytes of the myeloid lineage such as dendritic cells, other macrophages, neutrophils, eosinophils and basophils. Hence, without wishing to be bound by theory, treatment of immune-mediated inflammatory diseases is expected to result from reduced activation of the aforementioned immune-cells, which may in turn result in a decreased secretion of disease-promoting cytokines and/or other factors. A further effect of treatment with the compounds described herein may be the decreased activation of other effector cells involved in disease progression.

The reduced activation of immune cells is easily detectable e.g. by evaluating the expression of activation markers. Activation markers have been identified for many immune cells. For instance, in particular in case of monocytes, macrophages, microglia and dendritic cells, reduced activation of the immune cells is ascertainable by detection of specific activation markers including surface CD40, surface CD86, surface MHCII and/or surface CD80. Expression of these surface markers are readily ascertainable using routine FACS protocols and/or immunohistochemistry (IHC) or immunocytochemistry (ICC), e.g. as described in the appended examples. Expression of activation will generally be assessed using samples obtained from the treated patient will preferably be compared to samples obtained from the patient before begin of the treatment.

CD40, CD86, MHCII and CD80 are typically expressed on antigen presenting cells (APCs) and are generally known as co-stimulatory proteins involved in effector cell activation and survival (CD86, CD80), APC activation and survival (CD40) or antigen presentation (MHCII). Therefore, by inducing a downregulation of surface activation markers, the compounds described herein are thought to reduce effector cell activation and antigen presentation, thereby interrupting the inflammatory cascade associated with IMID disease onset and/or progression. Downregulation of other activation markers is however also envisaged and readily ascertainable by the skilled person in the art.

A further conceivable effect of IMID treatment with the compounds of the invention is the reduced proliferation of the target immune cells (e.g., microglia) and/or effector cells (e.g. T cells). Cell populations can be easily monitored and quantified using cell type-specific surface markes and FACS analysis.

As used herein “target (immune) cells” are in particular immune cells expressing the NR2B subunit of the NMDA receptor (such as microglia) whereas “effector (immune) cells” may comprise (immune) cells not expressing the NR2B subunit of the NMDA receptor subunit but being involved in IMID onset and/or progression. It is contemplated that said effector (immune) cells may be activated by target immune cells expressing the NR2B subunit of the NMDA receptor and/or respond to pro-inflammatory cytokines secreted by said target immune cells. Thus, treatment with the compounds of the invention advantageously reducing activation of target immune cells and/or secretion of pro-inflammatory cytokines is in turn also envisaged to indirectly reduce activation, survival, proliferation and/or recruitment of effector cells.

Further, treatment with the compounds of the invention is expected to result in reduced expression and/or release (secretion) of cytokines, for instance pro-inflammatory cytokines such as TNF-α, IFN-γ, IL1-beta and/or IL-6, from immune cells, in particular target immune cells. Release of cytokines will generally be assessed using samples obtained from the treated patient will preferably be compared to samples obtained from the patient before begin of the treatment. As described elsewhere herein, expression of cytokines is ascertainable for instance by quantitative Real Time PCR (qRT-PCT), whereas release of cytokines can be determined using, e.g., ELISAs or FACS-based methods.

### Patient

The term “patient” or “subject” as used herein refers to a human or non-human animal, generally a mammal. Particularly envisaged is a mammal, such as a rabbit, a mouse, a rat, a Guinea pig, a hamster, a dog, a cat, a pig, a cow, a goat, a sheep, a horse, a monkey, an ape or a human. Thus, the methods, uses and compounds described in this document are applicable to both human and veterinary disease. The subject to be treated may in particular be pre-diagnosed with an immune-inflammatory disease.

### Treatment

The term “treatment” in all its grammatical forms includes therapeutic or prophylactic treatment of immune-inflammatory disease. A “therapeutic or prophylactic treatment” comprises prophylactic treatments aimed at the complete prevention of clinical and/or pathological manifestations or therapeutic treatment aimed at amelioration or remission of clinical and/or pathological manifestations. The term “treatment” thus also includes the amelioration or prevention of immune-inflammatory diseases.

In the context with the present invention the term “therapeutic effect” in general refers to the desirable or beneficial impact of a treatment, e.g. amelioration or remission of the disease manifestations. The term “manifestation” of a disease is used herein to describe its perceptible expression, and includes both clinical manifestations, hereinafter defined as indications of the disease that may be detected during a physical examination and/or that are perceptible by the patient (i.e., symptoms), and pathological manifestations, meaning expressions of the disease on the cellular and molecular level. The therapeutic effect of treatment with the compounds of the invention can be assessed using routine methods known in the art. Additionally or alternatively it is also possible to evaluate the general appearance of the respective patient (e.g., fitness, well-being) which will also aid the skilled practitioner to evaluate whether a therapeutic effect has been elicited. The skilled person is aware of numerous other ways which are suitable to observe a therapeutic effect of the compounds of the present invention.

### Dose

Envisaged herein is the administration of a therapeutically effective amount of the compounds as described herein. By “therapeutically effective amount” is meant an amount of the compound that elicits a therapeutic effect as described herein. The exact dose of the compound will depend on the purpose of the treatment (e.g. remission maintenance vs. treatment of disease flares), and will be ascertainable by one skilled in the art using known techniques. Adjustments for route of administration, age, body weight, general health, sex, diet, time of administration, drug interaction and the severity of the condition may be necessary, and will be ascertainable with routine experimentation by those skilled in the art.

### Pharmaceutical Composition

Pharmaceutical compositions comprising the compounds of the invention for use in treatment of immune-mediated inflammatory diseases are also provided herein.

Preferably, the compound is present in the pharmaceutical composition in a therapeutically effective amount. The term “pharmaceutical composition” particularly refers to a composition suitable for administering to a human, i.e., a composition containing components which are pharmaceutically acceptable. However, compositions suitable for administration to an animal are also contemplated. The composition may preferably be sterile. Specifically, the term “pharmaceutically acceptable” may mean “approved by a regulatory agency or other generally recognized pharmacopoeia for use in animals, and more particularly in humans”. The pharmaceutical composition may further comprise a pharmaceutically acceptable excipient. The composition may also comprise further agents as described elsewhere herein. The term “excipient” includes fillers, binders, disintegrants, coatings, sorbents, antiadherents, glidants, preservatives, antioxidants, flavoring, coloring, sweeting agents, solvents, co-solvents, buffering agents, chelating agents, viscosity imparting agents, surface active agents, diluents, humectants. carriers, diluents, preservatives, emulsifiers, stabilizers or tonicity modifiers. As set our elsewhere herein, pharmaceutical compositions of the invention preferably comprise a therapeutically effective amount of the compound as described herein and can be formulated in various forms, e.g. in solid, liquid, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in a form which is particularly suitable for the desired method of administration.

Exemplary pharmaceutically acceptable carriers that are suitable for formulating the composition according the invention include (biodegradable) liposomes; microspheres made of the biodegradable polymer poly(D,L-lactic-coglycolic acid (PLGA), albumin microspheres; synthetic polymers (soluble); nanofibers, protein-DNA complexes; protein conjugates; erythrocytes; or virosomes. Various carrier based dosage forms comprise solid lipid nanoparticles (SLNs), polymeric nanoparticles, ceramic nanoparticles, hydrogel nanoparticles, copolymerized peptide nanoparticles, nanocrystals and nanosuspensions, nanocrystals, nanotubes and nanowires, functionalized nanocarriers, nanospheres, nanocapsules, liposomes, lipid emulsions, lipid microtubules/microcylinders, lipid microbubbles, lipospheres, lipopolyplexes, inverse lipid micelles, dendrimers, ethosomes, multicomposite ultrathin capsules, aquasomes, pharmacosomes, colloidosomes, niosomes, discomes, proniosomes, microspheres, microemulsions and polymeric micelles. Other suitable pharmaceutically acceptable carriers and excipients are inter alia described in Remington's Pharmaceutical Sciences, 15^{th} Ed., Mack Publishing Co., New Jersey (1991) and Bauer et al., Pharmazeutische Technologie, 5^{th} Ed., Govi-Verlag Frankfurt (1997).

### Additional Agents

The pharmaceutical composition of the present invention may further comprise one or more additional agents. Preferably, said agents are therapeutically effective for treatment of the particular immune-mediated inflammatory disease to be treated. The skilled person will readily be able to choose suitable additional agents. Exemplary additional agents envisaged for use within the pharmaceutical composition of the invention include, without limitation, corticosteroids, including prednisone and methylprednisolone, beta interferons, glatiramer acetate, dimethyl fumarate, fingolimod, teriflunomide, natalizumab, mitoxantrone, infliximab, etanercept, adalimumab, rituximab, abatacept, anakinra, alefacept, and/or efalizumab. Said agents thought to be particulary for treatment of multiple sclerosis, but may also be employed in treatment of other immune-mediated inflammatory diseases.

In view of the above, the present invention hence also provides a pharmaceutical composition for use in a method of treatment of immune-inflammatory diseases as specified elsewhere herein, the composition comprising corticosteroids, including prednisone and methylprednisolone, beta interferons, glatiramer acetate, dimethyl fumarate, fingolimod, teriflunomide, natalizumab, mitoxantrone, infliximab, etanercept, adalimumab, rituximab, abatacept, anakinra, alefacept, and/or efalizumab.

### Kit

It is also envisaged that the compound as described herein is can be used as part of a kit. Accordingly, in a further aspect, the present invention also relates to a kit comprising a compound as described herein for use in a method of treatment of immune-inflammatory diseases.

The kit may be a kit of two or more parts, and comprises the compound as described herein. The components of the kit may be contained in a container or vials. It is to be noted that all embodiments described in the context of the compound as described herein, the pharmaceutical composition comprising said compound and the methods of treatment can also be applied to the kit of the invention, mutatis mutandis.

It is envisaged that the kit may further comprise one or more additional agents suitable for treatment of immune-inflammatory diseases. The kit is thus intended for use in a method of treatment of immune-mediated inflammatory diseases. The skilled person will readily be able to provide additional agents depending on the disease to be treated. Exemplary suitable additional agents have been described in the context of the pharmaceutical composition and include, without limitation, corticosteroids, including prednisone and methylprednisolone, beta interferons, glatiramer acetate, dimethyl fumarate, fingolimod, teriflunomide, natalizumab, mitoxantrone, infliximab, etanercept, adalimumab, rituximab, abatacept, anakinra, alefacept, and/or efalizumab. It is envisaged that the additional agents are applied simultaneously, or sequentially, or separately with respect to the administration of the compound as described herein. The present invention further encompasses the application of the agents via different administration routes.

The kit is envisaged for treatment of immune-inflammatory diseases as described herein, and in particular multiple sclerosis.

### Administration

A variety of routes are applicable for administration of the compound as described herein of the present invention, including, but not limited to, orally, topically, transdermally, subcutaneously, intravenously, intraperitoneally, intramuscularly or intraocularly. However, any other route may readily be chosen by the person skilled in the art if desired.

The compounds may be provided in solid, liquid or gaseous form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in a form which is particularly suitable for the desired method of administration. The compounds may also be added to foods.

### Method of Treatment

Another aspect of the present invention is a method of treatment of immune-mediated inflammatory diseases in a subject in need thereof, comprising administering a therapeutically effective amount of a compound as described herein to said subject. The person skilled in the art will acknowledge that the embodiments described herein in the context the compound as described herein, the pharmaceutical composition and the kit of the present invention are applicable to the method of treatment, mutatis mutandis.

### NR2B-Selective NMDA Receptor Antagonists

As set out elsewhere herein, the compounds described herein are envisaged to act as NR2B-selective NMDA receptor antagonists for use in a method of treatment of multiple sclerosis.

The terms “NMDAR” and “NMDA receptor” are used interchangeably herein to refer to N-methyl-D-aspartate receptors comprising one or more subunits selected from NR1, NR2A, NR2B, NR2C, NR2D; NR3A and/or NR3B. An antagonist is a type of receptor ligand that blocks or reduces agonist-mediated responses without eliciting a response itself upon binding to a receptor. Thus, it is envisaged that the compounds described herein will block or reduce NMDA-mediated signalling in immune cells. It is further contemplated that the compounds do so by binding to the polyamine binding site of the NR2B subunit.

“NR2B” is also referred to as “GluN2B” herein. The compounds described herein bind selectively and with high affinity to the NR2B subunit of the receptor. It is hence thought that the compounds spare other NMDA receptors not comprising the NR2B subunit (which are abundant especially throughout the CNS) as well as other non-related receptors or surface molecules and therefore preferably do not evoke severe side effects as seen in other NMDA receptor antagonists. Affinity of the compounds described herein can be readily assessed as described in the appended examples.

Preferably, affinity of the compounds as described herein to the NR2B binding site of the NMDA receptor ranges from about 5 nM to 10 μm.

One preferred compound for use in treatment of immune-inflammatory diseases is the compound of the general formula

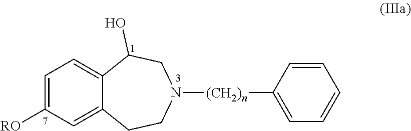

Said compound is also referred to as “WMS14-10” or “WMS” herein.

### Preparation of the Compounds

The compounds of the present invention may be prepared using a variety of processes well known by a person skilled in the art. Particularly suitable methods of preparing the compounds of the invention are disclosed in WO 2010/122134 A1. Accordingly, the compounds of the present invention wherein R₃ is not hydrogen may be prepared by a method comprising the steps of:
a) Preparation of a 2-(3-alkoxyphenyl)ethanol;
b) Esterification of the COOH group of an amino acid other than glycine;
c) Introduction of a protecting group to the nitrogen of the esterified amino acid of step b);
d) Nucleophilic substitution of the protected amino acid of step c) with 2-(3-alkoxyphenyl)ethanol of step a);
e) Alkaline hydrolysis of the alkyl ester of the amino acetate of step d) to the respective carboxylic acid;
f) Cyclisation of the carboxylic acid of step e) by a Friedel-Crafts acylation;
g) Reduction of the ketone product of step f) to the respective alcohol;
h) Elimination of the protecting group from the nitrogen and thereby obtaining the secondary amine of the compound of step g);
i) Introduction of a group R⁴ to the nitrogen of the compound of step h) by alkylation;
j) optionally hydrogenolytic elimination of the alkoxy group of step a) to receive a group R¹ that is hydrogen.

Unless otherwise indicated, R¹, R², R³, and R⁴ are as defined above.

In the method set out above, R³ is preferably selected from the group comprising hydrogen; linear or branched C₁-C₈-alkyl; C₂-C₈-alkenyl; C₃-C₈-cycloalkyl; C₆-C₁₀-aryl; C₄-C₁₀-cycloalkylalkyl wherein the cycloalkyl group has 3 to 6 carbon atoms and the alkyl group has 1 to 4 carbon atoms; C₇-C₁₄-arylalkyl wherein the aryl group has 6 to 10 carbon atoms and the alkyl group has 1 to 4 carbon atoms; linear or branched alkyl groups of the type —CₙH₂ₙ—U-D wherein n is 1 , 2, 3 or 4, U is selected from the group comprising O, CO, COO, CONH, S, guanidine and/or NH and D is selected from the group comprising H and/or C₁-C₃-alkyl; —CH₂—C₈H₄—X wherein X is selected from the group comprising OH, SH, C₁-C₃-alkyl and/or NH₂; —CH₂-imidazole; —CH₂-indole; —CH₂-(furanyl-3-yl); —CH₂-(pyridyl-3-yl) and/or —CH₂-(imidazolyl-3-yl).

The preparation of a 2-(3-alkoxyphenyl)ethanol of step a) preferably is a reaction of 1-bromo-4-methoxybenzene with ethylene sulfate to 2-(3-alkoxyphenyl)ethanol or a selective benzylation of 3-(2-hydroxyethyl)phenol to 2-(3-benzyloxyphenyl)ethanol.

The alkoxy group of a 2-(3-alkoxyphenyl)ethanol of step a) preferably is selected from the group comprising C₁-C₆-alkyloxy and/or benzyloxy, preferably is C₁-C₆-alkyloxy. A benzylation is preferred in embodiments when R¹ is benzyl or hydrogen and the benzyl group will be replaced by hydrogen in a later step.

The esterification of step b) preferably takes place using methanol and SOCl₂. Preferably, the esterification of step b) is carried out in the presence of an acid, preferably sulphuric acid.

The amino acid of step b) is an amino acid other than glycine since the method provides for compounds wherein R³ is not hydrogen. The amino acid preferably is a naturally occurring amino acid selected from the group comprising alanine, serie, threonine, arginine, lysine, aspartic acid, glutamic acid, asparagine, glutamine, phenylalanine, tyrosine, tryptophan, leucine, valine, isoleucine, cysteine, methionine, histidine and/or proline.

Preferably, if the amino acids have a functional group such as an hydroxyl or sulfhydryl group the functional groups are protected by a protecting group.

The amino acid of step b) can have a chiral center usually assigned by a prefix R or S, according to whether its configuration is right- or left-handed. Accordingly, the amino acid of step b) for example can be the R- or S-enantiomer of alanine. Advantageously, using an amino acid having a selected chiral center results in compounds having a selected chirality.

In step c) a group as a protecting group is introduced to the nitrogen of the esterified amino acid of step b) thereby replacing one of the hydrogen atoms of the primary amine by a protecting group. The protecting group preferably is selected from the group comprising sulfonic esters such as tosylates, mesylates, and trifluoromethanesulfonates (triflates). Preferably, step c) is a tosylation of the nitrogen of the esterified amino acid of step b). Preferably, the tosylation is carried out in the presence of a base, preferably selected from the group comprising pyridine, triethylamine and/or diisopropylamine.

In step d) a nucleophilic substitution of the protected amino acid, for example with a tosylate group, with 2-(3-alkoxyphenyl)ethanol of step a) is carried out. Preferably, the 905 nucleophilic substitution is a Mitsunobu reaction. The Mitsunobu Reaction allows the conversion of the primary alcohol group of the 2-(3-alkoxyphenyl)ethanol into an amine by nucleophilic substitution of the amino acid. The Mitsunobu reaction preferably is carried out using triphenylphosphine and diethyl azodicarboxylate (DEAD). A preferred solvent is tetrahydrofuran.

The alkaline hydrolysis of the alkyl ester of the amino acetate of step d) to the respective carboxylic acid in step e) preferably is carried out in the presence of LiOH. A preferred solvent is a mixture of tetrahydrofuran and water.

The cyclisation of the carboxylic acid by a Friedel-Crafts acylation in step f) preferably is carried out in the presence of 2,2,2-Trifluoroacetamide and stannous chloride (SnCl₂) or in the presence of phosphorpentoxide. A preferred solvent is dichloromethane. The Friedel-Crafts acylation allows the cyclisation from a reaction between the arene moiety and an acyl chloride of the carboxylic acid moiety of the compound.

The reduction of the cyclised ketone product of step f) to the respective alcohol in step g) preferably is carried out with a mild reducing agent such as LiAlH₄ or NaBH₄, preferably with NaBH₄. A preferred solvent for the reduction is methanol.

In step h) the protecting group such as a tosylate, mesylate, and trifluoromethanesulfonate from the nitrogen can be eliminated thereby obtaining the secondary amine of the compound wherein R⁴ is hydrogen. The elimination can be carried out using magnesium in methanol.

In compounds wherein R⁴ is not hydrogen but selected from the group comprising C₁-C₁₀-alkyl, —W and/or —Y—Z the group R⁴ is introduced to the nitrogen by an alkylation reaction. The alkylation can be carried out in the presence of help-bases. Preferred help-bases are selected from the group comprising sodium carbonate, sodium hydrogen carbonate, potassium carbonate, and/or potassium hydrogen carbonate.

Optionally, especially when the respective group R¹ should be hydrogene, the alkyoxy group of step a) can undergo hydrogenolytic elimination to receive a group R¹ that is hydrogen.

Preferably, the compounds of the present invention wherein R2 and R3 is hydrogen may be prepared by a method comprising the steps of:
1) Introduction of a protecting group to the nitrogen of 2-(3-alkoxyphenyl)ethane amine;
2) Nucleophilic substitution of the protected amine of step 1) with ethyl bromoacetate;
3) Alkaline hydrolysis of the ethyl ester of the amino acetate of step 2) to the respective carboxylic acid;
4) Cyclisation of the carboxylic acid of step 3) by a Friedel-Crafts acylation;
5) optionally Splitting the alkyl ether of the compound of step 4) and receiving the phenol;
6) optionally Benzylation of the phenol of step 5);
7) Reduction of the ketone product of step 4) or step 6) to the respective alcohol;
8) Elimination of the protecting group from the nitrogen and thereby obtaining the secondary amine of the compound of step 7);
9) Introduction of a group R4 to the nitrogen of the compound of step 8) by alkylation or acylation;
10) optionally hydrogenolytic elimination of the benzyl group to receive a group R1 that is hydrogen.

The alkoxy group of the 2-(3-alkoxyphenyl)ethane amine of step 1) preferably is selected from the group comprising C₁-C₆-alkyloxy and/or benzyloxy, preferably C—C₆-alkyloxy.

In step 1) a group as a protecting group is introduced to the nitrogen of the 2-(3-alkoxyphenyl)ethane amine, thereby replacing one of the hydrogen atoms of the primary amine group by a protecting group. The protecting group preferably is selected from the group comprising sulfonic esters such as tosylates, mesylates, and trifluoromethanesulfonates (triflates). Preferably, step 1) is a tosylation of the nitrogen. Preferably, the tosylation is carried out in the presence of a base, preferably selected from the group comprising pyridine, triethylamine and/or diisopropylamine.

In step 2) a nucleophilic substitution of the protected amine, for example with a tosylate group, of step 1) with ethyl bromoacetate is carried out. Preferably, the nucleophilic substitution is carried out using K₂CO₃.

In step 3), the alkaline hydrolysis of the ethyl ester of the amino acetate to the respective carboxylic acid preferably is carried out in the presence of sodium hydroxide in ethanol.

The cyclisation of the carboxylic acid by a Friedel-Crafts acylation in step 4) preferably is carried out in the presence of of phosphorpentoxide. A preferred solvent is dichloromethane. The Friedel-Crafts acylation allows the cyclisation from a reaction between the arene moiety and an acyl chloride of the carboxylic acid moiety of the compound.

Optionally, especially when the respective group R1 should be hydrogene, the alkyl ether of the compound of step 4) can become splitted to receive the phenol thereby having a group R1 that is hydrogen. The splitting of the alkyl ether preferably is carried out in the presence of aluminium chloride. A preferred solvent is dichloromethane.

However, to protect the hydroxyl group, in a next optional step the phenol of step 5) is benzylated. The benzylation of the phenol preferably is carried out using benzyl bromide and potassium carbonate.

The reduction of the cyclised ketone product of step 4) or step 6) to the respective alcohol in step 7) preferably is carried out with a mild reducing agent such as LiAlH₄ or NaBH4, preferably with NaBH₄. A preferred solvent for the reduction is methanol.

In step 8) the protecting group such as a tosylate, mesylate, and trifluoromethanesulfonate from the nitrogen can be eliminated thereby obtaining the secondary amine of the compound wherein R4 is hydrogen. The elimination can be carried out using magnesium in methanol.

For embodiments of the compound wherein R4 is not hydrogen but selected from the group comprising C₁-C₁₀-alkyl, —W and/or —Y—Z the group R4 is introduced to the nitrogen by an alkylation or an acylation reaction in step 9). The alkylation can be carried out in the presence of help-bases. Preferred help-bases are selected from the group comprising sodium carbonate, sodium hydrogen carbonate, potassium carbonate, and/or potassium hydrogen carbonate.

Optionally, especially when the respective group R1 should be hydrogen, the benzyl group introduced optionally in step 6) can undergo hydrogenolytic elimination to receive a group R1 that is hydrogen.

A better understanding of the present invention and of its advantages will be had from the following examples, offered for illustrative purposes only. The examples are not intended to limit the scope of the present invention in any way.

Unless stated otherwise, chemical reactions with substances sensitive towards hydrolysis or oxidation were carried out in water free glass equipment under inert gas. Nitrogen (Air Liquide, Düsseldorf, Germany) dried via a 5 Å molecular sieve was used as inert gas. For reactions at 4° C. cooling was achieved using a ice/water bath. For reactions at −78° C. cooling was achieved using a mixture of dry ice and acetone. If experiments were carried out at room or ambient temperature, that is, experiments were carried out at a temperature in the range of 18-25° C.

The solutions were used in per analysis quality. Tetrahydrofuran (THF) was distilled over sodium and benzophenone under nitrogen. Methanol (CH₃OH) was destilled over magnesium methanolate and stored on molecular sieve (0.3 nm). Dichlormethane (CH₂Cl₂) and dichlorethane were destilled over calcium hydride and stored on molecular sieve (0.4 nm).

A purification of compounds was carried out using flash column chromatography, a variant of column chromatography. Flash column chromatography was carried out using Merck silica gel 60 (40-63 μm). Pressure was achieved by nitrogen. The mobile phase, column diameter (Ø), filling level of silica gel, and volume of fractions was adjusted to experimental conditions and is given in detail in the respective examples. Further indicated is the retention factor (R_{f} value).

### EXAMPLES

### Example 1

### Preparation of 7-Methoxy-2,3,4,5-tetrahydro-1H-3-benzazepin-1-ol

### step 1.1 Preparation of N-[2-(3-Methoxyphenyl)ethyl]-4-toluenesulfonamide

2-(3-Methoxyphenyl)ethane-1-amine (5.0 g, 33.0 mmol) was dissolved in dry pyridine (60 mL). To the stirred solution 4-toluenesulfonylchloride (25.2 g, 132.2 mmol) was added and the mixture stirred for 1.5 h at room temperature. The mixture was treated with water (90 mL) and extracted with CHCl₃ (3×50 mL). The combined organic layer was washed with 5% HCl solution (4÷50 mL) and H₂O (3×50 mL), dried over Na₂SO₄ and evaporated. The residue was purified by flash column chromatography on silica gel (eluting with hexane: ethyl acetate 7:3 and 2% N,N-dimethylethaylmine, Ø 8 cm, volume of fraction 50 mL, R_{f}=0.28) to afford the titled compound as a light yellow solid.

### step 1.2 Preparation of 2-{N-[2-(3-Methoxyphenyl)ethyl]-N-(4-tosyl)-amino}acetate

N-[2-(3-Methoxyphenyl)ethyl]-4-toluenesulfonamide of step 1.1 (5.0 g, 16.4 mmol) was dissolved in acetone (85 mL) and to the stirred solution ethylbromacetate (4.2 g, 24.9 mmol) and K₂CO₃ (15.6 g, 113.0 mmol) was added. The mixture was heated for 20 h under reflux. The precipitate was filtered and the solvent was evaporated. The residue was purified by flash column chromatography on silica gel (eluting with hexane: ethyl acetate 7:3 and 2% N,N-dimethylethaylmine, Ø 8 cm, Volume of fraction 50 mL, R_{f}=0.51) to afford the titled compound as a light yellow solid.

### step 1.3 Preparation of 2-{N-[2-(3-Methoxyphenyl)ethyl]-N-(4-tosyl)amino} acetic acid

2-{N-[2-(3-Methoxyphenyl)ethyl]-N-(4-tosyl)-amino}acetate of step 1.2 (6.3 g, 16.3 mmol) was dissolved in NaOH (2.9 g, 72.5 mmol) and 50% ethanol (55 mL) and heated at reflux for 5 h. Ethanol was evaporated followed by the addition of H₂O (30 mL) to the solution.

After extraction with diethyl ether (3×30 mL) the aqueous phase was acidified with conc. HCl and back extracted three times with diethyl ether (30 mL). The organic layer was extracted with 5% NaHCO₃ solution (30 mL) and the aqueous phase was acidified with conc. HCl. After the acidified aqueous phase was extracted three times with diethyl ether (30 mL), the organic layer was dried with Na₂SO₄ and concentrated in vacuum.

The residue was purified by flash chromatography (CH₂Cl₂:CH₃OH 9.5:0.5 and 2% N,N-dimethylethanamine, Ø 8 cm, Volume of fraction 50 mL, R_{f}=0.62). The titled compound was obtained as a colourless solid.

### step 1.4 Preparation of 7-Methoxy-3-(4-tosyl)-2,3,4,5-tetrahydro-3-benzazepine-1-one

2-{N-[2-(3-Methoxyphenyl)ethyl]-N-(4-tosyl)amino} acetic acid (1.0 g, 2.76 mmol) from step 1.3 was dissolved in abs. dichlorethane (20 mL) and cooled under nitrogen to 0° C. Afterwards P₂O₅ (1.96 g, 13.8 mmol) was added to the solution and the mixture was allowed to stir for 24 h at 0° C. Afterwards to the suspension was added 3% NaOH to gain a pH-value between pH 13-14 and then extracted three times with dichloromethane (40 mL).

The dichloromethane phase was washed three times with water (50 mL), dried with Na₂SO₄ and concentrated in vacuum. The residue was purified by flash chromatography (n-hexane:ethyl acetate 7:3 and 2% N,N-dimethylethanamine, Ø 4 cm, volume of fraction 50 mL, R_{f}=0.23. The regioisomer 7-Methoxy-3-(4-tosyl)-2,3,4,5-tetrahydro-3-benzazepine-1-one was separated in ethanol by fractionated crystallization and obtained as a colourless solid.

### step 1.5 Preparation of 7-Methoxy-3-(4-tosyl)-2,3,4,5-tetrahydro-1H-3-benzazepine-1-ol

7-Methoxy-3-(4-tosyl)-2,3,4,5-tetrahydro-3-benzazepine-1-one (1.0 g, 2.90 mmol) resulting from step 1.4 was suspended in abs. CH₃OH (15 mL) and to the suspension NaBH₄ (0.230 g, 6.1 mmol) was added in several portions. After 2 h of stirring at room temperature the solvent was evaporated. The residue was combined with H₂O (30 mL) and extracted with CHCl₃ (4×30 mL). The organic layer was washed three times with H₂O (40 mL) and after drying with Na₂SO₄ the solvent was evaporated. The residue was purified by flash chromatography (CH₂Cl₂:diethyl ether 9.5:0.5, Ø 6 cm, volume of fraction 50 mL, R_{f}=0.15). The titled compound was obtained as a colourless solid.

### step 1.6 Preparation of 7-Methoxy-2,3,4,5-tetrahydro-1H-3-benzazepine-1-ol

7-Methoxy-3-(4-tosyl)-2,3,4,5-tetrahydro-1H-3-benzazepine-1-ol (1.85 g, 5.33 mmol) resulting from step 1.5 was dissolved in abs. CH₃OH (100 mL) together with magnesium turnings (2.83 g, 0.12 mol). The mixture was heated at reflux for 5 h and afterwards mixed with conc. H₂SO₄ (6.55 mL) under ice-cooling. The mixture was filtered to get rid of residues and the filtrate was adjusted with NaOH to an alkaline pH-value between pH=9-10. The water phase was extracted five times with CH₂Cl₂ (30 mL) and dried with Na₂SO₄. The solvent was evaporated. The residue was purified by flash chromatography (CH₂Cl₂:CH₃OH 19:1 and 2% NH₃, Ø 4.5 cm, volume of fraction 65 mL, R_{f}=0.11). The titled compound was obtained as a colourless solid.

### Example 2

### 7-Methoxy-3-(4-phenylbutyl)-2,3,4,5,-tertahydro-1H-3-benzazepine-1-ol

The compound 7-Methoxy-2,3,4,5-tetrahydro-1H-3-benzazepine-1-ol (100.0 mg, 0.52 mmol) synthesized according to step 1.6 was dissolved in CH₃CN (10 mL), TBAl (11.1 mg, 0.03 mmol), K₂CO₃ (575.0 mg, 4.16 mmol) and 1-chlor-4-phenylbutane (110.7 μL, 0.62 mmol). The suspension was heated at reflux for 48 h. Afterwards the K₂CO₃ was filtered and the solvent was evaporated. The residue was purified by flash chromatography (n-hexane: ethyl acetate 8:2 and 1% N,N-dimethylethanamine, Ø 2 cm, fraction size 10 mL, R_{f}=0, 15). The titled compound was obtained as a colourless solid.

### Example 3

### 7-Methoxy-3-octyl-2,3,4,5-tetrahydro-1H-3-benzazepine-1-ol

7-Methoxy-2,3,4,5-tetrahydro-1H-3-benzazepine-1-ol (100.0 mg, 0.52 mmol), synthesized according to step 1.6, was dissolved in CH₃CN (15 mL), subsequently mixed with K₂CO₃ (512 mg, 3.71 mmol) and 1-Bromoctan (80.5 μL, 0.55 mmol). The suspension was heated at reflux for 20 h. The precipitation was filtered and the solvent was removed in vacuum. The residue was purified by flash chromatography (n-hexane: ethyl acetate 7:3 and 1% N,N-dimethylethanamine, Ø 2 cm, fraction size 10 mL, R_{f}=0.34). The titled compound was obtained as a colourless solid.

### Example 4

### trans-7-Methoxy-3-(4-phenylcyclohexyl)-2,3,4,5-tetrahydro-1H-3-benzazepine-1-ol and cis-7-Methoxy-3-(4-phenylcyclohexyl)-2,3,4,5-tetrahydro-1H-3-benzazepine-1-ol

To a suspension of 7-Methoxy-2,3,4,5-tetrahydro-1H-3-benzazepine-1-ol (82.7 mg, 0.43 mmol), synthesized according to step 1.6, dichloroethane (2 mL) was added followed by the addition of phenylcyclohexanone (88.8 mg, 0.51 mmol), NaBH(OAc)₃ (136.1 mg, 0.64 mmol) and glacial acetic acid (36.6 μL, 0.64 mmol). The solution was stirred for 3 h at room temperature. Afterwards the solution was combined with saturated NaHCO₃ solution (10 mL) and H₂O (10 mL). The water phase was extracted with CH₂Cl₂. The combined organic layers were dried with Na₂SO₄ and the solvent was evaporated. The residue was purified by flash chromatography (n-hexane:ethyl acetate 7:3 and 1% N,N-dimethylethanamine, Ø 2 cm, fraction size 10 mL, R_{f}=0.15).

The diasteromers trans-7-Methoxy-3-(4-phenylcyclohexyl)-2,3,4,5-tetrahydro-1H-3-benzazepine-1-ol and cis-7-Methoxy-3-(4-phenylcyclohexyl)-2,3,4,5-tetrahydro-1H-3-benzazepine-1-ol in a ratio trans:cis=30:70, were obtained as a colourless oil.

### Example 5

### 7-Methoxy-3-(5-phenylpentyl)-2,3,4,5-tetrahydro-1H-3-benzazepine-1-ol

To the compound 7-Methoxy-2,3,4,5-tetrahydro-1H-3-benzazepine-1-ol (103.9 mg, 0.54 mmol), synthesized according to step 1.6, CH₃CN (10 mL), K₂CO₃ (431.2 mg, 3.12 mmol), TBAl (144.1 mg, 0.39 mmol) and 1-chlor-5-phenylpentan (104.5 μL, 0.58 mmol) were added. The suspension was heated at reflux for 39 h. Afterwards the K₂CO₃ was filtered and the solvent was evaporated. The residue was purified by flash chromatography (n-hexane: ethyl acetate 7:3 and 1% N,N-dimethylethanamine, Ø 3 cm, fraction size 30 mL, R_{f}=0.28). The titled compound was obtained as a colourless oil.

### Example 6

### 1-(4-Fluorphenyl)-4-(1-hydroxy-7-methoxy-2,3,4,5-tetrahydro-1H-3-benzazepine-3-yl)-butane-1-one

To the compound 7-Methoxy-2,3,4,5-tetrahydro-1H-3-benzazepine-1-ol (96.0 mg, 0.50 mmol), synthesized according to step 1.6, K₂CO₃ (552.8 mg, 4.0 mmol), CH₃CN (12 mL), TBAl (183.6 mg, 0.50 mmol) and 4-chloro-1-(4-fluorphenyl)butane-1-one (150.5 mg, 0.75 mmol) were added. The reaction mixture was heated at reflux for 72 h. The solvent was evaporated and the residue was purified by flash chromatography (n-hexane: ethyl acetate 6:4 and 1% N,N-dimethylethanamine, Ø 2 cm, fraction size 10 mL, R_{f}=0.09). The titled compound was obtained as a colourless resin.

### Example 7

### Preparation of 7-Methoxy-3-[4-(phenylsulfanyl)butyl]-2,3,4,5-tetrahydro-1H-3-benzazepi ne-1-ol

### Step 7.1 Preparation of 1-Chloro-3-(phenylsulfanyl)propane

Thiophenol sodium salt (0.75 g, 5.68 mmol) was dissolved in a mixture consisting of dioxane (4 mL) and H₂O (8 mL), followed by the addition of NaOH (0.23 g, 5.68 mmol). To the solution was added dropwise bromo-4-chlorpropane (0.67 mL, 6.80 mmol) and heated for 24 h at 60° C. The reaction mixture was diluted with H₂O (18 mL) and subsequently extracted with n-hexane (15 mL). The combined organic layers were treated with 2M NaOH (6×20 mL) and H₂O (2×20 mL). The organic phase was dried with Na₂SO₄ and the solvent was evaporated. The residue was purified by flash chromatography (n-hexane, Ø 3 cm, fraction size 10 mL, R_{f}=0.19). The titled compound was obtained as a pale yellow oil.

### step 7.2 Preparation of 1-Chloro-4-(phenylsulfanyl)butane

Thiophenol sodium salt (0.75 g, 5.68 mmol) was dissolved in a mixture consisting of dioxane (4 mL) and H₂O (8 mL), followed by the addition of NaOH (0.23 g, 5.68 mmol). To the solution was added dropwise bromo-4-chlorbutane (0.79 mL, 6.82 mmol) and heated for 24 h at 60° C. The reaction mixture was diluted with H₂O (18 mL) and subsequently extracted with n-hexane (15 mL). The combined organic layers were treated with 2M NaOH (6×20 mL) and H₂O (2×20 mL). The organic phase was dried with Na₂SO₄ and the solvent was evaporated. The residue was purified by flash chromatography (n-hexane: ethyl acetate 9.5:0.5, Ø 3 cm, fraction size 10 mL, R_{f}=0.75). The titled compound was obtained as a pale yellow oil.

### step 7.3 Preparation of 7-Methoxy-3-[4-(phenylsulfanyl)butyI]-2,3,4,5-tetrahydro-1H-3-benzazepine-1-ol

To 7-Methoxy-2,3,4,5-tetrahydro-1H-3-benzazepine-1-ol (87.7 mg, 0.45 mmol), synthesized according to step 1.6, CH₃CN (9 mL), TBAl (166.2 mg, 0.59 mmol), K₂CO₃ (497.5 mg, 3.60 mmol) and 1-chloro-4-(phenylsulfanyl)butane (117.8 mg, 0.59 mmol) resulting from step 7.2 were added. The suspension was heated at reflux for 8 h. Afterwards the K₂CO₃ was filtered and the solvent was evaporated. The residue was purified by flash chromatography (n-hexane: ethyl acetate 7:3 and 1% N,N-dimethylethanamine, Ø 3 cm, fraction size 30 mL, R_{f}=0.16). The titled compound was obtained as a pale yellow oil.

### Example 8

### 7-Methoxy-3-[3-(phenylsulfanyl)propyl]-2,3,4,5-tetrahydro-1H-3-benzazepine-1-ol

To 7-Methoxy-2,3,4,5-tetrahydro-1H-3-benzazepine-1-ol (100.6 mg, 0.52 mmol), synthesized according to step 1.6, (12 mL), TBAl (192.1 mg, 0.52 mmol), K₂CO₃ (574.9 mg, 4.16 mmol) and 1-chloro-3-(phenylsulfanyl)propane (125.1 mg, 0.67 mmol) resulting from step 7.1 were added. The suspension was heated at reflux for 48 h. Afterwards the K₂CO₃ was filtered and the solvent was evaporated. The residue was purified by flash chromatography (n-hexane:ethyl acetate 7:3 and 1% N,N-dimethylethanamin, Ø 3 cm, fraction size 30 mL, R_{f}=0.11). The titled compound was obtained as a colourless oil.

### Example 9

### Preparation of 3-{3-[2-(4-Fluorphenyl)-5,5-dimethyl-1,3-dioxan-2-yl]-propyl)-7-methoxy-2,3,4,5-tetrahydro-1H-3-benzazepine-1-ol

### step 9.1 Preparation of 2-(4-Fluorphenyl)-2-(3-chlorpropyl)-5,5-dimethyl-1,3-dioxane

4-Chloro-4′-fluorophenylbutane-l-one (1.0 g, 5.0 mmol), 2,2-dimethylpropane-1,3-diol (610 mg, 5.8 mmol) and p-toluenesulfonic acid (30 mg, 0.19 mmol) were dissolved in toluene (40 mL) and heated at reflux for 8 h at dean-Stark apparatus. The solution was washed with 8% NaHCO₃ solution (3×20 mL) and H₂O (2×30 mL). The organic phase was dried with Na₂SO₄ and the solvent was evaporated. The residue was purified by flash chromatography (n-hexane: ethyl acetate 9.5:0.5, Ø 4 cm, fraction size 20 mL, R_{f}=0.51). The titled compound was obtained as colourless solid.

### step 9.2 Preparation of 3-{3-[2-(4-Fluorphenyl)-5,5-dimethyl-1,3-dioxan-2-yl]-propyl)-7-methoxy-2,3,4,5-tetrahydro-1H-3-benzazepine-1-ol

The secondary amine 7-Methoxy-2,3,4,5-tetrahydro-1H-3-benzazepine-1-ol (380.0 mg, 1.32 mmol), synthesized according to step 1.6, and TBAl (487.6 mg, 1.32 mmol) were dissolved in CH₃CN (12 mL). Afterwards 2-(4-Fluorphenyl)-2-(3-chlorpropyl)-5,5-dimethyl-1,3-dioxane (200.0 mg, 1.04 mmol) from step 9.1 and K₂CO₃ (575.0 mg, 4.16 mmol) were added. The suspension was heated at reflux for 72 h. In the following step the insoluble residue was filtered and the solvent was evaporated. The residue was purified by flash chromatography (n-hexane: ethyl acetate 7:3 and 1% N,N-dimethylethanamine, Ø 2.5 cm, fraction size 10 mL, R_{f}=0.30). The titled compound was obtained as a colourless oil.

### Example 10

### Preparation of 1-(4-tert-Butylphenyl)-4-(1-hydroxy-7-methoxy-2,3,4,5-tetrahydro-1H-3-benzazepine-3-yl)butane-1-one

### step 10.1 Preparation of 2-(4-tert-Butylphenyl)-2-(3-chlorpropyl)-5,5-dimethyl-1,3-dioxan

4′-fert-Butyl 4-chlorophenylbutane-1-one (1.14 g, 4.78 mmol), 2,2-dimethylpropane-1,3-diol (600 mg, 5.74 mmol) and p-toluenesulfonic acid (30 mg, 0.19 mmol) were dissolved in toluene (40 mL) and heated at reflux for 4 h at dean-Stark apparatus. The solution was washed with 8% NaHCO₃ solution (3×20 mL) and H₂O (2×30 mL). The organic phase was dried with Na₂SO₄ and the solvent was evaporated. The residue was purified by flash chromatography (n-hexane:ethyl acetate 9.5:0.5, Ø 4 cm, fraction size 20 mL, R_{f}=0.31). The titled compound was obtained as colourless solid.

### step 10.2 Preparation of 3-{3-[2-(4-tert-Butyl-phenyl)-5,5-dimethyl-1,3-dioxan-2-yl]-propyl}-7-methoxy-2,3,4,5-tetrahydro-1H-3-benzazepine-1-ol

To the compound 7-Methoxy-2,3,4,5-tetrahydro-1H-3-benzazepine-1-ol (161.7 mg, 0.50 mmol), synthesized according to step 1.6, and TBAl (184.7 mg, 0.50 mmol) in CH₃CN (5 mL) was added 2-(4-tert-Butylphenyl)-2-(3-chlorpropyl)-5,5-dimethyl-1,3-dioxan resulting from step 10.1 and K₂CO₃ (226.6 mg, 1.64 mmol). The suspension was heated at reflux for 50 h. Finally the insoluble solid was filtered and the solvent was evaporated. The residue was purified by flash chromatography (n-hexane: ethyl acetate 7:3 and 1% N,N-dimethylethanamine, Ø 2 cm, fraction size 10 mL, R_{f}=0.21). The titled compound was obtained as a colourless oil.

### step 10.3 Preparation of 1-(4-tert-Butylphenyl)-4-(1-hydroxy-7-methoxy-2,3,4,5-tetrahydro-1H-3-benzazepine-3-yl)butane-1-one

To 3-{3-[2-(4-tert-Butyl-phenyl)-5,5-dimethyl-1,3-dioxan-2-yl]-propyl}-7-methoxy-2,3,4,5-tetrahydro-1H-3-benzazepine-1-ol (190.0 mg, 0.39 mmol), synthesized according to step 10.2, diethyl ether (5 mL) was added, followed by the addition of 1M HCl (5 mL). The suspension was stirred for 16 h at room temperature. Afterwards saturated NaHCO₃ solution (3×5 mL) was added. The aqueous phase was extracted three times with CH₂CL₂ (5 mL) and the combined organic layers were dried with Na₂SO₄). The solvent was evaporated and the residue was purified by flash chromatography (n-hexane: ethyl acetate 5:5 and 1% N,N-dimethylethanamine, Ø 2 cm, fraction size 10 mL, R_{f}=0.20). The titled compound was obtained as a colourless oil.

### Example 11

### Preparation of 7-Benzyloxy-2,3,4,5-tetrahydro-1H-3-benzazepine-1-ol

### step 11.1 Preparation of 7-Hydroxy-3-(4-tosyl)-2,3,4,5-tetrahydro-3-benzazepine-1-one

The 7-Methoxy-3-(4-tosyl)-2,3,4,5-tetrahydro-3-benzazepine-1-one (1.06 g, 3.07 mmol), synthesized according to step 1.4, was dissolved in CH₂Cl₂ (50 mL) followed by the addition of AlCl₃ (4.91 g, 36.8 mmol). The suspension was heated at reflux for 23 h. The suspension was mixed with water (60 mL) under ice-cooling and stirred for an additional 1 h. The aqueous phase was extracted with a mixture of CH₂Cl₂ and CH₃OH ((8:2) 3×30 mL). The combined organic layers were dried with Na₂SO₄ and the solvent was evaporated. The residue was purified by flash chromatography (n-hexane:ethyl acetate 7:3, Ø 3 cm, fraction size 30 mL, R_{f}=0.14). The titled compound was obtained as a colourless solid.

step 11.2 Preparation of 7-Benzyloxy-3-(4-tosyl)-2,3,4,5-tetrahydro-3-benzazepine-1-one

7-Hydroxy-3-(4-tosyl)-2,3,4,5-tetrahydro-3-benzazepine-1-one (0.61 g, 1.83 mmol) synthesized according to step 11.1 was dissolved in acetone (60 mL), followed by the addition of K₂CO₃ (1.01 g, 7.32 mmol) and benzylbromide (0.38 g, 2.20 mmol). The reaction mixture was heated at reflux for 4 h. Afterwards the excess of K₂CO₃ was filtered and the solvent was evaporated. The residue was purified by flash chromatography (n-hexane:ethyl acetate 7:3, Ø 5.5 cm, fraction size 65 mL, R_{f}=0.31). The titled compound was obtained as a colourless solid.

### step 11.3 Preparation of 7-Benzyloxy-3-(4-tosyl)-2,3,4,5-tertahydro-1H-3-benzazepine-1-ol

7-Benzyloxy-3-(4-tosyl)-2,3,4,5-tetrahydro-3-benzazepine-1-one (0.40 g, 0.94 mmol) synthesized according to step 11.2 was suspended in abs. CH₃OH (25 mL) and to the suspension NaBH₄ (0.21 g, 5.64 mmol) was added in several portions. After stirring 6 h at room temperature to the reaction mixture was added H₂O (30 mL) and finally four times extracted with CHCl₃ (30 mL). The organic layer was washed three times with H₂O (40 mL) and after drying with Na₂SO₄ the solvent was evaporated. The residue was purified by flash chromatography (n-hexane:ethyl acetate 5:5, Ø 3 cm, fraction size 10 mL, R_{f}=0.59). The titled compound was obtained as a colourless solid.

### step 11.4 Preparation of 7-Benzyloxy-2,3,4,5-tetrahydro-1H-3-benzazepine-1-ol

To 7-Benzyloxy-3-(4-tosyl)-2,3,4,5-tertahydro-1H-3-benzazepine-1-ol (114.1 mg, 0.27 mmol) synthesized according to step 11.3 in CH₃OH (10 mL) was added Mg (144.2 mg, 5.93 mmol). The mixture was heated at reflux for 5 h, followed by the addition of concentrated H₂SO₄ (0.33 mL) under ice-cooling.

Insoluble material was filtered and the solution was adjusted with NaOH to pH 9. The aqueous phase was extracted five times with CH₂Cl₂ (15 mL) and the organic layer was dried with Na₂SO₄ and removed by evaporation. The residue was purified by flash chromatography (CH₂Cl₂:CH₃OH 9.5:0.5 and 2% NH₃, ● 2 cm, fraction size 10 mL, R_{f}=0.1 1).

The titled compound was obtained as a colourless solid.

### Example 12

### 7-Benzyloxy-3-(4-phenylbutyl)-2,3,4,5-tetrahydro-1H-3-benzazepine-1-ol

To 7-Benzyloxy-2,3,4,5-tetrahydro-1H-3-benzazepine-1-ol (42.9 mg, 0.16 mmol) synthesized according to step 11.4 in CH₃CN (5 mL) was added TBAl (59.1 mg, 0.16 mmol), K₂CO₃ (176.9 mg, 1.28 mmol) and 1-chloro-4-phenylbutane (39.5 μL, 0.24 mmol). The suspension was heated at reflux for 72 h. Afterwards the K₂CO₃ was filtered and the solvent was evaporated. The residue was purified by flash chromatography (n-hexane:ethyl acetate 7:3 and 1% N,N-dimethylethanamine, Ø 2 cm, fraction size 10 mL, Rf=0.30). The titled compound was obtained as a colourless oil.

### Example 13

### Preparation of 7-(Benzyloxy)-3-[3-(phenylsulfonyl)propyl]-2,3,4,5-tetrahydro-1H-3-benzazepine-1-ol

### step 13.1 Preparation of 1-Chlor-3-(phenylsulfonyl)propane

To 1-Chlor-3-(phenylsulfanyl)propane (132.0 mg, 0.71 mmol) synthesized according to step 7.1 was added glacial acetic acid (5 mL) and heated at reflux for 30 min. After removing the heating the dropwise addition of 30% H₂O₂ solution (213.0 μL, 2.41 mmol) followed. Afterwards the solution was heated at reflux for 30 min. To the reaction mixture H₂O (10 mL) was added and extracted three times with toluene (10 mL). The organic layer was washed with 10% Na₂S₂O₃ solution, dried with Na₂SO₄ and the solvent was evaporated. The residue was purified by flash chromatography (n-hexane:ethyl acetate 8:2, Ø 2 cm, fraction size 10 mL, R_{f}=0.14). The titled compound was obtained as pale yellow oil.

### step 13.2 Preparation of 7-(Benzyloxy)-3-[3-(phenylsulfonyl)propyl]-2,3,4,5-tetrahydro-1H-3-benzazepine-1-ol

To 7-Benzyloxy-2,3,4,5-tetrahydro-1H-3-benzazepine-1-ol (278.3 mg, 1.03 mmol) synthesized according to step 11.4 in CH₃CN (16 mL) were added TBAl (379.5 mg, 1.13 mmol), K₂CO₃ (569.4 mg, 4.12 mmol) and 1-chloro-3-(phenylsulfonyl)propane (234.4 mg, 1.13 mmol) from step 13.1. The suspension was heated at reflux for 72 h. In the following step the insoluble residue was filtered and the solvent was evaporated.

The residue was purified by flash chromatography (n-hexane:ethyl acetate 5:5 and 1% N,N-dimethylethanamine, Ø 4 cm, fraction size 10 mL, R_{f}=0.15). The titled compound was obtained as pale yellow oil.

### Example 14

### 7-(Benzyloxy)-3-[3-(phenylsulfanyl)propyl]-2,3,4,5-tetrahydro-1H-3-benzazepine-1-ol

To 7-Benzyloxy-2,3,4,5-tetrahydro-1H-3-benzazepine-1-ol (300.0 mg, 1.12 mmol) resulting from step 11.4 in CH₃CN (16 mL) were added TBAl (413.7 mg, 1.12 mmol), K₂CO₃ (619.1 mg, 4.48 mmol) and 1-chloro-3-(phenylsulfanyl)propane from step 7.1 (249.0 mg, 1.34 mmol). The suspension was heated at reflux for 48 h.

In the following step the insoluble residue was filtered and the solvent was evaporated. The residue was purified by flash chromatography (n-hexane:ethyl acetate 7:3 and 1% N,N-dimethylethanamine, Ø 2 cm, fraction size 10 mL, R_{f}=0.25). The titled compound was obtained as colourless oil.

### Example 15

### 7-(Benzyloxy)-3-(3-[2-(4-fluorphenyl)-5,5-dimethyl-1,3-dioxan-2-yl[propyl)-2,3,4,5-tetrahydro-1H-3-benzazepine-1-ol

To 7-benzyloxy-2,3,4,5-tetrahydro-1H-3-benzazepine-1-ol (292.0 mg, 1.09 mmol) resulting from step 11.4 in CH₃CN (16 mL) was added TBAl (403.0 mg, 1.09 mmol), K₂CO₃ (603.0 mg, 4.36 mmol) and 2-(4-Fluorphenyl)-2-(3-chlorpropyl)-5,5-dimethyl-1,3-dioxane (370.0 mg, 1.30 mmol) from step 9.1. The suspension was heated at reflux for 62 h. In the following step the insoluble residue was filtered and the solvent was evaporated. The residue was purified by flash chromatography (n-hexane:ethyl acetate 6:4 and 1% N,N-dimethylethanamine, Ø 3 cm, fraction size 10 mL, R_{f}=0.36). The titled compound was obtained as colourless oil.

### Example 16

### trans-7-(Benzyloxy)-3-(4-phenylcyclohexyl)-2,3,4,5-tetrahydro-1H-3-benzazepine-1-ol and

### cis-7-(Benzyloxy)-3-(4-phenylcyclohexyl)-2,3,4,5-tetrahydro-1H-3-benzazepine-1-ol

To the secondary amine 7-Benzyloxy-2,3,4,5-tetrahydro-1H-3-benzazepine-1-ol (349.7 mg, 1.30 mmol) synthesized according to step 11.4 and 4-phenylcyclohexanone (272.0 mg, 1.56 mmol) dissolved in dichlorethane (25 mL), glacial acetic acid (104.6 μL) was added. Several portions of NaBH(OAc)₃ (495.8 mg, 2.34 mmol) were added to the reaction mixture followed by stirring for 16 h. To the mixture saturated NaHCO₃ solution (20 mL) and H₂O (20 mL) were added. The phases were separated and the aqueous phase was extracted three times with CH₂Cl₂ (40 mL). The combined organic layers were dried with Na₂SO₄ and the solvent was evaporated. The residue was purified by flash chromatography (n-hexane:ethyl acetate 9:1 and 1% N,N-dimethylethanamine, Ø 3 cm, fraction size 10 mL, R_{f}(trans-7-(Benzyloxy)-3-(4-phenylcyclohexyl)-2,3,4,5-tetrahydro-1H-3-benzazepine-1-ol)=0.30, R_{f}(cis-7-(Benzyloxy)-3-(4-phenylcyclohexyl)-2,3,4,5-tetrahydro-1H-3-benzazepine-1-ol)=0.36).

trans-7-(Benzyloxy)-3-(4-phenylcyclohexyl)-2,3,4,5-tetrahydro-1H-3-benzazepine-1-ol was obtained as a colourless solid. cis-(Benzyloxy)-3-(4-phenylcyclohexyl)-2,3,4,5-tetrahydro-1H-3-benzazepine-1-ol was obtained as a colourless oil.

### Example 17

### 7-(Benzyloxy)-3-[(3-phenylbenzyl)-methyl]-2,3,4,5-tetrahydro-1H-3-benzazepine-1-ol

To 7-Benzyloxy-2,3,4,5-tetrahydro-1H-3-benzazepine-1-ol (250.1 mg, 0.93 mmol) from step 11.4 in CH₃CN (25 mL), K₂CO₃ (513.7 mg, 3.7 mmol) and 3-phenylbenzyl-bromide (299.0 mg, 1.21 mmol) were added. The suspension was heated at reflux for 72 h. In the following step K₂CO₃ was filtered and the solvent was evaporated. The residue was purified by flash chromatography (n-hexane:ethyl acetate 7:3 and 1% N,N-dimethylethanamine, Ø 3 cm, fraction size 10 mL, R_{f}=0.29). The titled compound was obtained as pale yellow oil.

### Example 18

### 7-Benzyloxy-3-(1,4-dioxaspiro[4.5]decan-8-yl)-2,3,4,5-tetrahydro-1H-3-benzazepine-1-ol

To the secondary amine 7-Benzyloxy-2,3,4,5-tetrahydro-1H-3-benzazepine-1-ol (101.5 mg, 0.38 mmol) from step 11.4 and cyclohexane-1,4-dion-monoethylenketal (88.4 mg, 0.57 mmol) in CH₂Cl₂ (3 mL) was added NaBH(OAc)₃ (120.8 mg, 0.57 mmol). The reaction mixture was stirred for 16 h and finally mixed with H₂O (10 mL). The aqueous phase was washed three times with CH₂Cl₂ (10 mL) and the combined organic layers dried with Na₂SO₄.

The solvent was evaporated and residue was purified by flash chromatography (n-hexane:ethyl acetate 5.5:4.5 and 1% N,N-dimethylethanamine, Ø 2 cm, fraction size 10 mL, R_{f}=0.14). The titled compound was obtained as colourless resin.

### Example 19

### 3-(4-Phenylbutyl)-2,3,4,5-tetrahydro-1H-3-benzazepine-1,7-diol

To 7-Benzyloxy-3-(4-phenylbutyl)-2,3,4,5-tetrahydro-1H-3-benzazepine-1-ol (39.0 mg, 0.10 mmol) from the example 12 in CH₃OH (5 mL) was added Pd/C (37.0 mg, 10%). The suspension was stirred under H₂-atmosphere for 30 min at room temperature. The catalysator was filtered and the solvent was removed. The residue was purified by recrystallisation in diisopropyl ether. The titled compound was obtained as pale yellow oil.

### Example 20

### Preparation of N-Benzyl-2-[7-(benzyloxy)-1-hydroxy-2,3,4,5-tetrahydro-1H-3-benzazepine-3-yl]-N-methylacetamide

### step 20.1 Preparation of N-Benzyl-2-chloro-N-methylacetamide

N-benzyl-N-methylamine (1.0 g, 8.25 mmol) was dissolved in toluene (25 mL) and triethylamine (1.1 mL, 8.25 mmol) was added. Chloracetylchloride (657 pL, 8.25 mmol) in toluene (5 mL) was added dropwise to the solution. The reaction mixture was heated to 35° C. and stirred for 10 h. After addition of H₂O (15 mL) the water phase was extracted three times with diethyl ether (10 mL). The combined organic layers were treated with Na₂SO₄ solution (3×20 mL) and dried with K₂CO₃. The solvent was evaporated. And the residue was purified by flash chromatography (n-hexane:ethyl acetate 5:5, Ø 4 cm, fraction size 10 mL, R_{f}=0.48). The titled compound was obtained as colourless oil.

### step 20.2 Preparation of N-Benzyl-2-[7-(benzyloxy)-1-hydroxy-2,3,4,5-tetrahydro-1H-3-benzazepine-3-yl]-N-methylacetamide

To 7-Benzyloxy-2,3,4,5-tetrahydro-1H-3-benzazepine-1-ol (250.0 mg, 0.93 mmol) from step 11.4 , N-Benzyl-2-chloro-N-methylacetamide (274.6 mg, 1.39 mmol) resulted from step 20.1 TBAl (513.0 mg, 1.39 mmol) and K₂CO₃ (514.1 mg, 3.72 mmol) were added CH₃CN (16 mL). The suspension was heated at reflux for 50 h. The insoluble precipitation was filtered and the solvent was evaporated. The residue was purified by flash chromatography (n-hexane:ethyl acetate 2:8 and 1% N,N-dimethylethanamine, Ø 3 cm, fraction size 10 mL, R_{f}=0.20). The titled compound was obtained as pale yellow oil.

### Example 21

### N-Benzyl-2-(1,7-dihydroxy-2,3,4,5-tetrahydro-1H-3-benzazepine-3-yl)-N-methylacetamide

To N-Benzyl-2-[7-(benzyloxy)-1-hydroxy-2,3,4,5-tetrahydro-1H-3-benzazepine-3-yl]-N-methylacetamide (307.1 mg, 0.71 mmol), from example 20, in abs. CH₃OH (30 mL) was added Pd/C (61.4 mg, 10%). The suspension was stirred under H₂-atmosphere (1 bar) for 1 h at room temperature. The catalysator was filtered and the solvent was removed. The titled compound was obtained as colourless solid.

### Example 22

### cis-3-(4-Phenylcyclohexyl)-2,3,4,5-tetrahydro-1H-3-benzazepine-1,7-diol

To cis-7-(Benzyloxy)-3-(4-phenylcyclohexyl)-2,3,4,5-tetrahydro-1H-3-benzazepine-1-ol (340,2 mg, 0.80 mmol), from example 16, in abs. CH₃OH (30 mL) was added Pd/C (200.0 mg, 10%). The suspension was stirred under H₂-atmosphere (1 bar) for 2 h at room temperature. The catalysator was removed by Celite® 535 filtration then the solvent was removed. The residue was purified by flash chromatography (n-hexane:ethyl acetate 6:4 and 1% N,N-dimethylethanamine, Ø 2 cm, fraction size 10 mL, R_{f}=0.19). The titled compound was obtained as colourless solid.

### Example 23

### 7-Benzyloxy-3-(3-phenoxypropyl)-2,3,4,5-tetrahydro-1H-3-benzazepine-1-ol

The suspension of the secondary amine 7-benzyloxy-2,3,4,5-tetrahydro-1H-3-benzazepine-1-ol (202.8 mg, 0.75 mmol) from step 11.4, 3-phenoxypropylbromide (178.2 μL, 1.13 mmol) and K₂CO₃ (518.2 mg, 3.75 mmol) in CH₃CN (25 mL) were heated at reflux for 50 h. The insoluble precipitation was filtered and the solvent was evaporated. The residue was purified by flash chromatography (n-hexane:ethyl acetate 7:3 and 1% N,N-dimethylethanamine, Ø 3 cm, fraction size 10 mL, R_{f}=0.25). The titled compound was obtained as pale yellow oil.

### Example 24

### 4-(7-Benzyloxy-1-hydroxy-2,3,4,5-tetrahydro-1H-3-benzazepine-3-yl)-1-(4-tert-butylphenyl)butane-1-one

To the compound 7-(Benzyloxy)-3-{3-[2-(4-tert-butylphenyl)-5,5-dimethyl-1,3-dioxan-2-yl]propyl}-2,3,4,5-tetrahydro-1H-3-benzazepine-1-ol (115.0 mg, 0.21 mmol) dissolved in diethyl ether (5 mL) was added 1M HCl (5 mL). The suspension was stirred for 16 h at room temperature, followed by the addition of saturated NaHCO₃-solution (5 mL). The water phase was extracted with CH₂Cl₂ (3×5 mL) and dried with Na₂SO₄. The solvent was evaporated and the residue was purified by flash chromatography (n-hexane:ethyl acetate 5:5+1% N,N-dimethylethanamine, 0 2 cm, fraction size 10 mL, R_{f}=0.24). The titled compound was obtained as a colourless oil.

Example 25

### 3-(3-Phenoxypropyl)-2,3,4,5-tetrahydro-1H-3-benzazepine-1,7-diol

To 7-benzyloxy-3-(3-phenoxypropyl)-2,3,4,5-tetrahydro-1H-3-benzazepine-1-ol (250.6 mg, 0.62 mmol), from example 23, in abs. CH₃OH (20 mL) was added Pd/C (202.0 mg, 10%) The suspension was stirred under H₂-atmosphere (1 bar) for 2 h at room temperature. The catalysator was removed by Celite® 535 filtration then the solvent was removed. The residue was purified by flash chromatography (n-hexane:ethyl acetate 2:8 and 1% N,N-dimethylethanamine, Ø 2 cm, fraction size 10 mL, R_{f}=0.24). The titled compound was obtained as yellow resin.

### Example 26

### trans-3-(4-Phenylcyclohexyl)-2,3,4,5-tetrahydro-1H-3-benzazepine-1,7-diol

To trans-7-(benzyloxy)-3-(4-phenylcyclohexyl)-2,3,4,5-tetrahydro-1H-3-benzazepine-1-ol (74.5 mg, 0.17 mmol), from example 16, dissolved in abs. THF (5 mL) was added Pd/C (40.0 mg, 10%). The suspension was stirred under H₂-atmosphere (1 bar) for 2 h at room temperature. The catalysator was removed by Celite® 535 filtration then the solvent was removed. The residue was purified by flash chromatography (n-hexane:ethyl acetate 5:5 and 1% N,N-dimethylethanamin, Ø 2 cm, fraction size 10 mL, R_{f}=0.18). The titled compound was obtained as a colourless solid.

### Example 27

### Preparation of 4-(1,7-Dihydroxy-2,3,4,5-tetrahydro-1H-3-benzazepine-3-yl)-1-(4-fluorphenyl)butane-1-one

### step 27.1 Preparation of 4-(7-Benzyloxy-1-hydroxy-2,3,4,5-tetrahydro-1H-3-benzazepine-3-yl)-1-(4-fluorphenyl)butan-1-on

To 7-(Benzyloxy)-3-(3-[2-(4-fluorphenyl)-5,5-dimethyl-1,3-dioxan-2-yl]propyl)-2,3,4,5-tetrahydro-1H-3-benzazepine-1-ol (144.2 mg, 0.28 mmol), from example 15, was added diethyl ether (4 mL) and 1M HCl (4 mL). The suspension was stirred for 16 h at room temperature and mixed with saturated NaHCO₃ solution (8 mL). The aqueous phase was extracted with CH₂Cl₂ (3×10 mL) and the combined organic layers were dried with Na₂SO₄. The solvent was evaporated and the residue was purified by flash chromatography (n-hexane:ethyl acetate 6:4 and 1% N,N-dimethylethanamine, Ø 2.5 cm, fraction size 10 mL, R_{f}=0.20). The titled compound was obtained as colourless oil.

### step 27.2 Preparation of 4-(1,7-Dihydroxy-2,3,4,5-tetrahydro-1H-3-benzazepine-3-yl)-1-(4-fluorphenyl)butane-1-one

To the 4-(7-Benzyloxy-1-hydroxy-2,3,4,5-tetrahydro-1H-3-benzazepine-3-yl)-1-(4-fluorphenyl)butane-1-one (60.9 mg, 0.14 mmol), from the step 27.1, in abs. CH₃OH (6 mL) was added Pd/C (12.2 mg, 10%). The suspension was stirred under H₂-atmosphere (1 bar) for 1 h at room temperature. The catalysator was removed by Celite® 535 filtration then the solvent was removed. The residue was purified by flash chromatography (n-hexane:ethyl acetate 2:8 and 1% N,N-dimethylethanamin, Ø 2 cm, fraction size 10 mL, R_{f}=0.08). The titled compound was obtained as pale yellow oil.

### Example 28

### Preparation of 3-(Benzoylpiperidine-4-yl)2,3,4,5-tetrahydro-1H-3-benzazepine-1,7-diol

### step 28.1 Preparation of 3-(1-Benzoylpiperidine-4-yl)-7-benzyloxy-2,3,4,5-tetrahydro-1H-3-benzazepine-1-ol

To the secondary amine 7-Benzyloxy-2,3,4,5-tetrahydro-1H-3-benzazepine-1-ol (125.4 mg, 0.47 mmol), from step 11.4, and 1-benzoylpiperid-4-one (113.7 mg, 0.56 mmol) in CH₂Cl₂ (4 mL) are added NaBH(OAc)₃ (118.7 mg, 0.56 mmol). The reaction mixture was stirred for 16 h at room temperature and subsequently mixed with H₂O (10 mL). The aqueous phase was extracted three times with CH₂Cl₂ (10 mL) and the combined organic layers dried with Na₂SO₄. Then the solvent was removed and the residue was purified by flash chromatography (n-hexane:ethyl acetate 3:7 and 1% N,N-dimethylethanamine, Ø 2 cm, fraction size 10 mL, R_{f}=0.23). The titled compound was obtained as pale yellow resin.

### step 28.2 Preparation of 3-(Benzoylpiperidine-4-yl)2,3,4,5-tetrahydro-1H-3-benzazepine-1,7-diol

To 3-(1-Benzoylpiperidine-4-yl)-7-benzyloxy-2,3,4,5-tetrahydro-1H-3-benzazepine-1-ol (90.1 mg, 0.20 mmol), from step 28.1, in abs. CH₃OH (4 mL) was added Pd/C (60.0 mg, 10%). The suspension was stirred under H₂-atmosphere (1 bar) for 2 h at room temperature. The catalysator was removed by Celite® 535 filtration then the solvent was removed. The residue was purified by flash chromatography (ethyl acetate:CH₃OH 9:1 and 1% N,N-dimethylethanamine, ● 2 cm, fraction size 10 mL, R_{f}=0.26). The titled compound was obtained as colourless solid.

### Example 29

### 3-(1,4-Dioxaspiro[4.5]decane-8-yl)-2,3,4,5-tetrahydro-1H-3-benzazepine-1,7-diol

To 7-Benzyloxy-3-(1,4-dioxaspiro[4.5]decan-8-yl)-2,3,4,5-tetrahydro-1H-3-benzazepine-1-ol (89.3 mg, 0.22 mmol), from example 18, in abs. CH₃OH (6 mL) was added Pd/C (53.2 mg, 10%). The suspension was stirred under H₂-atmosphere (1 bar) for 1 h at room temperature. The catalysator was removed by Celite® 535 filtration then the solvent was removed. The residue was purified by flash chromatography (n-hexane:ethyl acetate 4:6 and 1% N,N-dimethylethanamin, Ø 2 cm, fraction size 10 mL, R_{f}=0.10). The titled compound was obtained as a colourless solid.

### Example 30

### Preparation of (1S, 2S)-7-Methoxy-2-methyl-3-(4-phenylbutyl)-2,3,4,5-tetrahydro-1H-3-benzazepine-1-ol and (1R, 2R)-7-Methoxy-2-methyl-3-(4-phenylbutyl)-2,3,4,5-tetrahydro-1H-3-benzazepine-1-ol

### step 30.1 Preparation of (S)-Alaninmethylester-Hydrochlorid

CH₃OH (20 mL) was cooled to −5° C. and SOCl₂ (4.35 mL, 0.06 mol) was added slowly. To the solution (S)-alanine (1.78 g, 0.02 mol) was added in several portions. After the (S)-alanine was dissolved the solution was allowed to come to room temperature and stirred for further 2 h. The excess of the SOCl₂ and the CH₃OH was removed by evaporation. The residue was dissolved in CH₃OH (30 mL) and the solvent was evaporated. This procedure was repeated twice. The titled compound was obtained as a colourless solid.

### step 30.2 Preparation of (S)-Methyl-2-(4-tosyl)aminopropionat

To (S)-alaninmethylester hydrochloride of step 30.1 (3.92 g, 28.1 mmol) in CH₂Cl₂ (95 mL) was added p-toluenesulfonic acid in several portions followed by the addition of triethyamine (7.45 mL, 70.3 mmol). The solution was stirred for 20 h at room temperature. The organic phase was mixed with 0.5M HCl (3×30 mL) and H₂O (3×30 mL). The organic layers were dried with Na₂SO₄ and concentrated in vacuum. The residue was purified by flash chromatography (n-hexane:ethyl acetate 7:3 and 1% N,N-dimethylethanamine, Ø 8 cm, fraction size 65 mL, R_{f}=0.62). The titled compound was obtained as colourless solid.

### step 30.3 Preparation of 2-(3-Methoxyphenyl)ethanol

3-Bromanisole (300 μL, 2.39 mmol) was dissolved under nitrogen atmosphere in abs. THF (35 mL) and cooled to −78° C. Afterwards n-butyllithium in n-hexane (1.48 M, 1.62 mL 2.39 mmol) was added dropwise during 30 min and subsequently stirred for 2 h at −78° C. In the following step ethylensulfat (355.3 mg, 2.87 mmol) was dissolved in abs. THF (5 mL) and transferred dropwise to the solution for 20 min. The reaction mixture was allowed to come to room temperature and stirred for 16 h. After the addition Of H₂O (10 mL) and conc. H₂SO₄ (1.8 mL) the suspension was heated at reflux for 47 h. Finally the solution was neutralized by 5M NaOH (20 mL) and extracted three times with CH₂Cl₂ (20 mL). The combined organic layers were dried with Na₂SO₄ and concentrated in vacuum. The residue was purified by flash chromatography (n-hexane:ethyl acetate 8:2, Ø 4 cm, fraction size 30 mL, R_{f}=0.16). The titled compound was obtained as colourless oil.

### step 30.4 Preparation of (S)-Methyl-2-{N-[2-(3-methoxyphenyl)ethyl]-N-(4-tosyl)amino}propionate

2-(3-Methoxyphenyl)ethanol (7.74 g, 0.05 mol), from step 30.3, dissolved in abs. THF (600 mL) was cooled to 0° C. under nitrogen atmosphere followed by the addition of (S)-Methyl-2-(4-tosyl)aminopropionat (13.1 g, 0.05 mol), from step 29.1, and Ph₃P (40.3 g, 0.15 mol). In the next step DIAD (29.7 mL, 0.15 mol) was added dropwise. After 1 h at 0° C. the reaction mixture was allowed to come to room temperature followed by stirring for 16 h. Then the solution was diluted with n-hexane (700 mL) and the precipitated Ph₃P═O was removed by filtration. The solvent was evaporated and the residue was purified by flash chromatography (n-hexane:ethyl acetate 8:2, Ø 8 cm, fraction size 65 mL, R_{f}=0.35). The titled compound was obtained as colourless oil.

### step 30.5 Preparation of (S)-2-{N-[2-(3-Methoxy-phenyl)ethyl]-N-(4-tosyl)amino}propion acid

(S)-Methyl-2-{N-[2-(3-methoxyphenyl)ethyl]-N-(4-tosyl)amino}propionate (1.54 g, 3.95 mmol), from step 30.4, was dissolved in THF:H₂O-Mixture (7:3, 60 mL) followed by the addition Of LiOH.H₂O (1.26 g, 19.8 mmol). The mixture war stirred for 24 h at room temperature. Afterwards the mixture was acidified with 6M HCl and the aqueous phase was extracted four times with diethyl ether (30 mL). The combined organic layers were dried with Na₂SO₄ and the solvent was evaporated. The residue was purified by flash chromatography (n-hexane:ethyl acetate 2:8, Ø 2 cm, fraction size 30 mL, R_{f}=0.35). The titled compound was obtained as colourless oil.

### step 30.6 Preparation of (S)-7-Methoxy-2-methyl-3-(4-tosyl)-2,3,4,5-tetrahydro-3-benzazepine-1-one in mixture with

### (S)-9-Methoxy-2-methyl-3-(4-tosyl)-2,3,4,5-tetrahydro-3-benzazepine-1-one

(S)-2-{N-[2-(3-Methoxy-phenyl)ethyl]-N-(4-tosyl)amino}propion acid (5.10 g, 14.0 mmol), from step 30.5, was dissolved in dichloromethane (300 mL) under nitrogen atmosphere and cooled to −30° C. Then trifluoracetic acid anhydride (9.56 mL, 68.0 mmol) was added and stirred for 1 h at −30° C. Afterwards SnCL₄ (6.55 mL, 56.0 mmol) was added slowly. After 22 h stirring at −30° C. the solution was neutralized with 5M NaOH at 0° C. and the aqueous phase was extracted three times with CH₂Cl₂ (50 mL). The combined organic layers were dried with Na₂SO₄ and the solvent was evaporated. The residue was purified by flash chromatography (n-hexane:ethyl acetate 7:3, Ø 8 cm, fraction size 65 mL, R_{f}=0.36).

The regioisomers (S)-7-Methoxy-2-methyl-3-(4-tosyl)-2,3,4,5-tetrahydro-3-benzazepine-1-one and (S)-9-Methoxy-2-methyl-3-(4-tosyl)-2,3,4,5-tetrahydro-3-benzazepine-1-one were obtained as colourless solid and were used in the following step as mixture.

### step 30. 7 Preparation of (1S,2S)-7-Methoxy-2-methyl-3-(4-tolyisulfony)-2,3,4,5-tetrahydro-1H-3-benzazepine-1-ol and

### (1R,26)-7-Methoxy-2-methyl-3-(4-tolyisulfony)-2,3,4,5-tetrahydro-1H-3-benzazepine-1-ol

A mixture of (S)-7-methoxy-2-methyl-3-(4-tosyl)-2,3,4,5-tetrahydro-3-benzazepine-1-one and compound (S)-9-Methoxy-2-methyl-3-(4-tosyl)-2,3,4,5-tetrahydro-3-benzazepine-1-one (6.34 g, 17.63 mmol) of step 30.6 were dissolved in THF (400 mL) under nitrogen atmosphere and cooled to −90° C. Afterwards a 2M solution of LiBH₄ (22.04 mL, 44.02 mmol) in THF was added slowly. After 24 h stirring at −90° C. H₂O (100 mL) was added and the reaction mixture was allowed to come to room temperature. The aqueous phase was extracted three times with CHCl₃ (40 mL) and the combined organic layers were dried with Na₂SO₄. The solvent was evaporated. The residue was purified by flash chromatography (n-hexane:ethyl acetate 7:3, Ø 8 cm, fraction size 65 mL, R_{f}((1S,2S)-7-Methoxy-2-methyl-3-(4-tolylsulfony)-2,3,4,5-tetrahydro-1H-3-benzazepine-1-ol)=0.45, R_{f}((1R,2S)-7-Methoxy-2-methyl-3-(4-tolylsulfony)-2,3,4,5-tetrahydro-1H-3-benzazepine-1-ol)=0.24). (1S,2S)-7-Methoxy-2-methyl-3-(4-tolylsulfony)-2,3,4,5-tetrahydro-1H-3-benzazepine-1-ol) was obtained as a colourless oil. (1R,2S)-7-Methoxy-2-methyl-3-(4-tolylsulfony)-2,3,4,5-tetrahydro-1H-3-benzazepine-1-ol was obtained as a colourless oil.

### step 30.8 Preparation of (1S,2S)-7-Methoxy-2-methyl-2,3,4,5-tetrahydro-1H-3-benzazepin-1-ol

To compound (1S,2S)-7-Methoxy-2-methyl-3-(4-tolylsulfony)-2,3,4,5-tetrahydro-1H-3-benzazepine-1-ol of step 30.7 (1.13 g, 3.26 mmol) dissolved in CH₃OH (170 mL) was added Mg (1.72 g, 71.7 mmol). The reaction mixture was heated at reflux for 18 h and afterwards acidified with 2M HCl and the unspent Mg was filtered. The solution was adjusted with 2M NaOH to an alkaline pH-value (pH 9-10), the water phase was subsequently extracted with CH₂Cl₂ (5×60 mL) and dried with Na₂SO₄. The solvent was evaporated and the residue was purified by flash chromatography (ethyl acetate:CH₃OH 9:1+2% N,N-dimethylethanamine, Ø 4 cm, fraction size 30 mL, R_{f}=0.08). The titled compound was obtained as a colourless solid.

step 30.9 Preparation of (1S, 2S)-7-Methoxy-2-methyl-3-(4-phenylbutyl)-2,3,4,5-tetrahydro-1H-3-benzazepine-1-ol and

### (1R, 2R)-7-Methoxy-2-methyl-3-(4-phenylbutyl)-2,3,4,5-tetrahydro-1H-3-benzazepine-1-ol

The compound preparation of (1S,2S)-7-Methoxy-2-methyl-2,3,4,5-tetrahydro-1H-3-benzazepin-1-ol of step 30.8 (70.7 mg, 0.34 mmol), 1-chlor-4-phenylbutane (108.5 μL, 0.68 mmol), TBAl (251.2 mg, 0.68 mmol) and K₂CO₃ (375.9 mg, 2.72 mmol) were dissolved in CH₃CN (9 mL). After 72 h heating at reflux the precipitation was filtered and the solvent was evaporated. The residue was purified by flash chromatography (n-hexane:ethyl acetate 7:3 and 1% N,N-Dimethylethanamin, Ø 2 cm, fraction size 10 mL, R_{f}=0.29). The titled enantiomers as a mixture were obtained as colourless solid.

The separation of the enantiomers was carried out on a chiral HPLC (column: Chiralpak® AD (Chiral Technologies Europe), solvent: n-Hexan:Isopropanol (95.5:4.5), t_{R} ((1S, 2S)-7-Methoxy-2-methyl-3-(4-phenylbutyl)-2,3,4,5-tetrahydro-1H-3-benzazepine-1-ol)=16.6 min, t_{R} (1R, 2R)-7-Methoxy-2-methyl-3-(4-phenylbutyl)-2,3,4,5-tetrahydro-1H-3-benzazepine-1-ol)=19.9 min).

(1S, 2S)-7-Methoxy-2-methyl-3-(4-phenylbutyl)-2,3,4, 5-tetrahydro-1H-3-benzazepine-1-ol was obtained as a colourless resin. (1R, 2R)-7-Methoxy-2-methyl-3-(4-phenylbutyl)-2,3,4,5-tetrahydro-1H-3-benzazepine-1-ol was obtained as a colourless solid.

### Example 31

### Preparation of (1R,2S)-2-Methyl-3-(4-phenylbutyl)-2,3,4,5-tetrahydro-1H-3-benzazepine-1,7-diol and (1S,2R)-2-Methyl-3-(4-phenylbutyl)-2,3,4,5-tetrahydro-1H-3-benzazepine-1,7-diol

### Step 31.1 Preparation of (S)-Alaninmethylester-hydrochloride

The procedure was carried out as described in step 30.1.

### Step 31.2 Preparation of (S)-Methyl-2-(4-tosyl)aminopropionate

The procedure was carried out as described in step 30.2.

### Step 31.3 Preparation of 2-(3-Benzyloxyphenyl)-ethanol

3-(2-Hydroxyethyl)phenole (216.4 mg, 1.6 mmol) and benzylbromide (203.8 pL, 1.7 mmol) were dissolved in a 10 mL microwave pressure vessel. To the solution K₂CO₃ (862.4 mg, 6.2 mmol) was added. Parameters of reaction in the microwave (Discover, CEM GmbH) were: standard mode, stir on, 220 watt power, maximum temperature 100° C., maximum pressure 4 bar, 5 min ramp time, 40 min hold time and 5 min cool off time. Afterwards the K₂CO₃ was filtered and the solvent was evaporated. The residue was purified by flash chromatography (n-hexane:ethyl acetate 8:2 , Ø 2 cm, fraction size 10 mL, R_{f}=0.15). The titled compound was obtained as a colourless solid.

### Step 31.4 Preparation of (S)-Methyl-2-{N-[2-(3-benzyloxyphenyl)ethyl]-N-(4-tosyl)amino}-propionate

2-(3-Benzyloxyphenyl)-ethanol of step 31.3 (7.74 g, 0.05 mol) was dissolved in abs. THF (600 mL) and cooled under nitrogen atmosphere to 0° C. To the solution (S)-methyl-2-(4-tosyl)aminopropionate of step 31.2 and Ph₃P (40.3 g, 0.15 mol) were added and afterwards DIAD (29.7 mL, 0.15 mol) was added drop wise. After 1 h at 0° C. the reaction mixture was allowed to come to room temperature and stirred for 16 h. To the solution n-hexane (700 mL) was added and the precipitated Ph₃P═O was removed by filtration. The solvent was evaporated and residue was purified by flash chromatography (n-hexane:ethyl acetate 8:2, Ø 8 cm, fraction size 65 mL, R_{f}=0.35). The titled compound was obtained as colourless oil.

### Step 31.5 Preparation of (S)-2-{N-[2-(3-Benzyloxyphenyl)ethyl]-N-(4-tosyl)amino}propionic acid

To (S)-methyl-2-{N-[2-(3-benzyloxyphenyl)ethyl]-N-(4-tosyl)amino}propionate of step 31.4 (785.4 mg, 1.7 mmol) dissolved in THF (21 mL) was added H₂O (9 mL) and LiOH.H₂O (660 mg, 15,1 mmol). The mixture was stirred at room temperature for 23 h, afterwards the pH was adjusted to pH=6 and extracted with CH₂Cl₂ (3×15 mL). The combined organic layers were dried with K₂CO₃ and the solvent was evaporated. The residue was purified by flash chromatography (n-hexane:ethyl acetate 2:8, Ø 3 cm, fraction size 30 mL, R_{f}=0.43). The titled compound was obtained as colourless oil.

### Step 31.6 Preparation of (S)-7-Benzyloxy-2-methyl-3-(4-tosyl)-2,3,4,5-tetrahydro-3-benzazepine-1-one in mixture with

### (S)-9-Benzyloxy-2-methyl-3-(4-tosyl)-2,3,4,5-tetrahydro-3-benzazepine-1-one

To (S)-2-{N-[2-(3-benzyloxyphenypethyl]-N-(4-tosyl)amino}propionic acid of step 31.5 (4.77 g, 10.5 mmol) wad added CH₂Cl₂ (295 mL) under nitrogen atmosphere at −15° C. After addition of trifluoracetic acid anhydride (5.56 mL, 39.4 mmol) the mixture was stirred for 30 min. Then SnCl₄ (4.61 mL, 39.4 mmol) was added drop wise to the mixture and subsequently stirred for 24h at −15° C. H₂O (150 mL) was added drop wise and the mixture was allowed to come to room temperature. The water phase was neutralized with 2M NaOH, saturated with brine and extracted with CH₂Cl₂ (3×75 mL). The combined organic layers were dried with Na₂SO₄ and the solvent was evaporated. The residue was dissolved in acetone (180 mL) followed by the addition of K₂CO₃ (5.44 g, 39.4 mmol) and benzylbromide (1.56 mL, 13.1 mmol). The suspension was heated at reflux for 6 h. The insoluble residue was filtered and the solvent was evaporated. The residue was purified by flash chromatography (n-hexane:ethyl acetate 7:3, Ø 8 cm, fraction size 65 mL, R_{f} ((S)-7-Benzyloxy-2-methyl-3-(4-tosyl)-2,3,4,5-tetrahydro-3-benzazepine-1-one (30a) and (S)-9-Benzyloxy-2-methyl-3-(4-tosyl)-2,3,4,5-tetrahydro-3-benzazepine-1-one)=0.34).

The mixture of the titled compounds was obtained as a colourless solid.

The regioisomers (S)-7-Benzyloxy-2-methyl-3-(4-tosyl)-2,3,4,5-tetrahydro-3-benzazepine-1-one (30a) and (S)-9-Benzyloxy-2-methyl-3-(4-tosyl)-2,3,4,5-tetrahydro-3-benzazepine-1-one could not be separated by flash chromatography and were further used as mixture.

### Step 31.7 Preparation of (1R,2S)-7-Benzyloxy-2-methyl-3-(4-tosyl)-2,3,4,5-tetrahydro-1H-3-benzazepine-1-ol and

### (1S,2S)-7-Benzyloxy-2-methyl-3-(4-tosyl)-2,3,4,5-tetrahydro-1H-3-benzazepine-1-ol

To a mixture of the compounds (S)-7-Benzyloxy-2-methyl-3-(4-tosyl)-2,3,4,5-tetrahydro-3-benzazepine-1-one (30a) and (S)-9-Benzyloxy-2-methyl-3-(4-tosyl)-2,3,4,5-tetrahydro-3-benzazepine-1-one of step 31.6 (3.11 g, 7.16 mmol) were added abs. CH₃OH (75 mL) in nitrogen atmosphere. The suspension was added NaBH₄ (0.55 g, 14.3 mmol) in several portions and stirred for 20 h at room temperature. After the addition of water H₂O (30 mL) the water phase was extracted with CH₂Cl₂ (3×20 mL) and the combined organic layers were dried with Na₂SO₄.

The solvent was evaporated and the residue was purified by flash chromatography (n-hexane:ethyl acetate 7:3, Ø 8 cm, fraction size 65 mL, R_{f} ((1R,2S)-7-Benzyloxy-2-methyl-3-(4-tosyl)-2,3,4,5-tetrahydro-1H-3-benzazepine-1-ol)=0.44, R_{f} ((1S,2S)-7-Benzyloxy-2-methyl-3-(4-tosyl)-2,3,4,5-tetrahydro-1H-3-benzazepine-1-ol)=0.29).

The compound (1S,2S)-7-Benzyloxy-2-methyl-3-(4-tosyl)-2,3,4,5-tetrahydro-1H-3-benzazepine-1-ol was obtained as a colourless solid. The compound (1R,2S)-7-Benzyloxy-2-methyl-3-(4-tosyl)-2,3,4,5-tetrahydro-1H-3-benzazepine-1-ol was obtained as a colourless solid.

### Step 31.8 Preparation of (1R,2S)-7-Benzyloxy-2-methyl-2,3,4,5-tetrahydro-1H-3-benzazepine-1-ol

To compound (1R,2S)-7-Benzyloxy-2-methyl-3-(4-tosyl)-2,3,4,5-tetrahydro-1H-3-benzazepine-1-ol of step 31.7 (1.07 g, 2.45 mmol) in CH₃OH (125 mL) was added Mg (1.8 g, 75.0 mmol). The reaction mixture was heated at reflux for 36 h. Afterwards the pH was adjusted with 2M HCl to pH=6 and unspent Mg was filtered, followed by the addition of NaOH to adjust to an alkaline pH-value (pH 10) of the filtrate. The water phase was subsequently extracted with CH₂Cl₂ (5×50 mL). The combined organic layers were dried with Na₂SO₄ and the solvent was evaporated. The residue was purified by flash chromatography (ethyl acetate:CH₃OH 9:1+2% N,N-dimethylethanamine, Ø 6 cm, fraction size 65 mL, R_{f}=0.13) and finally purified in CH₃CN by fractionated crystallization. The titled compound was obtained as a colourless solid.

### Step 31.9 Preparation of (1R,2S)-7-Benzyloxy-2-methyl-3-(4-phenylbutyl)-2,3,4,5-tetrahydro-1H-3-benzazepine-1-ol and

### (1S,2R)-7-Benzyloxy-2-methyl-3-(4-phenylbutyl)-2,3,4,5-tetrahydro-1H-3-benzazepine-1-ol

To (1R,2S)-7-Benzyloxy-2-methyl-2,3,4,5-tetrahydro-1H-3-benzazepine-1-ol of step 31.8 (101.2 mg, 0.36 mmol), 1-chlor-4-phenylbutane (88.8 μL, 0.54 mmol), Tetrabutylammonium iodide (TBAl, 199.5 mg, 0.54 mmol) and K₂CO₃ (248.8 mg, 1.80 mmol) was added CH₃CN (14 mL) and subsequently heated at reflux for 72 h. The precipitation was filtered and the solvent was evaporated. The residue was purified by flash chromatography (n-hexane:ethyl acetate 9:1+1% N-N-dimethylethanamine, Ø 3 cm, fraction size 10 mL, R_{f}=0.30). The mixture of the titled compounds was obtained as a colourless solid.

The separation of the enantiomers (1R,2S)-7-Benzyloxy-2-methyl-3-(4-phenylbutyl)-2,3,4,5-tetrahydro-1H-3-benzazepine-1-ol and (1S,2R)-7-Benzyloxy-2-methyl-3-(4-phenylbutyl)-2,3,4,5-tetrahydro-1H-3-benzazepine-1-ol was carried out on a chiral HPLC according to (column: Chiralpak® AD, n-hexane:isopropanol 75:25, t_{R}=15.8 min, t_{R}=10.2 min)

The compound (1R,2S)-7-Benzyloxy-2-methyl-3-(4-phenylbutyl)-2,3,4,5-tetrahydro-1H-3-benzazepine-1-ol was obtained as a colourless oil.

The compound (1S,2R)-7-Benzyloxy-2-methyl-3-(4-phenylbutyl)-2,3,4,5-tetrahydro-1H-3-benzazepine-1-ol was obtained as a colourless oil.

Step 31.10 Preparation of (1R,2S)-2-Methyl-3-(4-phenylbutyl)-2,3,4,5-tetrahydro-1H-3-benzazepine-1,7-diol in mixture with

### (1S,2R)-2-Methyl-3-(4-phenylbutyl)-2,3,4,5-tetrahydro-1H-3-benzazepine-1,7-diol

To the mixture of the compounds (1R,2S)-7-Benzyloxy-2-methyl-3-(4-phenylbutyl)-2,3,4,5-tetrahydro-1H-3-benzazepine-1-ol and (1S,2R)-7-Benzyloxy-2-methyl-3-(4-phenylbutyl)-2,3,4,5-tetrahydro-1H-3-benzazepine-1-ol of step 31.9 (163.2 mg, 0.39 mmol) dissolved in abs. CH₃OH (16 mL) was added Pd/C (110.2 mg, 10%). The suspension was stirred 1 h under H₂-atmosphere (1 bar) at room temperature. The catalysator was removed by Celite® 535 filtration then the solvent was removed. The residue was purified by flash chromatography (n-hexane:ethyl acetate 5:5+1% N,N-dimethylethanamine, Ø 2 cm, fraction size 10 mL, R_{f}=0.36). The mixture of the titled compounds was obtained as a pale yellow solid.

The separation of the enantiomers (1R,2S)-2-Methyl-3-(4-phenylbutyl)-2,3,4,5-tetrahydro-1H-3-benzazepine-1,7-diol and (1S,2R)-2-Methyl-3-(4-phenylbutyl)-2,3,4,5-tetrahydro-1H-3-benzazepine-1,7-diol was carried out on a chiral preparative HPLC (n-hexane:ethanol (90:10), t_{R} ((1R,2S)-2-Methyl-3-(4-phenylbutyl)-2,3,4,5-tetrahydro-1H-3-benzazepine-1,7-diol)=69.7 min, t_{R} ((1S,2R)-2-Methyl-3-(4-phenylbutyl)-2,3,4,5-tetrahydro-1H-3-benzazepine-1,7-diol)=77.9 min).

The compound (1R,2S)-2-Methyl-3-(4-phenylbutyl)-2,3,4,5-tetrahydro-1H-3-benzazepine-1,7-diol was obtained as a pale yellow resin.

The compound (1S,2R)-2-Methyl-3-(4-phenylbutyl)-2,3,4,5-tetrahydro-1H-3-benzazepine-1,7-diol was obtained as a pale yellow oil.

### Example 32

### Preparation of (1S,2S)-2-Methyl-3-(4-phenylbutyl)-2,3,4,5-tetrahydro-1H-3-benzazepine-1,7-diol

### Step 32.1 Preparation of (R)-Alaninmethylester-hydrochloride

The procedure was carried out as described for (S)-alaninmethylester-hydrochloride in step 31.1 in CH₃OH (112 mL), SOCl₂ (23.9 mL, 0.33 mol) and (R)-alanine (10.0 g, 0.11 mol). The titled compound was obtained as a colourless solid.

### Step 32.2 Preparation of (R)-Methyl-2-(4-tosyl)aminopropionate

The procedure was carried out as described for (S)-methyl-2-(4-tosyl)aminopropionate in step 31.2 with (R)-alaninmethylester-hydrochloride (9.64 g, 69.4 mmol), CH₂Cl₂ (160 mL), p-toluolsulfonylchloride (19.8 g, 97.0 mmol) and triethylamine (15.45 mL, 0.15 mol).

### Step 32.3 Preparation of 2-(3-Benzyloxyphenyl)-ethanol

The procedure was carried out as described in step 31.3.

### Step 32.4 Preparation of (R)-Methyl-2-{N-[2-(3-benzyloxyphenyl)ethyl]-N-(4-tosyl)amino}-propionate

The procedure was carried out as described above in step 31.4 with (R)-Methyl-2-(4-tosyl)aminopropionate (3.83 g, 14.9 mmol), Ph₃P (10.4 g, 39.6 mmol), DIAD (7.68 mL, 39.6 mmol) and abs. THF (240 mL). The titled compound was obtained as colourless oil.

### Step 32.5 Preparation of (R)-2-{N-[2-(3-Benzyloxyphenyl)ethyl]-N-(4-tosyl)amino}propionic acid

The procedure was carried out as described above in step 31.5 with (R)-methyl-2-{N-[2-(3-benzyloxyphenyl)ethyl]-N-(4-tosyl)amino}propionate (4.40 g, 9.42 mmol), LiOH.H₂O (3.38 g, 80.4 mmol) and THF (130 mL). The titled compound was obtained as colourless oil.

### Step 32.6 Preparation of (R)-7-Benzyloxy-2-methyl-3-(4-tosyl)-2,3,4,5-tetrahydro-3-benzazepine-1-one

in mixture with

### (R)-9-Benzyloxy-2-methyl-3-(4-tosyl)-2,3,4,5-tetrahydro-3-benzazepine-1-one

The procedure was carried out as described above in step 31.6 with (R)-2-{N-[2-(3-Benzyloxyphenypethyl]-N-(4-tosyl)amino}propionic acid (3.95 g, 8.72 mmol), CH₂Cl₂ (250 mL), trifluoracetic acid anhydride (3.69 mL, 26.2 mmol) and SnCl₄ (3.06 mL, 26.16 mmol). The mixture of the titled compounds was obtained as a pale yellow solid. The R_{f} of the regioisomers was 0.34. The compounds were further used as mixture.

### Step 32.7 Preparation of (1S,2R)-7-Benzyloxy-2-methyl-3-(4-tosyl)-2,3,4,5-tetrahydro-1H-3-benzazepine-1-ol and

### (1R,2R)-7-Benzyloxy-2-methyl-3-(4-tosyl)-2,3,4,5-tetrahydro-1H-3-benzazepine-1-ol

The procedure was carried out as described above in step 31.6 with the mixture of (R)-7-Benzyloxy-2-methyl-3-(4-tosyl)-2,3,4,5-tertrahydro-3-benzazepine-1-one and (R)-9-Benzyloxy-2-methyl-3-(4-tosyl)-2,3,4,5-tetrahydro-3-benzazepine-1-one (5.20 g, 12.0 mmol), NaBH₄ (909.2 mg, 23.9 mmol) and CH₃OH (125 mL).

The compound (1S,2R)-7-Benzyloxy-2-methyl-3-(4-tosyl)-2,3,4,5-tetrahydro-1H-3-benzazepine-1-ol was obtained as a colourless solid.

The compound (1R,2R)-7-Benzyloxy-2-methyl-3-(4-tosyl)-2,3,4,5-tetrahydro-1H-3-benzazepine-1-ol was obtained as a colourless solid.

### Step 32.8 Preparation of (1S,2S)-7-Benzyloxy-2-methyl-2,3,4,5-tetrahydro-1H-3-benzazepine-1-ol in mixture with

### (1R,2R)-7-Benzyloxy-2-methyl-2,3,4,5-tetrahydro-1H-3-benzazepine-1-ol

To the compounds (1R,2R)-7-Benzyloxy-2-methyl-3-(4-tosyl)-2,3,4,5-tetrahydro-1H-3-benzazepine-1-ol of step 32.7 and (1S,2S)-7-Benzyloxy-2-methyl-3-(4-tosyl)-2,3,4,5-tetrahydro-1H-3-benzazepine-1-ol of step 32.7 and step 31.7 (1.13 g, 2.58 mmol) dissolved in CH₃OH (130 mL) was added Mg (1.37 g, 56.9 mmol). The reaction mixture was heated at reflux for 36 h. Afterwards the pH was adjusted with 2M HCl to pH=3 and unspent Mg was filtered, followed by the addition of NaOH to adjust to an alkaline pH-value (pH 10) of the filtrate. The water phase was subsequently extracted with CH₂Cl₂ (5×50 mL). The combined organic layers were dried with Na₂SO₄ and the solvent was evaporated.

The residue was purified by flash chromatography (ethyl acetate:CH₃OH, 9.5:0.5+2% N,N-dimethylethanamine, Ø 6 cm, fraction size 30 mL, R_{f}=0.23). The mixture of the titled compounds was obtained as a pale yellow solid.

### Step 32.9 Preparation of (1S,2S)-7-Benzyloxy-2-methyl-3-(4-phenyibutyl)-2,3,4,5-tetrahydro-1H-3-benzazepine-1-ol and

### (1R,2R)-7-Benzyloxy-2-methyl-3-(4-phenyibutyl)-2,3,4,5-tetrahydro-1H-3-benzazepine-1-ol

The mixture of compound (1S,2S)-7-Benzyloxy-2-methyl-2,3,4,5-tetrahydro-1H-3-benzazepine-1-ol and (1R,2R)-7-Benzyloxy-2-methyl-2,3,4,5-tetrahydro-1H-3-benzazepine-1-ol of step 32.8 (122.2 mg, 0.43 mmol) was added 1-chlor-4-phenylbutane (108.0 μL, 0.66 mmol), TBAl (241.0 mg, 0.65 mmol) and K₂CO₃ (301.0 mg, 2.18 mmol) in CH₃CN (20 mL) and subsequently heated at reflux for 72 h. The precipitation was filtered and the solvent was evaporated. The residue was purified by flash chromatography (n-hexane:ethyl acetate 7:3+1% N,N-dimethylethanamine, Ø 3 cm, fraction size 10 mL, R_{f}=0.22). The mixture of the titled compounds was obtained as a colourless solid.

The separation of the enantiomers compound (1S,2S)-7-Benzyloxy-2-methyl-3-(4-phenylbutyl)-2,3,4,5-tetrahydro-1H-3-benzazepine-1-ol and (1R,2R)-7-Benzyloxy-2-methyl-3-(4-phenylbutyl)-2,3,4,5-tetrahydro-1H-3-benzazepine-1-ol was carried out on a chiral preparative HPLC (n-hexane:ethanol:isopropanol 96:3:1, flow rate 6.9 mL/min, t_{R} ((1S,2S)-7-Benzyloxy-2-methyl-3-(4-phenylbutyl)-2,3,4,5-tetrahydro-1H-3-benzazepine-1-ol)=48.6 min, t_{R} ((1R,2R)-7-Benzyloxy-2-methyl-3-(4-phenylbutyl)-2,3,4,5-tetrahydro-1H-3-benzazepine-1-ol)=58.6 min).

The titled compound (1S,2S)-7-Benzyloxy-2-methyl-3-(4-phenylbutyl)-2,3,4,5-tetrahydro-1H-3-benzazepine-1-ol was obtained as colourless resin.

The titled compound (1R,2R)-7-Benzyloxy-2-methyl-3-(4-phenylbutyl)-2,3,4,5-tetrahydro-1H-3-benzazepine-1-ol was obtained as colourless resin.

### Step 32.10 Preparation of (1S,25)-2-Methyl-3-(4-phenyibutyl)-2,3,4,5-tetrahydro-1H-3-benzazepine-1,7-diol

To compound (1S,2S)-7-Benzyloxy-2-methyl-3-(4-phenylbutyl)-2,3,4,5-tetrahydro-1H-3-benzazepine-1-ol (60.1 mg, 0.14 mmol) dissolved in abs. CH₃OH (6 mL) was added Pd/C (50.0 mg, 10%) The suspension was stirred 1 h under H₂-atmosphere (1 bar) at room temperature. The catalysator was removed by Celite® 535 filtration then the solvent was removed. The residue was purified by flash chromatography (n-hexane:ethyl acetate: 5+1% N,N-dimethylethanamine, Ø 2 cm, fraction size 10 mL, R_{f}=0.13). The titled compound was obtained as a colourless solid.

### Example 33

### (1R,2R)-2-Methyl-3-(4-phenylbutyl)-2,3,4,5-tetrahydro-1H-3-benzazepine-1,7-diol

The procedure was carried out as described above in Step 32.10 with compound (1R,2R)-7-Benzyloxy-2-methyl-3-(4-phenylbutyl)-2,3,4,5-tetrahydro-1H-3-benzazepine-1-ol (73.6 mg, 0.18 mmol), CH₃OH (6 mL) and Pd/C (50.2 mg, 10%). The titled compound was obtained as a colourless solid.

### Example 34

### Determination of the Affinity to the Polyamine Binding Site of the NR2B Subunit of the NMDA Receptor

The determination was performed using a filtration-based receptor binding assay on 96-well-multiplates for the polyamine binding site of the NR2B subunit of the NMDA receptor using [³H]-Ifenprodil as radioligand.

### Cell Culture and Preparation of Membrane Homogenates

Mouse L(tk-) cells stably transfected with the dexamethasone-inducible eukaryotic expression vectors pMSG NR1-1a, pMSG NR2B in a 1:5 ratio were a generous gift from D. 1835 Steinhilber (Department of Pharmacy, University of Frankfurt, Germany). The transformed L(tk-) cells were grown in Modified Earl's Medium (MEM) containing 10% of standardized FCS (Biochrom AG, Berlin, Germany). The expression of the NMDA receptor at the cell surface was induced after the cell density of the adherent growing cells had reached approximately 90% of confluency. For the induction, the original growth medium was replaced by growth medium containing 4 μM dexamethasone and 4 μM ketamine (final concentration). After 24 h the cells were harvested by trypsination and pelleted (10 min, 5,000×g, Hettich Rotina 35R centrifuge, Tuttlingen, Germany).

For the binding assay, the cell pellet was resuspended in PBS buffer and the number of cells was determined using an improved Neubauer's counting chamber (VWR, Darmstadt, Germany). Subsequently, the cells were lysed by sonication (4° C., 6×10 s cycles with breaks of 10 sec). The resulting cell fragments were centrifuged with a high performance cool centrifuge (20,000×g, 4° C., Sorvall RC-5 plus, Thermo Scientific). The supernatant was discarded and the pellet resuspended in a defined volume of phosphate buffer saline (PBS) yielding cell fragments of approximately 500,000 cells/mL. The suspension of membrane homogenates was sonicated again (4° C., 2×10 s cycles with a break of 10 min) and stored at −80° C.

### Receptor Binding Assay

The competitive binding assay was performed with the radioligand [³H]-Ifenprodil (60 Ci/mmol; Perkin Elmer) using standard 96-well-multiplates (Diagonal, Muenster, Germany). The thawed cell membrane preparation (about 20 μg protein) was incubated with 6 different concentrations of test compounds usually 10 μM, 1 μM, 100 nM, 10 nM, 1 nM and 0.1 nM, 5 nM [³H]-Ifenprodil, and TRIS/EDTA-buffer (5 mM/1 mM, pH 7.5) in a total volume of 200 μL for 180 min at 37° C. The cell membrane suspension was added last. All experiments were carried out in triplicates. The incubation was terminated by rapid filtration through filtermats using a cell harvester (MicroBeta FilterMate-96 Harvester, Perkin Elmer). Prior to harvesting, the filtermats were presoaked in 0.5% aqueous polyethylenimine for 2 h at room temperature. After washing each well five times with 300 μL of water, the filtermats were dried at 95° C. Subsequently, the solid scintillator was placed on the filtermat and melted at 95° C. After 5 min, the solid scintillator was allowed to solidify at room temperature. The bound radioactivity trapped on the filters was counted in the scintillation analyzer (Microbeta Counter, Perkin Elmer). The overall counting efficiency was 20%. The non-specific binding was determined with 10 μM unlabeled Ifenprodil.

### Protein Determination

The protein concentration was determined by the method of Bradford, modified by Stoscheck. The Bradford solution was prepared by dissolving 5 mg of Coomassie Brilliant Blue G 250 in 2.5 mL EtOH (95%, v/v). 10 mL deionized H₂O and 5 mL phosphoric acid (85%, m/v) were added to this solution, the mixture was stirred and filled up to a total volume of 50.0 mL with deionized water. The calibration was carried out using bovine serum protein as a standard in concentrations between 0.1 and 10 mg /L. In a 96-well standard multiplate, 10 μL of the calibration solution or 10 μL of the membrane receptor preparation were mixed with 190 μL of the Bradford solution, respectively. After 5 min, the UV absorption of the protein-dye complex at λ=595 nm was measured with a platereader (Tecan Genios, Tecan, Crailsheim, Germany).

### Saturation Experiment

The saturation analysis was performed by incubating increasing concentrations of [³H]-Ifenprodil (0.5 nM, 1 nM, 2.5 nM, 5 nM, 10 nM, 15 nM, 20 nM, 25 nM and 50 nM) together with 20 μg of the receptor protein in TRIS/EDTA-buffer (5 mM/1 mM, pH 7.5) for 2 h at 37° C. For each concentration, the nonspecific binding was determined with an excess of non-labelled Ifenprodil (10 μM). K_{d} and Bₘₐₓ were calculated as described in the “Data analysis” section.

### Data Analysis

Data analysis was performed with Graph Pad Prism® Software, Version 3.0 (Graph Pad Software Inc., San Diego, Calif., USA).

Saturation analyses were made by nonlinear regression using the “one-site-saturation” calculation method. The IC₅₀ values of the test compounds used in the competitive binding experiments were determined by nonlinear regression using the “one-site-competition” calculation method. Subsequently, the K, values of the test compounds were calculated according to the equation of Cheng and Prusoff (Y. Cheng, H. W. Prusoff, Biochem. Pharmacol., 1973, 22, 3099-3108). The K, values are given as mean values from three independent experiments±Standard Error of the Mean (S.E.M).

The determination of the affinity to the polyamine binding site of the NR2B subunit of the NMDA receptor was performed for the compounds according to formulas (1) to (30).

It was observed that most of the compounds according to formulas (1) to (30) showed an affinity to the polyamine binding site of the NR2B subunit of the NMDA receptor in the nanomolecular range. The compounds according to formulas (1) to (30) showed an affinity to the NR2B binding site of the NMDA receptor of 5.12 nM, 591±150 nM, 192±26 nM, 15.3±1.2 nM, 325 nM, 258 nM, 1.04 pM, 1.88 pM, 249±63 nM, 45.2 nM, 107 nM, 46 nM, 99 nM, 60.4 nM, 1.24 μM, 7.68 μM, 209 nM, 586 nM, 156 nM, 2.51 pM, 4.86 nM, 23.4 nM, 34.2 nM, 47.3 nM, 1.12 nM, 393 nM, 12.3 nM, 22.1 nM, 171 nM, and 16.4 nM, respectively.

This shows that the compounds according to formulas (1) to (30) exhibit a high affinity to the polyamine binding site of the NR2B subunit.

### Example 35

### Determination of the Affinity to the Phencyclidine Binding Site of the NMDA Receptor

### Materials and General Procedures

The pig brains were a donation of the local slaughterhouse (Coesfeld, Germany). Homogenizer: Elvehjem Potter (B. Braun Biotech International, Melsungen, Germany). Centrifuge: High-speed cooling centrifuge model Sorvall RC-5C plus (Thermo Fisher Scientific, Langenselbold, Germany). Filter: Printed Filtermat Typ A and B (Perkin Eimer LAS, Rodgau-Jugesheim, Germany), presoaked in 0.5% aqueous polyethylenimine for 2 h at room temperature before use. TRIS/EDTA-buffer was prepared by dissolving 606 mg Tris-Base and 372 mg Na-EDTA (both from Sigma-Aldrich, Deisenhofen, Germany) in 900 mL H₂O. Before filling to the final volume of 1000 mL, the pH of the solution was adjusted to pH=7.5 by dropwise addition of 1M HC1. The filtration was carried out with a MicroBeta FilterMate-96 Harvester (Perkin Eimer). The scintillation analysis was performed using Meltilex (Typ A or B) solid scintillator (Perkin Eimer). The solid scintillator was melted on the filtermat at a temperature of 95° C. for 5 minutes. After solidifying of the scintillator at room temperature, the scintillation was measured using a MicroBeta Trilux scintillation analyzer (Perkin Elmer). The overall counting efficiency was 20%. All experiments were carried out in triplicates using standard 96-well-multiplates (Diagonal, Muenster, Germany). The IC₅₀-values were determined in competition experiments with at least six concentrations of the test compounds, usually 10 μM, 1 μM, 100 nM, 10 nM, 1 nM and 0.1 nM, and were calculated with the program GraphPad Prism® 3.0 (GraphPad Software, San Diego, Calif., USA) by non-linear regression analysis. The Kᵢ values were calculated according to the formula of Cheng and Prusoff (Y. Cheng, H. W. Prusoff, Biochem. Pharmacol., 1973, 22, 3099-3108). The K,values are given as mean value+SEM from three independent experiments.

### Preparation of the Tissue

Fresh pig brain cortex was homogenized with the potter (500-800 rpm, 10 up-and-down strokes) in 6 volumes of cold 0.32 M sucrose. The suspension was centrifuged at 1200×g for 10 min at 4° C. The supernatant was separated and centrifuged at 23500×g for 20 min at 4° C. The pellet was resuspended in 5-6 volumes of TRIS/EDTA-buffer (5 mM/1 mM, pH 7.5) and centrifuged again at 31000×g (20 min, 4° C.). This procedure was repeated twice. The final pellet was resuspended in 5-6 volumes of buffer, the protein concentration was determined according to the method of Bradford (M. M. Bradford, Anal. Biochem., 1976, 72, 248-254) using bovine serum albumin as standard, and subsequently the preparation was frozen (−80° C.) in 1.5 mL portions containing about 0.8 mg protein/mL.

### Performance of the Assay

The test was performed with the radioligand [³H]-(+)-MK-801 (22.0 Ci/mmol; Perkin Elmer). The thawed membrane preparation (about 100 pg of the Protein) was incubated with various concentrations of test compounds, 2 nM [³H]-(+)-MK-801, and TRIS/EDTA-buffer (5 mM/1 mM, pH 7.5) in a total volume of 200 μL for 150 min at room temperature. The incubation was terminated by rapid filtration through the presoaked filtermats using a cell harvester. After washing each well five times with 300 μL of water, the filtermats were dried at 95° C. The bound radioactivity trapped on the filters was counted in the scintillation analyzer as described in the “General procedures” section. The non-specific binding was determined with 10 μM unlabeled (+)-MK-801. The Kd-value of (+)-MK-801 is 2.26 nM (T. Utech, Dissertation, 2003, Universität Freiburg).

The determination of the affinity to the phencyclidine binding site of the NMDA receptor was performed for the compounds according to formulas (1) to (30).

It was observed that the compounds according to formulas (1) to (30) showed only minor affinity to the phencyclidine binding site of the NMDA receptor, wherein inhibition of the phencyclidine binding site only was detected at concentrations above 1 μM or 10 μM.

This shows that the compounds according to formulas (1) to (30) exhibit a good selectivity to the NR2B subunit of the NMDA receptor in comparison to the phencyclidine binding site of the NMDA receptor.

### Example 36

### Determination of the Affinity to the σ₁ Receptor

Unless defined otherwise, materials and general procedures were as described according to example 35.

### Preparation of the Tissue

Five guinea pig brains (Harlan Winkelmann, Borchen, Germany) were homogenized with the potter (500-800 rpm, 10 up-and-down strokes) in 6 volumes of cold 0.32 M sucrose. The suspension was centrifuged at 1200×g for 10 min at 4° C. The supernatant was separated and centrifuged at 23500×g for 20 min at 4° C. The pellet was resuspended in 5-6 volumes of Buffer (50 mM TRIS, pH 7.4) and centrifuged again at 23500×g (20 min, 4° C.). This procedure was repeated twice. The final pellet was resuspended in 5 to 6 volumes of Buffer, the protein concentration was determined according to the method of Bradford using bovine serum albumin as standard, and subsequently the preparation was frozen (−80° C.) in 1.5 mL portions containing about 1.5 mg protein/mL.

### Performance of the Assay

The test was performed with the radioligand [³H]-(+)-pentazocine (42.5 Ci/mmol; Perkin Elmer). The thawed membrane preparation (about 75 μg of the protein) was incubated with various concentrations of test compounds, 2 nM [³H]-(+)-pentazocine, and Buffer (50 mM TRIS, pH 7.4) in a total volume of 200 μL for 180 min at 37° C. The incubation was terminated by rapid filtration through the presoaked filtermats using a cell harvester. After washing each well five times with 300 μl of water, the filtermats were dried at 95° C. The bound radioactivity trapped on the filters was counted in the scintillation analyzer as described in the “General procedures” section. The non-specific binding was determined with 10 μM unlabeled (+)-pentazocine. The Kd-value of (+)-pentazocine is 2.9 nM.

The determination of the affinity to the σ₁ receptor was performed for the compounds according to formulas (1) to (30).

It was observed that the compounds according to formulas (1) to (5), (7) to (14), (16), (17), (18), (20) to (25), and (27) to (30) showed an affinity to the σ₁ receptor of 182±67 nM, 44.6±21.4 nM, 33.1±29.8 nM, 194 nM, 65.3±12.3 nM, 94.7±51 nM, 155 nM, 293±101 nM, 349 nM, 45.6 nM, 376 nM, 604 nM, 44.3±14.1 nM, 172 nM, 42.1±9.9 nM, 3.21±1.42 nM, 10.4±1.4 nM, 123±19 nM, 82.2 nM, 200±62 nM, 1.05 μM, 183 nM, 85.5 nM, 393 nM, 40.8 nM, and 846 nM, respectively, wherein the compounds according to formulas (6), (15), (19), and (26) showed inhibition of the the compounds according to formulas (6), (15), (19), and (26) showed inhibition of the aₗ receptor only at concentrations above 1 μM or 10 μM. σ₁ receptor only at concentrations above 1 μM or 10 μM.

This shows that the compounds according to formulas (1) to (5), (7) to (14), (16), (17), (18), (20) to (25), and (27) to (30) showed a good affinity to the σ₁ receptor, too.

### Example 37

### Determination of Affinity to the σ₂ receptor

Unless defined otherwise, materials and general procedures were as described according to example 35.

### Preparation of the Tissue

Two rat livers (Harlan Winkelmann, Borchen, Germany) were cut into smaller pieces and homogenized with the potter (500-800 rpm, 10 up-and-down strokes) in 6 volumes of cold 0.32 M sucrose. The suspension was centrifuged at 1200×g for 10 min at 4° C. The supernatant was separated and centrifuged at 31000×g for 20 min at 4° C. The pellet was resuspended in 5-6 volumes of buffer (50 mM TRIS, pH 8.0) and incubated at room temperature for 30 minutes. After the incubation, the suspension was centrifuged again at 31000×g for 20 min at 4° C. The final pellet was resuspended in 5 to 6 volumes of buffer, the protein concentration was determined according to the method of Bradford using bovine serum albumin as standard, and subsequently the preparation was frozen (−80° C.) in 1.5 mL portions containing about 2 mg protein/mL.

### Performance of the Assay

The test was performed with the radioligand [³H]-ditolylguanidine (50 Ci/mmol; ARC). The thawed membrane preparation (about 100 lig of the protein) was incubated with various concentrations of test compounds, 3 nM [³H]-ditolylguanidine, and buffer containing (+)-pentazocine (2 μM (+)-pentazocine in 50 mM TRIS, pH 8.0) in a total volume of 200 μL for 180 min at room temperature. The incubation was terminated by rapid filtration through the presoaked filtermats using a cell harvester. After washing each well five times with 300 μL of water, the filtermats were dried at 95° C. Subsequently, the solid scintillator was placed on the filtermat and melted at 95° C. The bound radioactivity trapped on the filters was counted in the scintillation analyzer as described in the “General procedures” section. The non-specific binding was determined with 10 μM unlabeled ditolylguanidine. The K_{d}value of ditolylguanidine is 17.9 nM. The determination of the affinity to the σ₂ receptor was performed for the compounds according to formulas (1) to (28).

It was observed that the compounds according to formulas (1) to (3), (5), (7) to (14), (16), (17), (18), and (20) to (28), showed an affinity to the σ₂ receptor of 554±221 nM, 108±90 nM, 81.6±44.4 nM, 305 nM, 308±116 nM, 1.52 μM, 1.05 μM, 3.04 μM, 103 nM, 653 nM, 6.43 μM, 267±55 nM, 1.58 μM, 6.08±1.48 nM, 18.5±4.2 nM, 311 nM, 8.56±4.33 nM, 5.89±2.86 nM, 8.93±2.57 nM, 33.5±21.5 nM, 753 nM, 97.7 nM, 142 nM, and 103 nM, respectively, wherein the compounds according to formulas (4), (6), (15), and (19) showed inhibition of the σ₂ receptor only at concentrations above 1 μm.

This shows that the compounds according to formulas (1) to (3), (5), (7) to (14), (16), (17), (18), and (20) to (28), showed a good affinity to the σ₂ receptor, too.

### Example 37

### Treatment of Immune-Mediated Inflammatory Diseases

Glutamate-mediated excitotoxicity and neurodegeneration have been shown as pathophysiological hallmarks of multiple sclerosis and other autoimmune inflammatory CNS disorders. NMDA (N Methyl D Aspartate) receptors play a pivotal role in the mediation of neuronal glutamate excitotoxicity promoting an increased Ca2+ influx upon glutamate binding ultimately leading to cellular damage and apoptotic cell death. Current treatment approaches targeting glutamate excitotoxicity are unspecific and associated with severe adverse events due to the broad and important functions of NMDA receptors in the CNS. Hence, the present inventors investigated the therapeutic potential of a novel specific NR2B (NMDA receptor 2B) subunit antagonist.

The effects of the NR2B antagonist WMS14 10 (WMS) were investigated in MOG-EAE (myelin oligodendrocyte glycoprotein-induced experimental autoimmune encephalomyelitis), a murine model of multiple sclerosis, and in vitro for several immune cell subsets including isolated murine microglia. Flow cytometry, immunohistochemistry, ELISA, proliferation assays and RT-PCR were used as readout parameters.

Treatment with WMS significantly ameliorated the EAE disease course upon prophylactic and therapeutic administration. At disease maximum microglia from WMS treated mice showed decreased CD86 expression indicating reduced microglial activation. In agreement, activated microglia upregulated NR2B in vitro and in vivo. Under restimulation with MOG splenocytes from WMS treated mice demonstrated decreased secretion of TNFα, INFγ and IL-17.

In vitro, WMS showed no significant effects on the function of T cells and macrophages/monocytes. However, incubation with WMS reduced LPS (lipopolysaccharide)-mediated activation of microglia as assessed by CD86 and MHCII expression in flow cytometry.

In conclusion, the results provide evidence for a therapeutic potential of WMS14 10, a novel highly specific NR2B inhibitor, in the EAE model. Our results indicate that inhibition of NR2B in microglia cells displays a newly identified pathway in neuroinflammatory degeneration.

### Clinical Outcome of WMS14-10 Treatment of EAE Mice

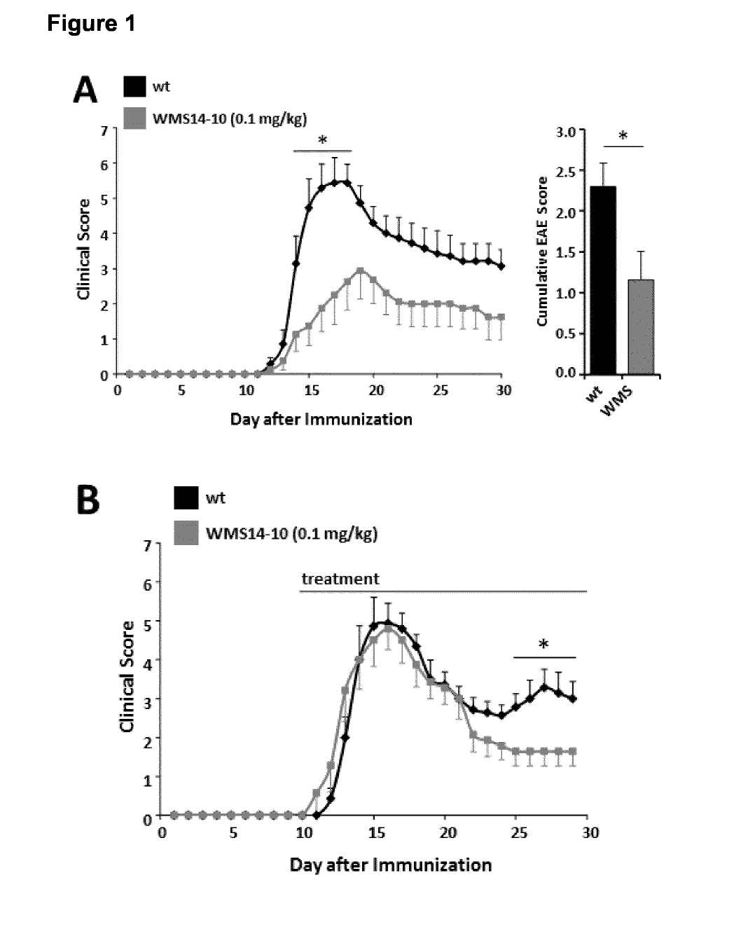

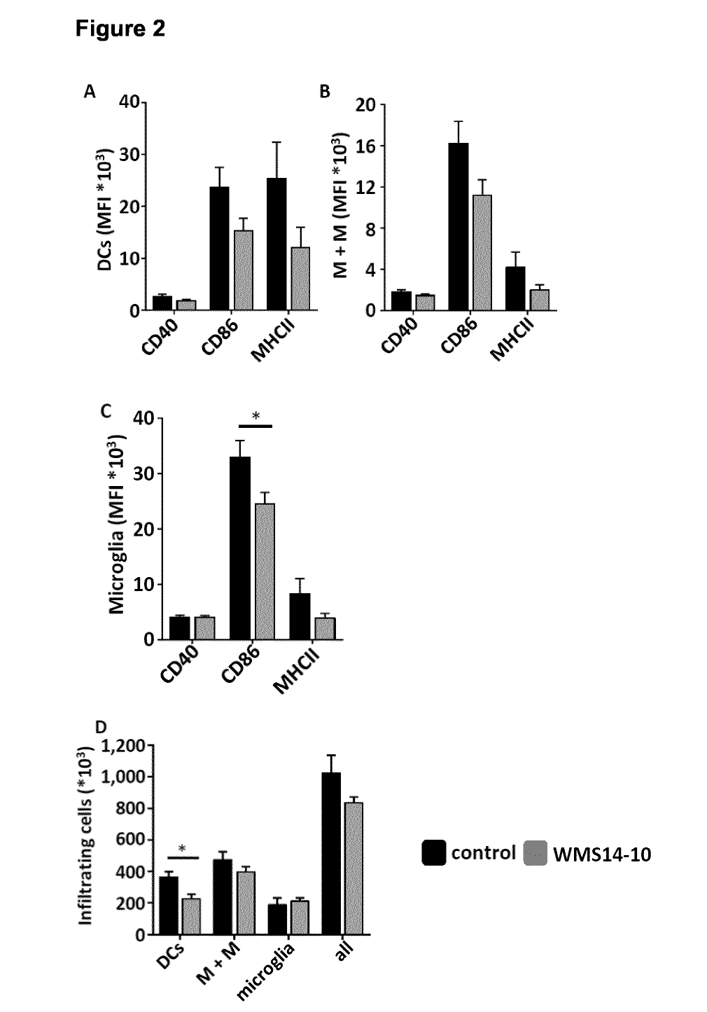

Experimental autoimmune encephalomyelitis was induced by immunization of female 10-12 week old C57BL/6 mice with 200 μg of a myelin oligodendrocyte glycoprotein (MOG₃₅₋₅₅) peptide and Freund's adjuvant emulsion. 400 ng pertussis toxin was injected at the day of immunization and two days later. Treatment with WMS14-10 was applied intraperitoneally either from the day of immunization (preventive treatment, FIG. 1A) or starting when animals showed clinical signs of EAE (therapeutic regimen, FIG. 1B). Disease progression was evaluated according to a clinical scoring system (0: no symptoms, 2: tail paralysis, 3: moderate hind limb weakness, 4: severe hind limb weakness, 5: complete hind limb paralysis, 6: paresis of the hind legs and forelegs). Administration of a low dose of WMS14-10 after immunization resulted in a markedly less severe course of disease. Even if treatment with WMS14-10 is begun after onset of symptoms, a positive effect is achieved (treatment).

### Immune Cell Activation

When mice showed maximal clinical signs of disease, mononuclear cells were isolated from the CNS and analysed by flow cytometry for different cell populations and expression of typical activation markers (CD40, CD86, MHCII). At disease maximum dendritic cells (DCs) (A) as well as monocytes/macrophages (M+M) (CD11b⁺ CD11c⁻) (B) from WMS treated mice brains show a trend of decreased activation markers in flow cytometry. Microglia (CD11b⁺CD45^{low}) from WMS treated mice expressing significantly less CD86 (C) indicate reduced microglia activation. Furthermore less DCs infiltrate the brain of treated mice (D).

### Splenocyte Activation and Cytokine Secretion

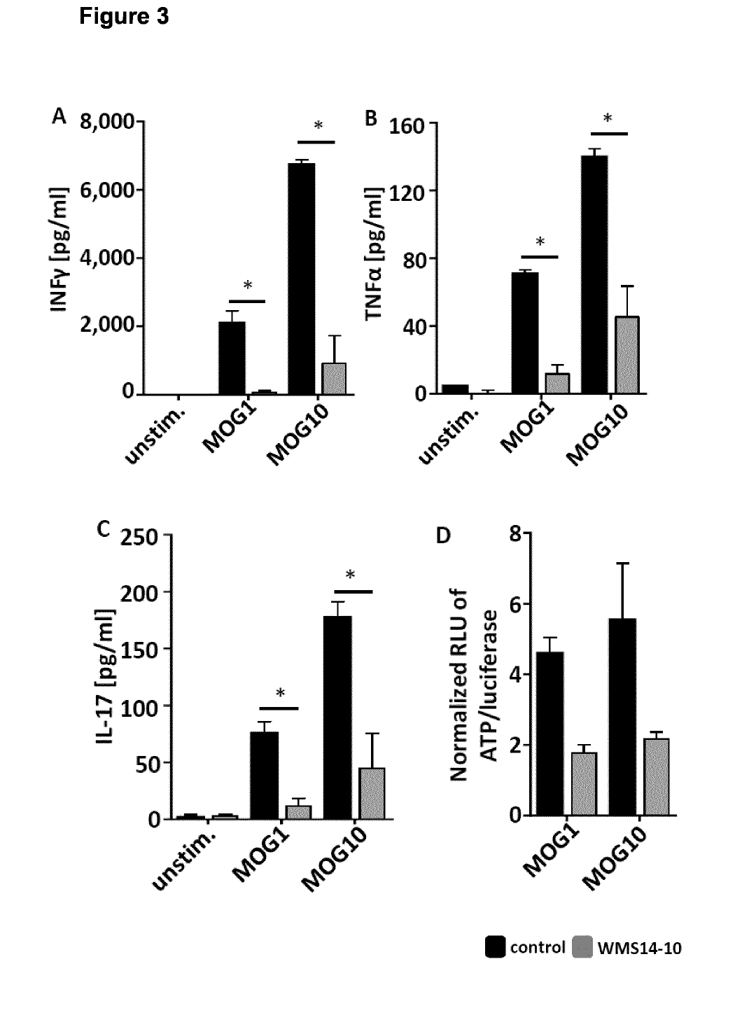

Splenocytes were isolated at the peak of disease (d15), restimulated with 1 μg/ml MOG peptide (MOG1) and 10 μg/ml MOG peptide (MOG10) and supernatants were analyzed for cytokines (TNFα, IFNγ and IL-17) by ELISA. Cytokine production was decreased in WMS-treated EAE mice compared to untreated EAE mice (FIG. 3 A-C). The proliferation rate, measured as relative light units (RLU) of ATP per luciferase, of splenocytes from treated mice also decreases after restimulation with MOG (D).

### NR2B Expression in the CNS

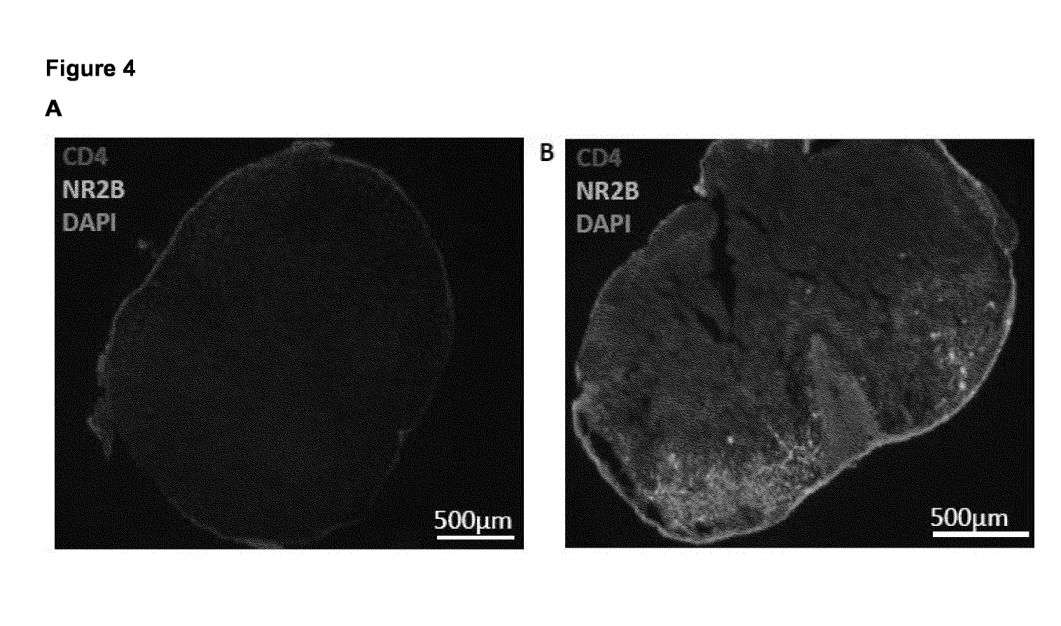

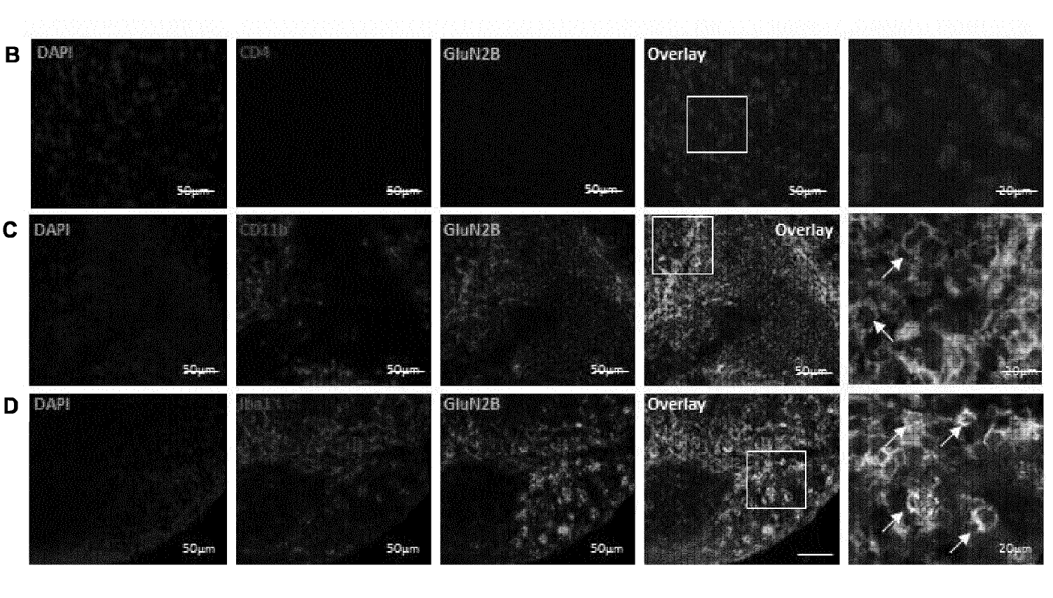

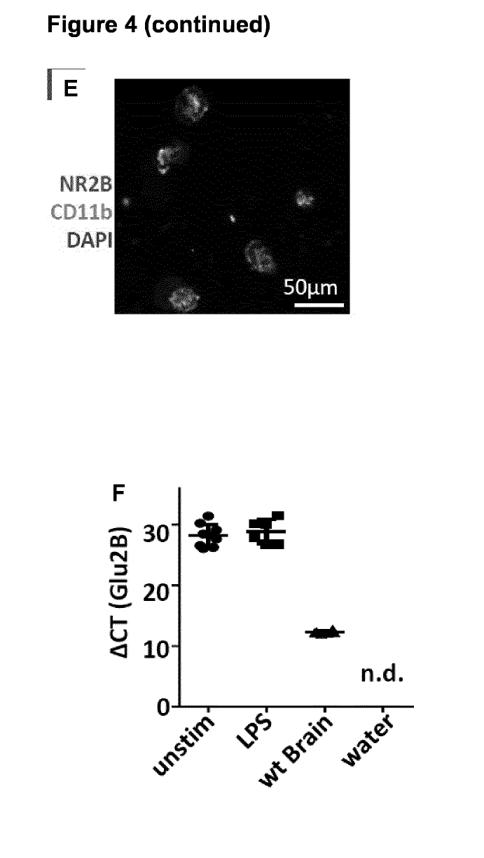

Spinal cords were isolated for immunohistochemical stainings for CD11b, Iba1 and NMDA receptor 2B (GluN2B) (FIG. 4). NR2B expression could be detected in spinal cords of healthy C57BL/6 mice (FIG. 4A, B). In contrast, at the peak of diseases (d15), NR2B expression (NR2B mouse anti mouse antibody; 1:400; StressMarq Biosciences Inc., Victoria, Canada) can be detected (FIG. 4 A, C, D; E) and correlates with the expression of CD11b—a marker for monocytes, macrophages and microglia (FIG. 4C, E)—and in particular for Iba1 indicating microglia activation (FIG. 4D). In agreement, either unstimulated or with 100 ng/ml 2100 LPS stimulated primary microglia show GluN2B mRNA expression (FIG. 4F).

### Reduced Microglia Activation in WMS14-10 Treated Mice.

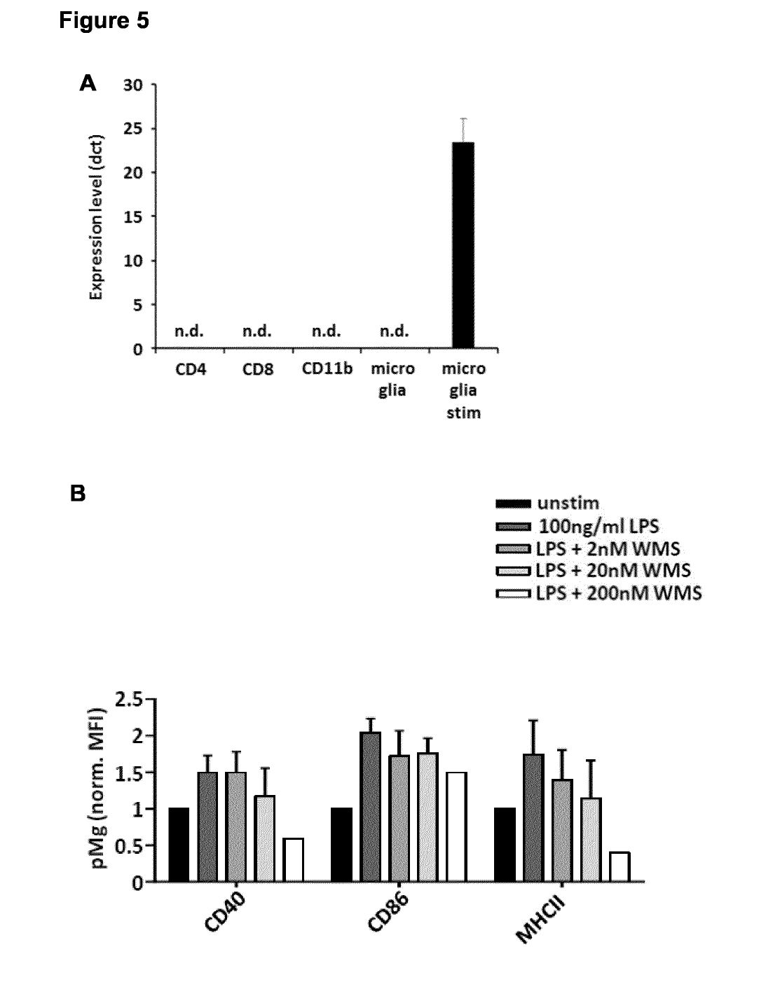

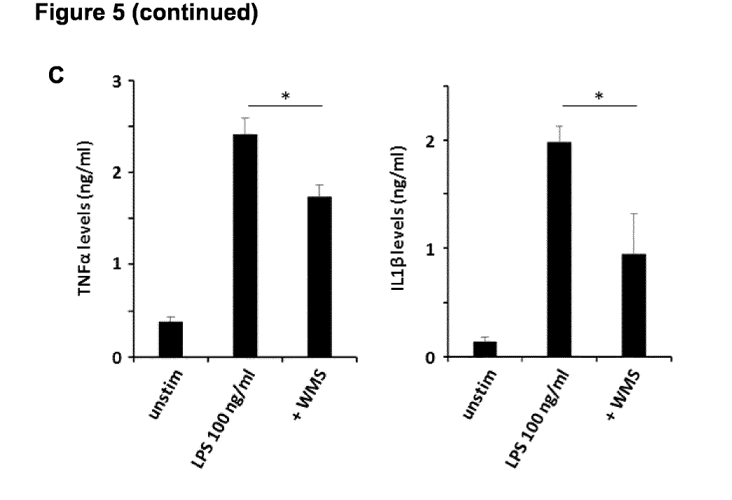

CD4⁺, CD8⁺and CD11b⁺ cells were isolated from the spleen of naïve CD57BL/6 mice and compared with unstimulated or stimulated primary murine microglia cultures for NR2B expression levels by RT-PCR (FIG. 5). Upon stimulation with 100 ng/ml LPS, primary murine microglia upregulate activation markers CD40, CD86 and MHCII. Application of 2, 20 and 200 nM WMS inhibits upregulation of activation markers (FIG. 5B) and reduces secreted levels of TNFα and IL1β as assessed by ELISA (FIG. 5C).

### Effect of Treatment

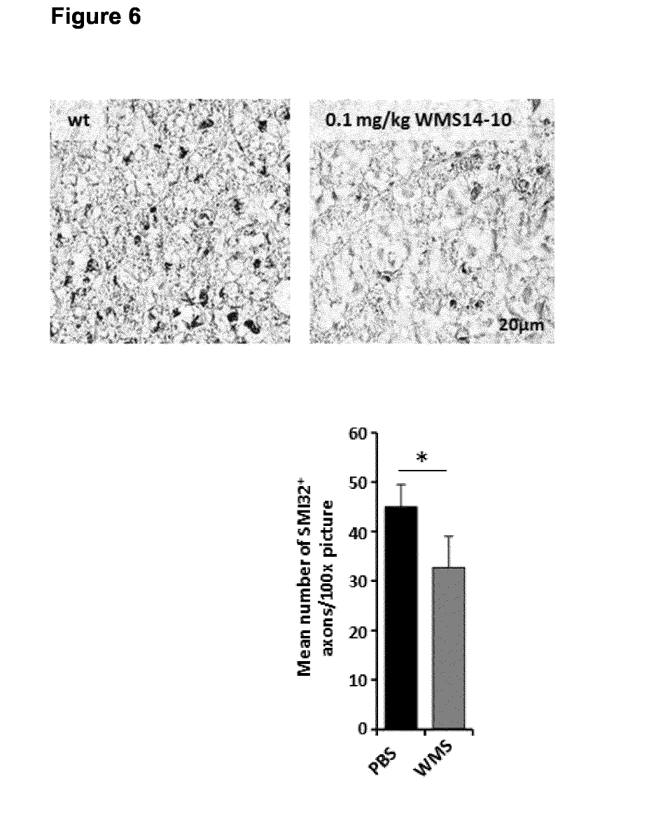

30 days after immunization, spinal cords were isolated and analyzed for SMI32 positive axons by immunocytochemistry. WMS14-10-mediated inhibition of NR2B resulted in reduced loss of neurons on d30 (FIG. 6).

1. Compounds according to the general formula (I) 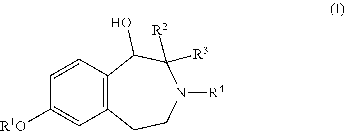
for use in a method of therapeutic and/or prophylactic treatment of immune-mediated inflammatory diseases
wherein:
R¹ is selected from the group comprising hydrogen; linear or branched C₁-C₈-alkyl;
C₂-C₈-alkenyl; C₃-C₈-cycloalkyl; C₈-C₁₀-aryl; C₄-C₁₀-cycloalkylalkyl wherein the cycloalkyl group has 3 to 6 carbon atoms and the alkyl group has 1 to 4 carbon atoms; and/or C₇-C₁₄-arylalkyl wherein the aryl group has 6 to 10 carbon atoms and the alkyl group has 1 to 4 carbon atoms;
R² is selected from the group comprising hydrogen; linear or branched C₁-C₈-alkyl; C₂-C₈-alkenyl; C₃-C₈-cycloalkyl; C₈-C₁₀-aryl; C₄-C₁₀-cycloalkylalkyl wherein the cycloalkyl group has 3 to 6 carbon atoms and the alkyl group has 1 to 4 carbon atoms; C₇-C₁₄-arylalkyl wherein the aryl group has 6 to 10 carbon atoms and the alkyl group has 1 to 4 carbon atoms;
R³ is selected from the group comprising hydrogen; linear or branched C₁-C₈-alkyl; C₂-C₈-alkenyl; C₃-C₈-cycloalkyl; C₈-C₁₀-aryl; C₄-C₁₀-cycloalkylalkyl wherein the cycloalkyl group has 3 to 6 carbon atoms and the alkyl group has 1 to 4 carbon atoms; C₇-C₁₄-arylalkyl wherein the aryl group has 6 to 10 carbon atoms and the alkyl group has 1 to 4 carbon atoms; linear or branched alkyl groups of the type —CₙH₂ₙ—U-D wherein n is 1 , 2, 3 or 4, U is selected from the group comprising O, CO, COO, CONH, S, guanidine and/or NH and D is selected from the group comprising H and/or C₁-C₃-alkyl; —CH₂-C₆H₄—X wherein X is selected from the group comprising OH, SH, C₁-C₃-alkyl and/or NH₂; —CH₂-imidazole; —CH₂-indole; —CH₂-(furanyl-3-yl); —CH₂— (pyridyl-3-yl) and/or —CH₂-(imidazolyl-3-yl); or
R² and R³ together with the carbon atom to which they are attached form a 5- to 7-membered non aromatic carbocycle or heterocycle comprising from 1 to 3 hetero atoms selected from the group comprising O, N and/or S;
R⁴ is selected from the group comprising hydrogen; C₁-C₁₀-alkyl; —W and/or —Y—Z;
Y is selected from the group comprising C₁-C₆-alkyl; C₂-C₆-alkenyl; C₂-C₆-alkynyl, C₃-C₆-cycloalkyl; C₄-C₁₀-cycloalkylalkyl wherein the cycloalkyl group has 3 to 6 carbon atoms and the alkyl group has 1 to 4 carbon atoms; C₆-C₁₀-aryl; C₇-C₁₄-arylalkyl wherein the aryl group has 6 to 10 carbon atoms and the alkyl group has 1 to 4 carbon atoms;
C₁-C₆-alkyl comprising at least one moiety independently selected from the group comprising oxygen, sulphur, SO, SO₂, CO, NH, N(C₁-C₃-alkyl) and/or T;
a 3- to 6-membered aromatic or non aromatic carbocycle or heterocycle containing at least one of O, N or S as heteroatoms; and/or a structural element comprising a 3- to 6-membered aromatic or non aromatic carbocycle or heterocycle comprising from 1 to 3 heteroatoms selected from the group comprising O, N and/or S and a group selected from the group comprising oxygen, sulphur, SO, SO₂, CO, NH, N(C₁-C₃-alkyl) and/or C₁-C₆-alkyl comprising at least one moiety independently selected from the group comprising oxygen, sulphur, SO, SO₂, CO, NH and/or N(C₁-C₃-alkyl);
T is selected from the group comprising 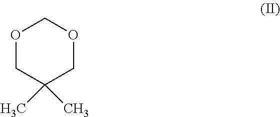
W is selected from the group comprising 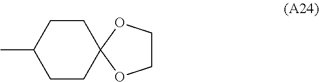
Z is selected from the group comprising mono-, bi- or tricyclic aromatic or non aromatic carbocycles or heterocycles comprising from 1 to 3 heteroatoms selected from the group comprising O, N and/or S, wherein the carbocycle or heterocycle optionally is substituted by at least one group selected from the group comprising halogen, cyano, OH, CF₃, C₁-C₄-alkyloxy and/or C₁C₆-alkyl; or
R³ and R⁴ together with the ring atoms to which they are attached form a 5- to 7-membered non aromatic heterocycle comprising from 1 to 3 heteroatoms selected from the group comprising O, N and/or S;
and/or racemates, enantiomers, diastereomers, solvates, hydrates, and pharmaceutically acceptable salts and/or esters thereof.

2. Compounds for the use according to claim 1, characterized in that
R² is hydrogen and R³ is a side chain of an amino acid selected from the group comprising hydrogen; linear or branched C₁-C₄-alkyl; linear or branched alkyl groups of the type —CₙH₂ₙ—U-D wherein n is 1, 2, 3 or 4, U is selected from the group comprising O, CO, COO, CONH, S, guanidine and/or NH, and D is selected from the group comprising H and/or methyl; —CH₂—C₆H₄—OH; —CH₂-imidazole and/or —CH₂-indole; or
R³ and R⁴ together with the ring atoms to which they are attached form a 5-membered non aromatic heterocycle having one N atom;

3. Compounds for the use according to any one of claim 1 or 2, characterized in that
R⁴ is selected from the group comprising the structural elements as given as follows:

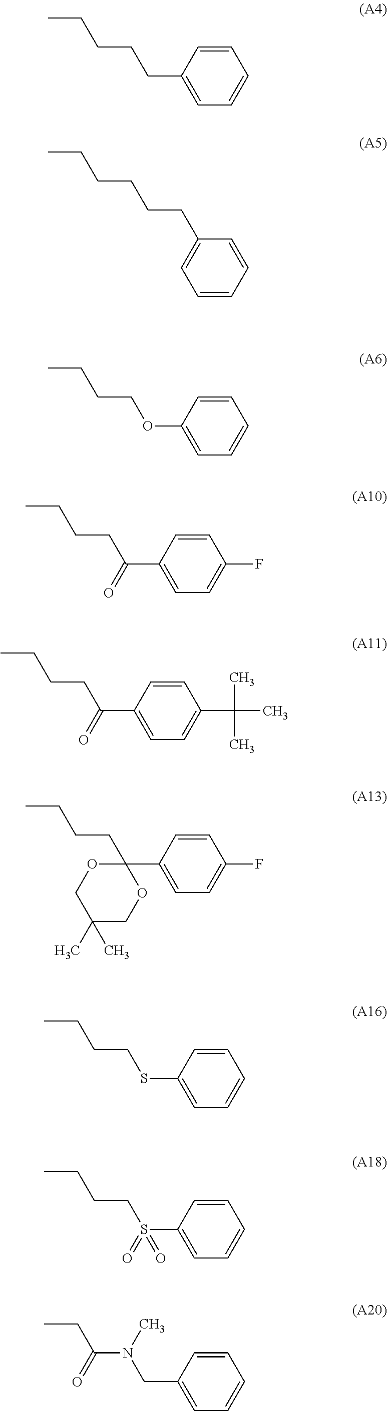

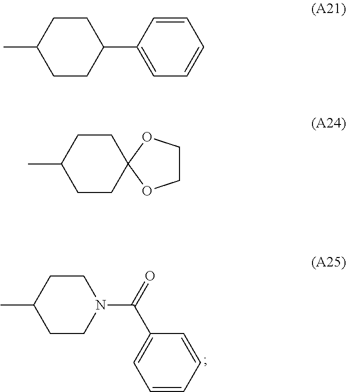

4. Compounds for the use according to any one of the preceding claims, characterized in that the compound is a compounds according to general formula (III) as given as follows 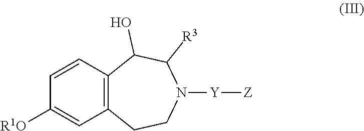
wherein:
R¹ is selected from the group comprising hydrogen; linear or branched C₁-C₆-alkyl and/or benzyl;
R³ is a side chain of an amino acid selected from the group comprising hydrogen; linear or branched C₁-C₄-alkyl; linear or branched alkyl groups of the type —CₙH₂ₙ—U-D wherein n is 1, 2, 3 or 4, U is selected from the group comprising O, CO, COO, CONH, S, guanidine and/or NH, and D is selected from the group comprising H and/or methyl; —CH₂—C₆H₄13 OH; —CH₂-imidazole and/or —CH₂-indole;
Y is selected from the group comprising-(CH₂)ₘ— wherein m represents 3, 4 or 5; C₃-C₅-alkenyl; C₅-C₆-cycloalkyl; C₃-C₅-alkyl comprising at least one moiety independently selected from the group comprising oxygen, sulphur, SO, SO₂, CO, NH and/or N(C₁C₃-alkyl);
a 5- to 6-membered non aromatic carbocycle or heterocycle comprising from 1 to 3 heteroatoms selected from the group comprising O, N and/or S;
and/or a structural element comprising a 5- to 6-membered non aromatic carbocycle and a group selected from the group comprising oxygen, sulphur, SO, SO₂, CO, NH, N(C₁-C₃-alkyl) and/or C₁-C₃-alkyl comprising at least one moiety independently selected from the group comprising oxygen, sulphur, SO, SO₂, CO, NH and/or N(C₁-C₃-alkyl);
Z is selected from the group comprising mono-, bi- or tricyclic aromatic carbocycles or heterocycles comprising from 1 to 3 heteroatoms selected from the group comprising O, N and/or S, wherein the carbocycle or heterocycle optionally is substituted by at least one group selected from the group comprising halogen, cyano, OH, CF₃, Ci-C₄-alkyloxy and/or C₁-C₆-alkyl;

5. Compounds for the use according to any one of the preceding claims, characterized in that the compound is selected from the group comprising compounds according to the formulas as given as follows:

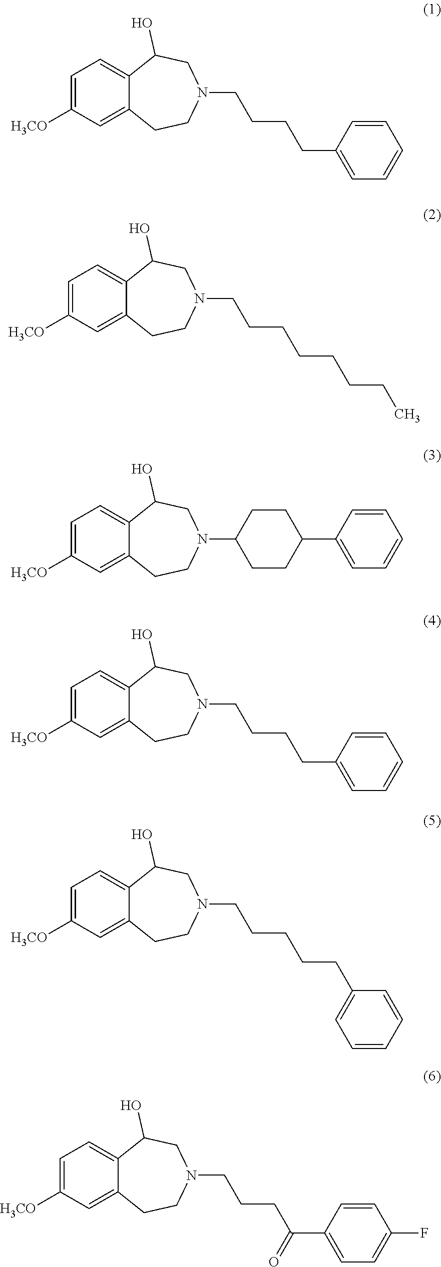 [SMILES: COc1ccc2c(c1)CCN(CCCCc1ccccc1)CC2O ; CCCCCCCCN1CCc2cc(OC)ccc2C(O)C1 ; COc1ccc2c(c1)CCN(C1CCC(c3ccccc3)CC1)CC2O ; COc1ccc2c(c1)CCN(CCCCc1ccccc1)CC2O ; COc1ccc2c(c1)CCN(CCCCCc1ccccc1)CC2O ; COc1ccc2c(c1)CCN(CCCC(=O)c1ccc(F)cc1)CC2O]

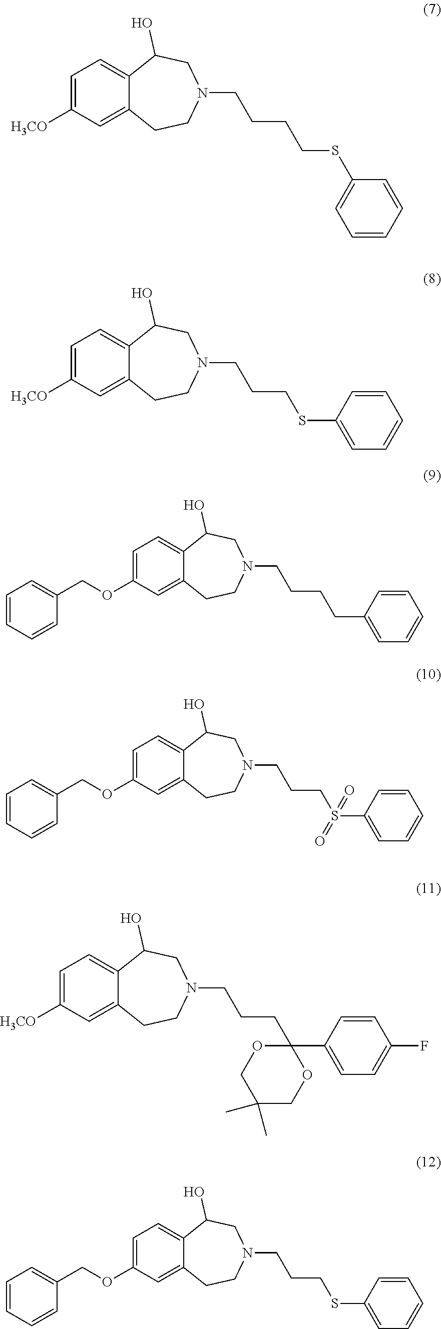 [SMILES: COc1ccc2c(c1)CCN(CCCCSc1ccccc1)CC2O ; COc1ccc2c(c1)CCN(CCCSc1ccccc1)CC2O ; OC1CN(CCCCc2ccccc2)CCc2cc(OCc3ccccc3)ccc21 ; O=S(=O)(CCCN1CCc2cc(OCc3ccccc3)ccc2C(O)C1)c1ccccc1 ; COc1ccc2c(c1)CCN(CCCC1(c3ccc(F)cc3)OCC(C)(C)CO1)CC2O ; OC1CN(CCCSc2ccccc2)CCc2cc(OCc3ccccc3)ccc21]

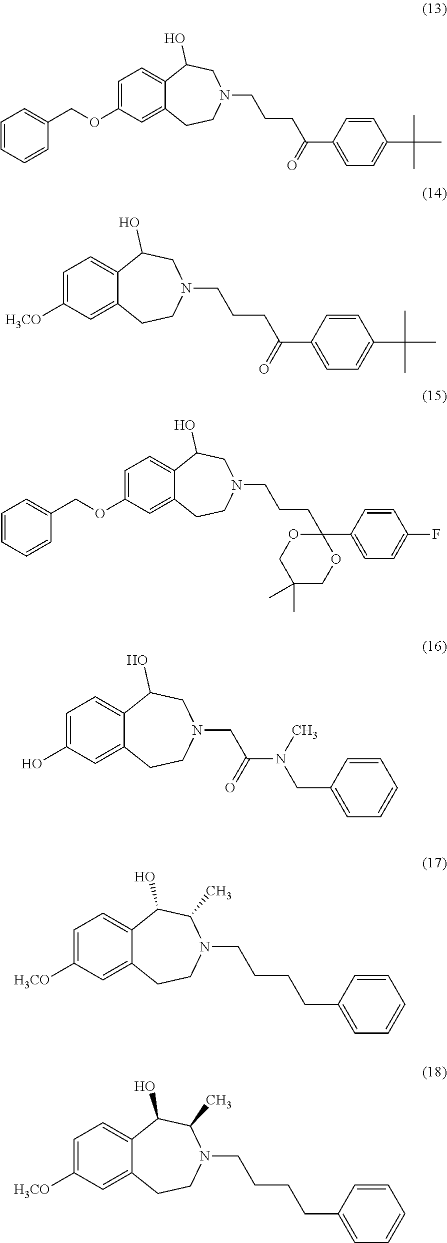 [SMILES: CC(C)(C)c1ccc(C(=O)CCCN2CCc3cc(OCc4ccccc4)ccc3C(O)C2)cc1 ; COc1ccc2c(c1)CCN(CCCC(=O)c1ccc(C(C)(C)C)cc1)CC2O ; CC1(C)COC(CCCN2CCc3cc(OCc4ccccc4)ccc3C(O)C2)(c2ccc(F)cc2)OC1 ; CN(Cc1ccccc1)C(=O)CN1CCc2cc(O)ccc2C(O)C1 ; COc1ccc2c(c1)CCN(CCCCc1ccccc1)[C@@H](C)[C@H]2O ; COc1ccc2c(c1)CCN(CCCCc1ccccc1)[C@H](C)[C@@H]2O]

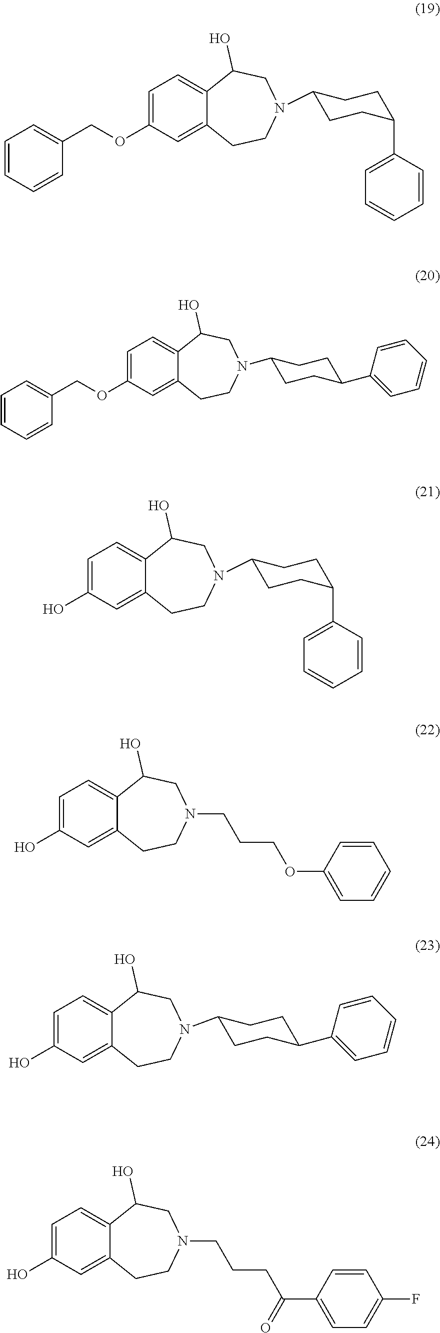 [SMILES: OC1CN(C2CCC(c3ccccc3)CC2)CCc2cc(OCc3ccccc3)ccc21 ; OC1CN(C2CCC(c3ccccc3)CC2)CCc2cc(OCc3ccccc3)ccc21 ; Oc1ccc2c(c1)CCN(C1CCC(c3ccccc3)CC1)CC2O ; Oc1ccc2c(c1)CCN(CCCOc1ccccc1)CC2O ; Oc1ccc2c(c1)CCN(C1CCC(c3ccccc3)CC1)CC2O ; O=C(CCCN1CCc2cc(O)ccc2C(O)C1)c1ccc(F)cc1]

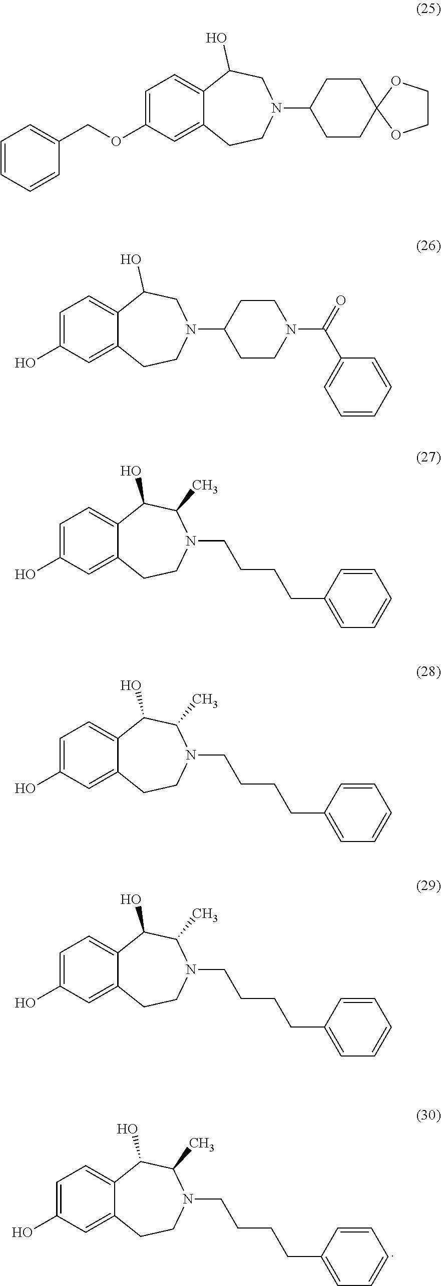 [SMILES: OC1CN(C2CCC3(CC2)OCCO3)CCc2cc(OCc3ccccc3)ccc21 ; O=C(c1ccccc1)N1CCC(N2CCc3cc(O)ccc3C(O)C2)CC1 ; C[C@@H]1[C@H](O)c2ccc(O)cc2CCN1CCCCc1ccccc1 ; C[C@H]1[C@@H](O)c2ccc(O)cc2CCN1CCCCc1ccccc1 ; C[C@H]1[C@H](O)c2ccc(O)cc2CCN1CCCCc1ccccc1 ; C[C@@H]1[C@@H](O)c2ccc(O)cc2CCN1CCCCc1ccccc1]

6. Compounds for the use according to any one of the preceding claims, wherein the immune-mediated inflammatory diseases are characterized by one or more of the following features:
(v) Overexpession of proinflammatory cytokines, preferably IL-1, IL-6, and/or IFNγ, IL-17, TNF-α; and/or overexpression of autoantibodies
(vi) Th1/Th2 cytokine disbalance; and/or Th17 disbalance and/or changes in Treg function
(vii) Autoimmune responses; and/or
(viii) Amelioration of disease symptoms by immunosuppressive therapies

7. Compounds for the use according to any one of the preceding claims, wherein the immune-inflammatory diseases are selected from multiple sclerosis, rheumatoid arthritis, Crohn's disease, psoriasis, psoriatic arthritis, inflammatory bowel disease (IBD), ulcerative colitis (UC), systemic lupus erythematosus (SLE), Sjogren syndrome, ANCA-induced vasculitis, ankylosing spondylitis, anti-phospholipid syndrome, myasthenia gravis, Addison's disease, alopecia areata, ankylosing spondylitis, antiphospholipid antibody syndrome, antiphospholipid syndrome (primary or secondary), asthma, autoimmune gastritis, autoimmune hemolytic anemia, autoimmune hepatitis, autoimmune inner ear disease, autoimmune lymphoproliferative disease, autoimmune thrombocytopenic purpura, Balo disease, Behcet's disease, bullous pemphigoid, cardiomyopathy, celiac disease, Chagas disease, chronic inflammatory demyelinating polyneuropathy, cicatrical pemphigoid, cold agglutinin disease, degos disease, dermatitis hepatiformis, essential mixed cryoglobulinemia, Goodpasture's syndrome, Graves' disease, Guillain-Barre syndrome, Hashimoto's thyroiditis, idiopathic pulmonary fibrosis, idiopathic thrombocytopenia purpura, IgA nephropathy, juvenile arthritis, lichen planus, Meniere disease, mixed connective tissue disease, morephea, neuromyotonia, pemphigus vulgaris, pernicious anemia, polyarteritis nodosa, polychondritis, polymyalgia rheumatica, primary agammaglobulinemia, primary biliary cirrhosis, Raynaud disease (Raynaud phenomenon), Reiter's syndrome, relapsing polychondritis, rheumatic fever, Sjogren's syndrome, stiff-person syndrome (Moersch-Woltmann syndrome), Takayasu's arteritis, temporal arteritis (giant cell arteritis), uveitis, vasculitis, vitiligo, Wegener's granulomatosis and/or neuromyelitis optica, isolated CNS-vasculitis.

8. Compounds for the use according to any one of the preceding claims, wherein the subject to be treated has been pre-diagnosed with an immune-inflammatory disease according to claim 7.

9. Compounds for the use according to any of the preceding claims, wherein treatment results in reduced activation of immune cells.

10. Compounds for the use according to according to claim 9, wherein immune cells are selected from macrophages, monocytes, microglia and/or dendritic cells.

11. Compounds for the use of any one of the preceding claims, wherein treatment results in levels of
(i) surface CD40: and/or
(ii) surface CD86; and/or
(iii) surface MHCII and/or
(iv) surface CD80
on immune cells.

12. Compounds for the use according to any one of the preceding claims, wherein treatment results in levels of
(i) TNFalpha; and/or
(ii) IFNgamma; and/or
(iii) IL18; and/or
(iv) IL6
from immune cells.

13. Compounds for the use of any one of the foregoing claims, wherein the compounds act as NR2B-selective NMDA receptor antagonists.

14. Pharmaceutical composition comprising as an active ingredient a compound according to any one of the claims 1 to 13 and/or racemates, enantiomers, diastereomers, solvates, hydrates, pharmaceutically acceptable salts and/or esters thereof for use in a method of treatment of immune-mediated inflammatory diseases.

15. The pharmaceutical composition according to claim 14, further comprising a pharmaceutically acceptable excipient.

16. The pharmaceutical composition according to any one of claim 14 or 15, further comprising corticosteroids, including prednisone and methylprednisolone, beta interferons, glatiramer acetate, dimethyl fumarate, fingolimod, teriflunomide, natalizumab, mitoxantrone, infliximab, etanercept, adalimumab, rituximab, abatacept, anakinra, alefacept and/or efalizumab.

17. Kit comprising a compound according to any one of claims 1 to 13 and one or more of corticosteroids, including prednisone and methylprednisolone, beta interferons, glatiramer acetate, dimethyl fumarate, fingolimod, teriflunomide, natalizumab, mitoxantrone, infliximab, etanercept, adalimumab, rituximab, abatacept, anakinra, alefacept and/or efalizumab.

18. Use of a compound according to any one of claims 1 to 13 for prophylactic and/or therapeutic treatment of immune-mediated inflammatory diseases.

19. Use of a compound according to any one of claims 1 to 13 for manufacturing a medicament for therapeutic and/or prophylactic treatment of immune-mediated inflammatory diseases.

20. A method of treating immune-mediated inflammatory diseases comprising administering a compound according to any one of claims 1 to 13 or a pharmaceutical composition according to any one of claims 14 to 16 to a subject.

21. NR2B-selective NMDA receptor antagonists for use in a method of treatment of multiple sclerosis.
